(19) United States
(12) Patent Application Publication
(10) Pub. No.: US 2019/0060371 A1
(43) Pub. Date: Feb. 28, 2019

(54) METHODS FOR CONTROLLED PROLIFERATION OF VESTIBULAR STEM CELLS / GENERATING INNER EAR HAIR CELLS USING WNT AND TGFBETA-INHIBITION

(71) Applicant: Frequency Therapeutics, Inc., Woburn, MA (US)

(72) Inventor: Will MCLEAN, North Haven, CT (US)

(21) Appl. No.: 16/080,820

(22) PCT Filed: Mar. 2, 2017
(86) PCT No.: PCT/US2017/020437

§ 371 Date: Aug. 29, 2018

Related U.S. Application Data
(60) Provisional application No. 62/302,799, filed on Mar. 2, 2016.
(60) Provisional application No. 62/303,035, filed on Mar. 3, 2016.

(51) Int. Cl. A61K 35/30; A61K 9/00; A61L 27/52; A61P 27/16; A61L 27/56

(52) U.S. Cl. CPC A61K 35/30; A61K 9/0046; A61L 27/56; A61P 27/16; A61L 27/52

(57) ABSTRACT

Provided are compositions and methods for using Wnt agonist, GSK3-alpha inhibitor, or GSK3-beta inhibitors in combination with TGF-beta inhibitors to induce the self-renewal of stem/progenitor supporting cells, including inducing the stem/progenitor cells to proliferate while maintaining in the daughter cells the capacity to differentiate into hair cells.

### CROSS-REFERENCE TO RELATED APPLICATIONS

This application claims priority under 35 U.S.C. § 119(e) to U.S. Application No. 62/302,799, filed Mar. 2, 2016; and U.S. Application No. 62/303,035, filed Mar. 3, 2016, each of which is incorporated by reference in its entirety.

### BACKGROUND

### Technical Field

The present disclosure relates to compositions and methods for inducing the self-renewal of stem/progenitor supporting cells, for example, vestibular cells, including inducing the stem/progenitor cells to proliferate while maintaining in the daughter cells the capacity to differentiate into tissue cells.

### Description of the Related Art

Stem cells exhibit an extraordinary ability to generate multiple cell types in the body. Besides embryonic stem cells, tissue specific stem cells serve a critical role during development as well as in homeostasis and injury repair in the adult. Stem cells renew themselves through proliferation as well as generate tissue specific cell types through differentiation. The characteristics of different stem cells varies from tissue to tissue, and are determined by their intrinsic genetic and epigenetic status. However, the balance between self-renewal and differentiation of different stem cells are all stringently controlled. Uncontrolled self-renewal may lead to overgrowth of stem cells and possibly tumor formation, while uncontrolled differentiation may exhaust the stem cell pool, leading to an impaired ability to sustain tissue homeostasis. Thus, stem cells continuously sense their environment and appropriately respond with proliferation, differentiation, or apoptosis. It would be desirable to drive regeneration by controlling the timing and extent of stem cell proliferation and differentiation. Controlling the proliferation with small molecules that are cleared over time would allow for control of the timing and extent of stem cell proliferation and differentiation. Remarkably, tissue stem cells from different tissues share a limited number of signaling pathways for the regulation of their self-renewal and differentiation, albeit in a very context dependent manner. One of these pathways is the Wnt pathway.

Wnt stimulation has been known to drive proliferation of stem cells and also differentiate stem cells. Both roles have been seen in the cochlea (Shi et al, 2104) depending on age. Further, Wnt activation has been shown to drive proliferation in the utricle (Lu et al 2008).

Several stem cell genes are known to be involved in Wnt stimulation. Some of these include Sox9, Sox2, Lgr5, Frizzled, Lgr4, Pax2, Pax6, Pax8, and Bmi1.

Sox9 and Sox2 have been shown to have differential and overlapping expression in the developing vestibular organs. Sox9 marks supporting cells while Sox2 marks supporting cells and Type II hair cells (Mak et al. 2009).

Lgr5 is expressed across a diverse range of tissues and has been identified as a biomarker of adult stem cells in the gut epithelia (Barker et al. 2007), kidney, hair follicle, and stomach (Barker et al, 2010; Haegebarth & Clevers, 2009). For example, it was first published in 2011, that mammalian cochlear hair cells are derived from supporting cells (Chai et al, 2011, Shi et al. 2012). Lgr5 is a known component of the Wnt/beta-catenin pathway, which has been shown to play major roles in differentiation, proliferation, and inducing stem cell characteristics (Barker et al. 2007).

15% of Americans suffer from impaired hearing and 35% from vestibular and balance problems that are correlated with a risk of falling and severely reduced quality of life (Agrawal et al., 2009). Sensory hair cell loss from trauma, aging, ototoxic drugs, or congenital abnormalities is a major factor in hearing and balance dysfunction (reviewed in Wong and Ryan, 2015). Once lost, auditory hair cells do not regenerate in mammals. A low level of regeneration of vestibular hair cells has been observed (Burns et al., 2012) but does not fully compensate for cell loss correlated with aging (Rauch et al., 2001; Burns et al., 2012). Regeneration of damaged hair cells would provide an avenue for the treatment conditions that currently has no therapies other than prosthetic devices. Although hair cells do not regenerate in the mammalian cochlea, new hair cells in lower vertebrates are generated from epithelial cells, called supporting cells, that surround hair cells.

Prior work has focused on transdifferentiation of supporting cells into hair cells through activation or forced expression of genes that lead to hair cell formation, with a particular focus on mechanisms to enhance expression of Atoh1 (Bermingham et al., 1999; Zheng and Gao, 2000; Izumikawa et al., 2005; Mizutari et al., 2013). Interestingly, cells transduced with Atoh1 vectors have been shown to acquire “primordial” phenotypes (Kawamoto et al., 2003; Huang et al., 2009; Yang et al., 2012, 2013) that behave similarly to hair cells in lower vertebrates, and lack complete development into bona fide mammalian hair cells. As mentioned, upregulating Atoh1 via gene insertion has been shown to create non-cochlear cell types that behave in a manner that is not found within the native cochlea or vestibular end organs. In addition, these methods increase hair cell numbers but decrease supporting cell numbers. Since supporting cells are known to have specialized roles (Ramirez-Camancho 2006, Dale and Jagger 2010), loss of these cells could create problems in proper inner ear function.

Thus, there remains a long felt need to protect hair cells before injury, preserve/promote the function of existing cells after injury, and regenerate vestibular supporting cells or hair cells after injury. As disclosed below, in certain embodiments, the present disclosure provides methods for preventing and treating vestibular dysfunctions.

### BRIEF DESCRIPTION OF THE DRAWINGS

FIG. 1 shows transmitted light images of colonies in three-dimensional (3D) culture with various culture media conditions, as indicated (GF: Growth factors EGF, IGF, and FGF; C: CHIR99021; 6: 616452; V:VPA; N:Noggin).

FIG. 2 shows that (i) Wnt agonism (C) promoted supporting cell/stem cell growth, (ii) Wnt agonism (C)+TGF-beta inhibition (6) improved stem cell growth, and (iii) HDAC inhibition by valproic acid (VPA) inhibited cell growth.

FIG. 3 shows confocal images of clonal supporting cell colonies with the characteristic actin lattices indicative of supporting cells (red), and the stem cell/supporting cell marker Sox9 (green). Colonies grown in GFC6 appear larger than GFC colonies.

FIG. 4 shows confocal images in which clonal colonies were differentiated into high purity populations of hair cells using the gamma secretase inhibitor LY411575. Indicative of hair cells, the colonies express Myosin VIIA (green) and contain actin hair bundles (red).

FIG. 5 shows that in a background of GF, (i) Wnt agonism (CHIR99021) promoted supporting cell/stem cell growth, and (ii) Wnt agonism (CHIR99021)+TGF-beta inhibition using two alternative TGF-beta inhibitors (SB-431542 & A-83-01) generated larger colonies of supporting cells/stem cells compared to Wnt agonism alone.

### BRIEF SUMMARY

In one aspect the present disclosure provides a method for controlled proliferation of stem cells by promoting sternness to give proliferation and creation of daughter cells that differentiate comprising administering to a cell population an effective amount of (i) a Wnt agonist, GSK3-alpha inhibitor, or GSK3-beta inhibitor, and (ii) a TGF-β inhibitor.

Among the various aspects of the present disclosure, therefore, may be noted a method for activating the Wnt pathway in a cell population to increase the capacity of the population for self-renewal, i.e., the capacity for repeated generation of daughter cells with equivalent proliferation and ‘cell fate specification’ potential, and differentiation, i.e., the capacity for generation of daughter cells specified for differentiation. In one embodiment, the cell population is a vestibular supporting cell population. In some embodiments, the Wnt pathway is preferably activated upstream of the c-myc gene in members of the population and without any genetic modification of the population. In some such embodiments, the Wnt pathway is preferably activated by small molecules that transiently induce such activity. In certain embodiments, the Wnt pathway is activated by a Wnt agonist, a GSK3-alpha inhibitor, or a GSK3-beta inhibitor disclosed herein. Additionally, the supporting cell population preferably includes supporting cells that are endogenous to the Vestibular Organs.

A further aspect of the present disclosure is, in some embodiments, a method for inducing the self-renewal of stem/progenitor supporting cells comprised by a vestibular cell population. That is, the stem/progenitor supporting cells are induced to proliferate (i.e., divide and form daughter cells) while maintaining, in the daughter cells, the capacity to differentiate into hair cells. In contrast, if the stem/progenitor supporting cells were merely induced to proliferate (without maintaining multi-potency), the daughter cells would lack the capacity to differentiate into hair cells. Further, merely enforcing differentiation of a pre-existing stem/progenitor cell population has the potential to exhaust the stem cell pool. Accordingly, the present disclosure provides a method in which pre-existing vestibular supporting cells are induced to proliferate prior to differentiation and the expanded population is then permitted (or even induced in some embodiments) to differentiate into hair cells. In some embodiments, the Wnt pathway is preferably activated upstream of the c-myc gene in members of the population and without any genetic modification of the population. In some such embodiments, proliferation is preferably activated by small molecules that transiently induce such activity. In certain embodiments, the Wnt pathway is activated by a Wnt agonist, a GSK3-alpha inhibitor, or a GSK3-beta inhibitor disclosed herein. Additionally, in certain embodiments the supporting cell population preferably includes cells that are supporting cells and endogenous to the Vestibular Organs.

In an embodiment the Wnt pathway is activated with a Wnt agonist, GSK3-alpha inhibitor, or GSK3-beta inhibitor (e.g., any one of the Wnt agonist, GSK3-alpha inhibitor, or GSK3-beta inhibitors disclosed in Table 1, Table 6, and Table 2, respectively). In some embodiments, the Wnt agonist, GSK3-alpha inhibitor, or GSK3-beta inhibitor is CHIR99021, LY2090314, AZD1080, or GSK3 inhibitor XXII.

In various embodiments the methods of the present disclosure comprise both activation of the Wnt pathway with a Wnt agonist, GSK3-alpha inhibitor, or GSK3-beta inhibitor and inhibition of the TGF-beta pathway. In one such embodiment, the TGF-beta pathway is inhibited with a TGF-beta inhibitor. In some embodiments, the TGF-beta inhibitor is a TGF-beta type I receptor inhibitor, TGF-beta R1 kinase inhibitor, and/or an inhibitor of any one or more of ALK2/4/5/7. In some embodiments, the TGF-beta inhibitor is any one of the inhibitors listed in Table 5. In one embodiment, the TGF-beta inhibitor is selected from 616452 (Repsox), Galunisertib (LY2157299), EW-719, IN-1130, EW-7203, EW-7195, SM16, R 268712, GW788388, SB-431542, and PF-03671148. In some embodiments, the methods of the present disclosure further comprises inhibiting a BMP pathway, inhibiting DKK1, and/or activating a noggin pathway. In some such embodiment, the BMP pathway is inhibited with a BMP inhibitor (e.g., a BMP4 inhibitor such as Noggin). In some such embodiments DKK1 is inhibited with a DKK1 inhibitor. Thus, in one embodiment, the present disclosure comprises activating the Wnt pathway with a Wnt agonist and a TGF-beta inhibitor. In one embodiment, the present disclosure comprises activating the Wnt pathway with a GSK3-alpha inhibitor and a TGF-beta inhibitor. In one embodiment, the present disclosure comprises activating the Wnt pathway with a GSK3-beta inhibitor and a TGF-beta inhibitor. In various embodiments such methods further comprise contacting the cells with a Notch activator, HDAC inhibitor, a BMP4 antagonist, upregulator of Sox2, Vitamin D (calcitriol), Vitamin B (nicotinomide), Vitamin A, Vitamin C (pVc), Lgr4, p38/MAPK inhibitor, ROCK inhibitor, TGF-beta RI kinase inhibitor, and/or an inhibitor of Alk2, Alk4, Alk5, and/or Alk7.

In certain embodiments, therefore, the present disclosure provides compositions and methods to induce self-renewal of a population of supporting cells by activating pathways and mechanisms that are involved in inducing stem cell properties, such as those used to create “induced pluripotent stem cells”. In some embodiments, these pathways are activated with small molecules. For example, a compound when applied in vitro to a supporting cell population may induce the population to proliferate to a high degree and in high purity in a Stem Cell Proliferation Assay, and also allow the population to differentiate into a high purity population of a tissue cells in a Stem Cell Differentiation Assay. In one such embodiment, a compound or composition of the present disclosure induces and maintains stem cell properties by proliferating to produce stem cells that can divide for many generations and maintain the ability to have a high proportion of the resulting cells differentiate into tissue cells. Further, the proliferating stem cells express stem cell markers which may include one or more of Lgr5, Sox2, Sox9, Opem1, Phex, lin28, Lgr6, cyclin D1, Msx1, Myb, Kit, Gdnf3, Zic3, Dppa3, Dppa4, Dppa5, Nanog, Esrrb, Rex1, Dnmt3a, Dnmt3b, Dnmt31, Utf1, Tcl1, Oct4, Klf4, Pax6, Six2, Zic1, Zic2, Otx2, Bmi1, CDX2, STAT3, Smad1, Smad2, Smad2/3, Smad4, Smad5, and Smad7.

In certain embodiments, the disclosure provides a method for expanding a population of vestibular cells in a vestibular tissue comprising contacting the vestibular tissue with (i) a Wnt agonist, GSK3-alpha inhibitor, or GSK3-beta inhibitor (or a derivative or pharmaceutically-acceptable salt thereof) (ii) in combination with a TGF-beta inhibitor (or a derivative or pharmaceutically-acceptable salt thereof), thereby facilitating generation of supporting cells and/or inner ear hair cells from the expanded population of stem cells. In certain embodiments, the disclosure provides a method for increasing the cell density of supporting cells in a population of vestibular cells; the method comprising activating supporting cells with a Wnt agonist, GSK3-alpha inhibitor, or GSK3-beta inhibitor in combination with a TGF-beta inhibitor, to induce stem cell properties, proliferating the activated supporting cells (while maintaining the multi-potent character of the supporting cells in the newly formed daughter cells), and differentiating the proliferated supporting cells into hair cells to form an expanded vestibular cell population. In some embodiments, the cell density of hair cells in the expanded vestibular cell population exceeds the cell density of hair cells in the original (non-expanded) vestibular cell population. In some embodiments, the supporting cell population is an in vitro supporting cell and hair cells population. In some embodiments, the supporting cell and hair cell population is an in vivo cell population. In some embodiments, the proliferation stage is controlled to substantially maintain the native organization of the vestibular structure. In certain embodiments the supporting cell population includes supporting cells that are endogenous to the Vestibular Organs. Thus, in one embodiment, the disclosure provides a method for expanding a population of vestibular cells in a vestibular tissue comprising contacting the vestibular tissue with a Wnt agonist and a TGF-beta inhibitor. In one embodiment, the disclosure provides a method for expanding a population of vestibular cells in a vestibular tissue comprising contacting the vestibular tissue with a GSK3-alpha inhibitor and a TGF-beta inhibitor. In one embodiment, the disclosure provides a method for expanding a population of vestibular cells in a vestibular tissue comprising contacting the vestibular tissue with a GSK3-beta inhibitor and a TGF-beta inhibitor

In certain embodiments, the method produces stem cells in a Stem Cell Proliferation Assay that express stem cells markers, Sox2, Sox9, Pax2, Pax6, Pax8, Bmi1, Lgr5⁺. In certain embodiments, if a mixed population of supporting and non-supporting stem cells are placed in a Stem Cell Proliferation Assay, the method increases the fraction of cells in the population that express supporting cell markers.

Expanding supporting cell populations to a degree that destroys the native organization of the vestibular structure could inhibit vestibular function. Driving proliferation of existing supporting cells with a small molecule signal may allow for a more controlled regeneration of hair cells than using gene delivery, which is incapable of targeting a specific cell type and permanently alters a cell's genetic information. An approximately normal vestibular structure is desired with rows of hair cells that have supporting cells between them, and hair cells do not contact other hair cells. Further, it would be desirable to avoid using genetic modification to drive proliferation to create large cell aggregations in the vestibular organs that disrupt the organ's anatomy. In embodiments, the proliferation of the stem cells (e.g., that which is induced by a Wnt agonist, a GSK3-alpha inhibitor, or a GSK3-beta inhibitor) is enhanced by adding a modulator of cell cycle regulators of a TGF-beta pathway. In embodiments, proliferation of the stem cells (e.g., that which is induced by a Wnt agonist, a GSK3-alpha inhibitor, or a GSK3-beta inhibitor) is enhanced by adding a TGF-beta inhibitor (e.g., one or more of the TGF-beta inhibitors known in the art or described herein) or optionally a DKK1 inhibitor or a BMP antagonist such as, e.g., a BMP4 antagonist (e.g., Noggin).

In certain embodiments, the disclosure provides a method for increasing the cell density of hair cells in an initial population of vestibular cells comprising hair cells and supporting cells, the method comprising selectively expanding the number of supporting cells in the initial population to form an intermediate vestibular cell population wherein the ratio of the number of supporting cells to hair cells in the intermediate vestibular cell population exceeds the ratio of the number of supporting cells to hair cells in the initial vestibular cell population. In some embodiments, the supporting cells expansion is facilitated by contacting the supporting cells with (i) a Wnt agonist, a GSK3-alpha inhibitor, or a GSK3-beta inhibitor and (ii) a TGF-beta inhibitor. In certain embodiments, the method further comprises generating hair cells in the intermediate vestibular cell population to form an expanded vestibular cell population wherein the ratio of the number of hair cells to supporting cells in the expanded vestibular cell population exceeds the ratio of the number of hair cells to supporting cells in the intermediate vestibular cell population. Thus, in some embodiments, the supporting cells expansion is facilitated by contacting the supporting cells with a Wnt agonist and a TGF-beta inhibitor. In some embodiments, the supporting cells expansion is facilitated by contacting the supporting cells with a GSK3-alpha inhibitor and a TGF-beta inhibitor. In some embodiments, the supporting cells expansion is facilitated by contacting the supporting cells with a GSK3-beta inhibitor and a TGF-beta inhibitor.

In certain embodiments, the disclosure provides a method for increasing the number of supporting cells or increasing the stem cell/supporting cell activity in an initial population of vestibular cells, wherein the initial population comprises supporting cells and hair cells. For example, in one such method an intermediate population is formed in which the number of supporting cells is expanded relative to the initial population. Alternatively, in one such method an intermediate population is formed in which the sternness of the supporting cells relative to the initial population is increased. Alternatively, a method where the number of supporting cells is increased relative to the initial cell population by activating stem cell gene expression in cell types that normally lack or have very low levels of stem cell gene expression. By way of further example, an intermediate population is formed in which the number of supporting cells is expanded and the Wnt activity is increased relative to the initial vestibular cell population. Thereafter, hair cells in the intermediate vestibular cell population may be generated to form an expanded vestibular cell population wherein the ratio of hair cells to supporting cells in the expanded vestibular cell population exceeds the ratio of the number of hair cells to supporting cells in the intermediate vestibular cell population. In some such embodiments, the Wnt activity is increased by contacting the supporting cells with a Wnt agonist, a GSK3-alpha inhibitor, or a GSK3-beta inhibitor. In embodiments, such methods further comprise contacting the supporting cells with a TGF-beta inhibitor.

In some embodiments, sternness is induced by activating the Wnt pathway (e.g., with a Wnt agonist, a GSK3-alpha inhibitor, or a GSK3-beta inhibitor). In embodiments, the induction of sternness further comprises inhibiting TGF-beta.

In certain embodiments, the disclosure provides a method for generating hair cells, the method comprising: administering to a stem cell population a composition comprising (i) a Wnt agonist, a GSK3-alpha inhibitor, or a GSK3-beta inhibitor (or a derivative or pharmaceutically-acceptable salt thereof), and (ii) a TGF-beta inhibitor (or a derivative or pharmaceutically-acceptable salt thereof), thereby proliferating stem cells in the stem cell population and resulting in an expanded population of stem cells that can generate into inner ear hair cells.

In certain embodiments, the disclosure provides compositions, systems, and methods for preventing and treating balance dysfunction, such as vertigo, dizziness, benign paroxysmal positional vertigo (BPPV), labyrinthitis or vestibular neuritis, Meniere's disease, and ototoxicity. For example, in certain embodiments, the disclosure provides methods for preventing or treating balance impairments in a subject comprising administering to said subject an effective amount of a composition comprising a (i) Wnt agonist, a GSK3-alpha inhibitor, or a GSK3-beta inhibitor and (ii) a TGF-beta inhibitor. In some embodiments, the (i) Wnt agonist, GSK3-alpha inhibitor, or GSK3-beta inhibitor and the (ii) TGF-beta inhibitor are administered separately, e.g., sequentially.

In certain embodiments, the present disclosure also relates to ex-vivo uses of cells described herein. For example, approaches described herein can be used for high throughput screening and for discovery purposes. For example, certain embodiments of the present disclosure are useful for identifying agents that proliferate hair cell progenitors and/or increase numbers of hair cells, and also agents that protect supporting cells and/or hair cells (e.g. to support their survival), and also for identifying agents that are toxic or not toxic to supporting cells or differentiated progeny including hair cells.

In certain embodiments, the disclosure provides for methods for inhibiting the loss or death of the cells of the auditory system in a subject comprising administering to said subject an effective amount of a composition described herein or derivative thereof or pharmaceutically-acceptable salt thereof and an acceptable carrier or excipient, thereby inhibiting loss or death of the cells of the auditory system in the subject.

In certain embodiments, the disclosure provides methods for maintaining or promoting the growth of cells of the auditory system in a subject comprising administering to said subject a composition comprising as an agent described herein or derivative thereof or pharmaceutically-acceptable salt thereof and an acceptable carrier or excipient in an effective amount so as to augment or initiate endogenous repair, thereby maintaining or promoting the growth of cells of the auditory system in the subject.

Also described herein is a method for expanding a population of vestibular cells in a vestibular tissue comprising a parent population of cells, the parent population including supporting cells and a number of supporting cells, the method comprising contacting the vestibular tissue with a stem cell proliferator to form an expanded population of cells in the vestibular tissue, wherein the stem cell proliferator is capable (i) in a stem cell proliferation assay of increasing the number of supporting cells in a stem cell proliferation assay cell population by a factor of at least 10 and (ii) in a stem cell differentiation assay of forming hair cells from a cell population comprising supporting cells. In certain embodiments, the stem cell proliferator is a Wnt agonist, GSK3-alpha inhibitor, or GSK3-beta inhibitor. In certain embodiments, the method further comprises contacting the vestibular tissue with a TGF-beta inhibitor. Thus, in various embodiments, the present disclosure provides a method for expanding a population of vestibular cells in a vestibular tissue comprising a parent population of cells, the parent population including supporting cells and a number of supporting cells, the method comprising contacting the vestibular tissue with a Wnt agonist and a TGF-beta inhibitor. In some embodiments, the method comprising contacting the vestibular tissue with a GSK3-alpha inhibitor and a TGF-beta inhibitor. In some embodiments, the method comprises contacting the supporting cells with a GSK3-beta inhibitor and a TGF-beta inhibitor.

Also described herein is a method for expanding a population of vestibular cells in a vestibular tissue comprising a parent population of cells, the parent population including supporting cells, the method comprising contacting the vestibular tissue with a stem cell proliferator (e.g., (i) a Wnt agonist, a GSK3-alpha inhibitor, or a GSK3-beta inhibitor and (ii) a TGF-beta inhibitor) to form an expanded population of cells in the vestibular tissue. The stem cell proliferator can be capable of (i) forming a proliferation assay final cell population from a proliferation assay initial cell population over a proliferation assay time period in a stem cell proliferation assay and (ii) forming a differentiation assay final cell population from a differentiation assay initial cell population over a differentiation assay time period in a stem cell differentiation assay wherein: (a) the proliferation assay initial cell population has (i) a proliferation assay initial number of total cells, (ii) a proliferation assay initial number of supporting cells, (iii) a proliferation assay initial number of hair cells, (iv) a proliferation assay initial supporting cell fraction that equals the ratio of the proliferation assay initial number of supporting cells to the proliferation assay initial number of total cells, and (v) a proliferation assay initial hair cell fraction that equals the ratio of the proliferation assay initial number of hair cells to the proliferation assay initial number of total cells; (b) the proliferation assay final cell population has (i) a proliferation assay final number of total cells, (ii) a proliferation assay final number of supporting cells, (iii) a proliferation assay final number of hair cells, (iv) a proliferation assay final supporting cell fraction that equals the ratio of the proliferation assay final number of supporting cells to the proliferation assay final number of total cells and (v) a proliferation assay final hair cell fraction that equals the ratio of the proliferation assay final number of hair cells to the proliferation assay final number of total cells; (c) the differentiation assay initial cell population has (i) a differentiation assay initial number of total cells, (ii) a differentiation assay initial number of supporting cells, (iii) a differentiation assay initial number of hair cells, (iv) a differentiation assay initial supporting cell fraction that equals the ratio of the differentiation assay initial number of supporting cells to the differentiation assay initial number of total cells, and (v) a differentiation assay initial hair cell fraction that equals the ratio of the differentiation assay initial number of hair cells to the differentiation assay initial number of total cells; (d) the differentiation assay final cell population has (i) a differentiation assay final number of total cells, (ii) a differentiation assay final number of supporting cells, (iii) a differentiation assay final number of hair cells, (iv) a differentiation assay final supporting cell fraction that equals the ratio of the differentiation assay final number of supporting cells to the differentiation assay final number of total cells, and (v) a differentiation assay final hair cell fraction that equals the ratio of the differentiation assay final number of hair cells to the differentiation assay final number of total cells; (e) the proliferation assay final number of supporting cells exceeds the proliferation assay initial number of supporting cells by a factor of at least 10; and (f) the differentiation assay final number of hair cells is a non-zero number.

The proliferation assay final number of supporting cells can be greater than the proliferation assay initial number of supporting cells by a factor of at least 50, or by a factor of at least 100. The expanded population of cells in the vestibular tissue can include a greater number of hair cells than does the parent population. The proliferation assay final supporting cell fraction can be greater than the differentiation assay initial supporting cell fraction by at least a factor of 2. The differentiation assay final hair cell fraction can be greater than the proliferation assay initial hair cell fraction by at least a factor of 2. The proliferation assay final hair cell fraction can be at least 25% less than the proliferation assay initial hair cell fraction. The proliferation assay final supporting cell fraction can be at least 10% greater than proliferation assay initial supporting cell fraction. One of more morphological characteristics of the vestibular tissue can be maintained. Native morphology can be maintained. The stem cell proliferator can be dispersed in a biocompatible matrix, which can be a biocompatible gel or foam. The vestibular tissue can be an in vivo vestibular tissue or an ex vivo vestibular tissue. The method can produce a population of supporting cells that are in s-phase. The vestibular tissue can be in a subject, and contacting the vestibular tissue with the composition can be achieved by administering the composition transtympanically to the subject. Contacting the vestibular tissue with the composition can result in improved balance functioning of the subject.

In some embodiments, the method includes contacting a population of vestibular cell with (i) a Wnt agonist, a GSK3-alpha inhibitor, or a GSK3-beta inhibitor and (ii) an agent that induces Sox2 activity. In some embodiments, the agent that induces Sox2 activity is a TGF-beta inhibitor. In some embodiments, the TGF-beta inhibitor is 616452 (RepSox).

Also described herein is a method for increasing Sox2 activity in a population of vestibular cells in a vestibular tissue comprising a parent population of cells, the parent population including supporting cells, the method comprising contacting the vestibular tissue with a TGF-beta inhibitor (e.g., 616452) to increase Sox2 activity.

Also described herein is a method of treating a subject who has, or is at risk of developing, balance impairment, wherein the method include transtympanically administering to a vestibular tissue of the subject a composition comprising at least one agent to increase Sox2 activity.

Also described herein is a method for expanding a population of vestibular cells in a vestibular tissue comprising a parent population of cells, the parent population including supporting cells, the method comprising contacting the vestibular tissue with a stem cell proliferator (e.g., a Wnt agonist, a GSK3-alpha inhibitor, or a GSK3-beta inhibitor) and an agent to induce Sox2 activity, to form an expanded population of cells in the vestibular tissue. In some embodiments, the agent to induce Sox2 is 616452/(RepSox). Thus, in various embodiments, the present disclosure provides a method for expanding a population of vestibular cells in a vestibular tissue comprising a parent population of cells, the parent population including supporting cells, the method comprising contacting the vestibular tissue with a Wnt agonist and a TGF-beta inhibitor. In some embodiments, the present disclosure provides a method for expanding a population of vestibular cells in a vestibular tissue comprising a parent population of cells, the parent population including supporting cells, the method comprising contacting the vestibular tissue with a GSK3-alpha inhibitor and a TGF-beta inhibitor. In some embodiments the present disclosure provides a method for expanding a population of vestibular cells in a vestibular tissue comprising a parent population of cells, the parent population including supporting cells, the method comprising contacting the vestibular tissue with a GSK3-beta inhibitor and a TGF-beta inhibitor.

Also described herein is a method of treating a subject who has, or is at risk of developing, balance impairment. The method can include transtympanically administering to a vestibular tissue of the subject a composition comprising at least one stem cell proliferator. The at least one stem cell proliferator can include at least one of a stemness driver (e.g., a Wnt agonist, a GSK3-alpha inhibitor, or a GSK3-beta inhibitor) and a modulator of pathways that regulate cell cycle or plasticity (e.g., a TGF-beta inhibitor). The at least one stem cell proliferator can include both a stemness driver (e.g., a Wnt agonist, a GSK3-alpha inhibitor, or a GSK3-beta inhibitor) and a modulator of pathways that regulate cell cycle or plasticity (e.g., a TGF-beta inhibitor).

In some embodiments, the methods of the present disclosure further comprise contacting the vestibular tissue with epidermal growth factor, basic fibroblast growth factor, insulin-like growth factor 1, and 616452 either individually or in combination (e.g., in addition to contacting with a Wnt agonist, a GSK3-alpha inhibitor, or a GSK3-beta inhibitor). Also described herein is a method of generating Myo7a+ vestibular cells. The method can include contacting supporting vestibular cells with a composition comprising a stemness driver (e.g., Wnt agonist, GSK3-alpha inhibitor, or GSK3-beta inhibitor) and a TGF-beta inhibitor, thereby generating an expanded population of supporting cells that can differentiate into Myo7a+ vestibular cells.

Certain embodiments relate to pharmaceutical compositions, comprising a pharmaceutically-acceptable carrier and (i) a Wnt agonist, a GSK3-alpha inhibitor, or a GSK3-beta inhibitor and (ii) a TGF-β inhibitor, or a pharmaceutically-acceptable salt thereof. In some embodiments, the composition is adapted for administration to the inner ear and/or middle ear. In some instances, the composition is adapted for local administration to the round window membrane. In some embodiments, the composition is adapted for intratympanic or transtympanic administration, for example, to vestibular tissue.

In some embodiments, (i) and (ii) are dispersed in a biocompatible matrix. In certain embodiments, the biocompatible matrix is a biocompatible gel or foam.

In some embodiments, the Wnt agonist, GSK3-alpha inhibitor, or GSK3-beta inhibitor is selected from CHIR99021, LY2090314, AZD1080, or GSK3 inhibitor XXII.

In certain embodiments, the TGF-β Inhibitor is selected from 616452 (Repsox), Galunisertib (LY2157299), EW-719, IN-1130, EW-7203, EW-7195, SM16, R 268712, GW788388, SB-431542, A 83-01, and PF-03671148.

Particular compositions further comprise an additional agent selected from a Notch activator, HDAC inhibitor, a BMP4 antagonist, Noggin (Inhibits BMP4), Sox2, Vitamin D (calcitriol), Vitamin B (nicotinomide), Vitamin A, Vitamin C (pVc). Lgr4, p38/MAPK inhibition, ROCK inhibition, and/or Alk4/7 inhibition.

Some compositions further comprise an epidermal growth factor (EGF), fibroblast growth factor (FGF), insulin-like growth factor (IGF), or a combination thereof.

In some embodiments, the pharmaceutical composition comprises a poloxamer. In particular embodiments, the poloxamer comprises at least one of Poloxamer 188 and Poloxamer 407 or mixtures thereof. In some embodiments, the poloxamer is in a concentration between about 5 wt % and about 25 wt % relative to the composition. In particular embodiments, the poloxamer is in a concentration between about 10 wt % and about 23 wt % relative to the composition. In some embodiments, the poloxamer is in a concentration between about 15 wt % and about 20 wt % relative to the composition. In specific embodiments, the poloxamer is in a concentration is approximately 17 wt % relative to the composition.

In certain compositions, the Wnt agonist, GSK3-alpha inhibitor, or GSK3-beta inhibitor is at a concentration of about 0.01 uM to 1000 mM, about 0.1 uM to 1000 mM, about 1 uM to 100 mM, about 10 uM to 10 mM, about 1 uM to 10 uM, about 10 uM to 100 uM, about 100 uM to 1000 uM, about 1 mM to 10 mM, or about 10 mM to 100 mM; or at a concentration ratio of about 0.01 to 1,000,000 fold relative to its Effective Stemness Driver Concentration, or about 0.1 to 100,000 fold relative to its Effective Stemness Driver Concentration, or about 1 to 10,000 fold relative to its Effective Stemness Driver Concentration, or about 100 to 5000 fold relative to its Effective Sternness Driver Concentration, or about 50 to 2000 fold relative to its Effective Sternness Driver Concentration, or about 100 to 1000 fold relative to its Effective Sternness Driver Concentration, or at about 1000 fold relative to its Effective Sternness Driver Concentration; or at a concentration of about 0.01 nM to 1000 uM, about 0.1 nM to 1000 uM, about 1 nM to 100 uM, about 10 nM to 10 uM, about 1 nM to 10 nM, about 10 nM to 100 nM, about 100 nM to 1000 nM, about 1 uM to 10 uM, or about 10 uM to 100 uM.

In some embodiments, the Wnt agonist, GSK3-alpha inhibitor, or GSK3-beta inhibitor is CHIR99021, which is at a concentration of about 1 uM to 1000 mM, about 10 uM to 100 mM, about 100 uM to 100 mM, about 1 mM to 10 mM, or about 1, 2, 3, 4, 5, 6, 7, 8, 9, or 10 mM; or at a concentration of about 1 nM to 1000 uM, about 10 nM to 100 uM, about 100 nM to 100 uM, about 1 uM to 10 uM, or about 1, 2, 3, 4, 5, 6, 7, 8, 9, or 10 uM.

In some embodiments, the Wnt agonist, GSK3-alpha inhibitor, or GSK3-beta inhibitor is LY2090314, which is at a concentration of about 0.01 uM to 1000 mM, about 0.1 uM to 10 mM, about 1 uM to 1 mM, about 10 uM, about 20 uM, about 30 uM, about 40 uM, or about 50 uM; or at a concentration of about 0.01 nM to 1000 uM, about 0.1 nM to 10 uM, about 1 nM to 1 uM, about 1 nM to 100 nM, or about 10 nM.

In some embodiments, the Wnt agonist, GSK3-alpha inhibitor, or GSK3-beta inhibitor is AZD1080, which is at a concentration of about 0.1 uM to 1000 mM, about 1 uM to 1000 mM, about 10 uM to 100 mM, about 100 uM to 10 mM, about 1 mM to 10 mM, or about 1, 2, 3, 4, 5, 6, 7, 8, 9, or 10 mM; or at a concentration of about 1 nM to 1000 uM, about 10 nM to 1000 uM, about 100 nM to 100 uM, about 1 uM to 10 uM, or about 1, 2, 3, 4, 5, 6, 7, 8, 9, or 10 uM.

In some embodiments, the Wnt agonist, GSK3-alpha inhibitor, or GSK3-beta inhibitor is GSK3 inhibitor XXII, which is at a concentration of about 0.1 uM to 1000 mM, about 1 uM to 100 mM, about 10 uM to 10 mM, about 100 uM to 10 mM, about 100 uM to 1 mM, or about 1, 2, 3, 4, 5, 6, 7, 8, 9, or 10 mM; or at a concentration of about 0.1 nM to 1000 uM, about 1 nM to 100 uM, about 10 nM to 10 uM, about 100 nM to 1 uM, or about 0.5 uM.

In some embodiments, the TGF-beta inhibitor is at a concentration of about 0.01 uM to 1000 mM, about 0.1 uM to 1000 mM, about 1 uM to 100 mM, about 0.1 uM to 1 uM, about 1 uM to 10 uM, about 10 uM to 100 uM, about 100 uM to 1 mM, about 1 mM to 10 mM, or about 100 mM to 1000 mM, or about 10 mM to 100 mM, or about 100 mM to 1000 mM; or at a concentration ratio of about 0.1 to 1,000,000 fold relative to its Effective TGF-beta Concentration, or about 1 to 100,000 fold relative to its Effective TGF-beta Concentration, or about 10 to 10,000 fold relative to its Effective TGF-beta Concentration, or about 100 to 1000 fold relative to its Effective TGF-beta Concentration, or about 1000 fold relative to its Effective TGF-beta Concentration; or at a concentration of about 0.01 nM to 1000 uM, or about 0.1 nM to 1000 uM, about 1 nM to 100 uM, about 10 nM to 10 uM, about 1 nM to 10 nM, about 10 nM to 100 nM, about 100 nM to 1000 nM, about 1 uM to 10 uM, about 10 uM to 100 uM, or about 100 uM to 1000 uM.

In some embodiments, the TGF-beta inhibitor is 616452 (Repsox) at a concentration of about 1 uM to 1000 mM, or about 10 uM to 1000 mM, or about 100 uM to 10 mM, or about 2 mM; or at a concentration of about 1 nM to 1000 uM, about 10 nM to 100 uM, about 100 nM to 10 uM, or about 2 uM.

In some embodiments, the BMP4 antagonist is at a concentration of about 0.01 uM to 1000 mM, 0.1 uM to 1000 mM, about 1 uM to 100 mM, about 10 uM to 10 mM, about 0.1 uM to 1 uM, about 1 uM to 10 uM, about 10 uM to 100 uM, about 100 uM to 1 mM, about 1 mM to 10 mM, about 10 mM to 100 mM, about 100 mM to 1000 mM; or at a concentration ratio of about 0.1 to 1,000,000 fold relative to its Effective BMP4 Antagonist Concentration, or about 1 to 100,000 fold relative to its Effective BMP4 Antagonist Concentration, or about 10 to 10,000 fold relative to its Effective BMP4 Antagonist Concentration, or about 100 to 1000 fold relative to its Effective BMP4 Antagonist Concentration, or about 1000 fold relative to its Effective BMP4 Antagonist Concentration; or at a concentration of about 0.01 nM to 100 uM, about 1 nM to 100 uM, about 10 nM to 10 uM, about 1 nM to 10 nM, about 10 nM to 100 nM, about 100 nM to 1000 nM, about 1 uM to 10 uM, about 10 uM to 100 uM, or about 100 uM to 1000 uM.

In some embodiments, the BMP4 antagonist is DMH1 at a concentration of about 1 uM to 1000 mM, about 10 uM to 100 mM, about 100 uM to 10 mM, or about 1 mM; or at a concentration of about 1 nM to 1000 uM, or about 10 nM to 100 uM, about 100 nM to 10 uM, or about 1 uM.

In some embodiments, the BMP4 antagonist is Noggin at a concentration of about 1 ug/ml to 10,000 ug/ml, about 10 ug/ml to 1000 ug/ml, or about 100 ug/ml; or at a concentration of about 1 ng/ml to 10,000 ng/ml, about 10 ng/ml to 1000 ng/ml, or about 100 ng/ml.

In some embodiments, the HDAC inhibitor is at a concentration of about 0.01 uM to 100,000 mM, about 1 uM to 10,000 mM, about 10 uM to 10,000 mM, about 100 uM to 1000 mM, about 1 uM to 10 uM, about 10 uM to 100 uM, about 100 uM to 1000 uM, about 1000 uM to 10 mM, about 10 mM to 100 mM, about 100 mM to 1000 mM, or about 1000 mM to 10,000 mM; or at a concentration ratio of about 0.1 to 1,000,000 fold relative to its Effective Concentration, or about 1 to 100,000 fold relative to its Effective Concentration, or about 10 to 10,000 fold relative to its Effective Concentration, or about 100 to 1000 fold relative to its Effective Concentration, or about 1000 fold relative to its Effective Concentration; or at a concentration of about 0.01 nM to 100,000 uM, about 1 nM to 10,000 uM, about 10 nM to 10,000 uM, about 100 nM to 1000 uM, about 1 nM to 10 nM, about 10 nM to 100 nM, about 100 nM to 1000 nM, about 1 uM to 10 uM, about 10 uM to 100 uM, about 100 uM to 1000 uM, or about 1000 uM to 10,000 uM.

In some embodiments, the HDAC inhibitor is valproic acid at a concentration of about 10 uM to 100,000 mM, about 1 mM to 10,000 mM, about 10 mM to 10,000 mM, about 100 mM to 10,000 mM, about 200 mM to 2000 mM, about 1000 mM, or about 600 mM; or at a concentration of about 10 nM to 100,000 uM, 1 uM to 10,000 uM, about 10 uM to 10,000 uM, about 100 uM to 10,000 uM, about 200 uM to 2000 uM, or about 1000 uM.

In certain embodiments, the Effective Sternness Driver Concentration, Effective TGF-beta Concentration, Effective BMP4 Antagonist Concentration, and/or the Effective Concentration is measured in an Lgr5 proliferation assay, as described herein.

The pharmaceutical compositions can be used in any one or more of the methods described herein, including the use of the compositions for expanding a population of vestibular cells in a vestibular tissue. The pharmaceutical compositions can also be used for treating a subject who has, or is at risk of developing, a disease associated with absence or lack of vestibular cells, for example, Type I vestibular hair cell and/or Type II vestibular hair cells. The pharmaceutical compositions can also be used for treating a subject who has, or is at risk of developing, a vestibular condition.

Other objects and features will be in part apparent and in part pointed out hereinafter.

### DETAILED DESCRIPTION

### Definitions

In this application, the use of “or” means “and/or” unless stated otherwise. As used in this application, the term “comprise” and variations of the term, such as “comprising” and “comprises,” are not intended to exclude other additives, components, integers or steps. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Administration” refers to introducing a substance into a subject. In some embodiments, administration is auricular, intraauricular, intravestibular, intravestibular, or transtympanically, e.g., by injection. In some embodiments, administration is directly to the inner ear, e.g. injection through the round window, otic capsule, or vestibular canals. In some embodiments, administration is directly into the inner ear via a vestibular implant delivery system. In some embodiments, the substance is injected transtympanically to the middle ear. In certain embodiments “causing to be administered” refers to administration of a second component after a first component has already been administered (e.g., at a different time and/or by a different actor). In embodiments, the Wnt agonist, GSK3-alpha inhibitor, or GSK3-beta inhibitor and the TGF-beta inhibitor of the present disclosure are administered to a subject as a single composition (e.g., pharmaceutical composition) comprising both the Wnt agonist, GSK3-alpha inhibitor, or GSK3-beta inhibitor and the TGF-beta inhibitor. In some embodiments, the Wnt agonist, GSK3-alpha inhibitor, or GSK3-beta inhibitor and the TGF-beta inhibitor are administered to the subject separately, e.g., sequentially.

An “antibody” refers to an immunoglobulin polypeptide, or fragment thereof, having immunogen binding ability.

As used herein, an “agonist” is an agent that causes an increase in the expression or activity of a target gene, protein, or a pathway, respectively. Therefore, an agonist can bind to and activate its cognate receptor in some fashion, which directly or indirectly brings about this physiological effect on the target gene or protein. An agonist can also increase the activity of a pathway through modulating the activity of pathway components, for example, through inhibiting the activity of negative regulators of a pathway. Therefore, a “Wnt agonist” can be defined as an agent that increases the activity of Wnt pathway, which can be measured by increased TCF/LEF-mediated transcription in a cell. Therefore, a “Wnt agonist” can be a true Wnt agonist that binds and activates a Frizzled receptor family member, including any and all of the Wnt family proteins, an inhibitor of intracellular beta-catenin degradation, and activators of TCF/LEF.

An “antagonist” refers to an agent that binds to a receptor, and which in turn decreases or eliminates binding by other molecules.

“Antisense” refers to a nucleic acid sequence, regardless of length, that is complementary to the coding strand or mRNA of a nucleic acid sequence. Antisense RNA can be introduced to an individual cell, tissue or organoid. An anti-sense nucleic acid can contain a modified backbone, for example, phosphorothioate, phosphorodithioate, or other modified backbones known in the art, or may contain non-natural internucleoside linkages.

As referred to herein, a “complementary nucleic acid sequence” is a nucleic acid sequence capable of hybridizing with another nucleic acid sequence comprised of complementary nucleotide base pairs. By “hybridize” is meant pair to form a double-stranded molecule between complementary nucleotide bases (e.g., adenine (A) forms a base pair with thymine (T), as does guanine (G) with cytosine (C) in DNA) under suitable conditions of stringency. (See, e.g., Wahl, G. M. and S. L. Berger (1987) Methods Enzymol. 152:399; Kimmel, A. R. (1987) Methods Enzymol. 152:507).

“Auricular administration” refers to a method of using a catheter or wick device to administer a composition across the tympanic membrane to the inner ear of the subject. To facilitate insertion of the wick or catheter, the tympanic membrane may be pierced using a suitably sized syringe or pipette. The devices could also be inserted using any other methods known to those of skill in the art, e.g., surgical implantation of the device. In particular embodiments, the wick or catheter device may be a stand-alone device, meaning that it is inserted into the ear of the subject and then the composition is controllably released to the inner ear. In other particular embodiments, the wick or catheter device may be attached or coupled to a pump or other device that allows for the administration of additional compositions. The pump may be automatically programmed to deliver dosage units or may be controlled by the subject or medical professional.

“Biocompatible Matrix” as used herein is a polymeric carrier that is acceptable for administration to humans for the release of therapeutic agents. A Biocompatible Matrix may be a biocompatible gel or foam.

“Cell Aggregate” as used herein shall mean a body cells in the Vestibular Organs that have proliferated to form a cluster of a given cell type that is greater than 40 microns in diameter and/or produced a morphology in which greater than 3 cell layers reside perpendicular to the basement membrane. A “Cell Aggregate” can also refer a process in which cell division creates a body of cells that cause one or more cell types to breach the reticular lamina, or the boundary between endolymph and perilymph

“Cell Density” as used herein in connection with a specific cell type is the mean number of that cell type per area in a Representative Microscopy Sample. The cell types may include but are not limited to supporting cells, hair cells, or supporting cells. The Cell Density may be assessed with a given cell type in a given organ or tissue, including but not limited to the cochlea or Vestibular Organs. For instance, the supporting Cell Density in the Vestibular Organs is the Cell Density of supporting cells as measured across the Vestibular Organs. Typically, supporting cells and supporting cells will be enumerated by taking cross sections of the Vestibular Organs. Typically, hair cells will be enumerated by looking down at the surface of the Vestibular Organs, though cross sections may be used in some instances, as described in a Representative Microscopy Sample. Typically, Cell Density of supporting cells will be measured by analyzing whole mount preparations of the Vestibular Organs and counting the number of supporting cells across a given distance along the surface of the epithelia, as described in a Representative Microscopy Sample. Hair cells may be identified by their morphological features such as bundles or hair cell specific stains (e.g., Myosin VIIa, Oncomodulin, vGlut3, Pou4f3, Espin, conjugated-Phalloidin, PMCA2, Ribeye, Atoh1, etc.). supporting cells may be identified by specific stains or antibodies (e.g. Sox9-GFP transgenic reporter, anti-Sox9 antibody, etc.)

“Vestibular Concentration” as used herein will be the concentration of a given agent as measured through sampling inner ear fluid (endolymph and/or perilymph). Unless otherwise noted, the sample should contain a substantial enough portion of the inner ear fluid so that it is approximately representative of the average concentration of the agent in the inner ear. For example, samples may be drawn from a vestibular canal, and/or round window, and/or oval window, and a series of fluid samples drawn in series such that individual samples are comprised of vestibular fluid in specified portions of the inner ear.

“Complementary nucleic acid sequence” refers to a nucleic acid sequence capable of hybridizing with another nucleic acid sequence comprised of complementary nucleotide base pairs.

“Cross-Sectional Cell Density” as used herein in connection with a specific cell type is the mean number of that cell type per area of cross section through a tissue in a Representative Microscopy Sample. Cross sections of the Vestibular Organs can also be used to determine the number of cells in a given plane. Typically, hair cells Cross-sectional Cell Density will be measured by analyzing whole mount preparations of the Vestibular Organs and counting the number of hair cells across a given distance in cross sections taken along a portion of the epithelia, as described in a Representative Microscopy Sample. Typically, Cross-sectional Cell Density of supporting cells will be measured by analyzing whole mount preparations of the Vestibular Organs and counting the number of supporting cells across a given distance in cross sections taken along a portion of the epithelia, as described in a Representative Microscopy Sample. Hair cells may be identified by their morphological features such as bundles or hair cell specific stains (suitable stains include e.g., Myosin VIIa, vGlut3, Pou4f3, conjugated-Phalloidin, PMCA2, Atoh1, Oncomodulin, Sox2, etc.). supporting cells may be identified by specific stains or antibodies (suitable stains and antibodies include fluorescence in situ hybridization of Lgr5 mRNA, Sox9 transgenic reporter system, anti-Sox9 antibodies, etc.).

“Decreasing” refers to decreasing by at least 5%, for example, 5, 6, 7, 8, 9, 10, 15, 20, 25, 30, 35, 40, 45, 50, 55, 60, 65, 70, 75, 80, 85, 90, 95, 99 or 100%, for example, as compared to the level of reference.

“Decreases” also means decreases by at least 1-fold, for example, 1, 2, 3, 4, 5, 6, 7, 8, 9, 10, 15, 20, 30, 40, 50, 60, 70, 80, 90, 100, 200, 500, 1000-fold or more, for example, as compared to the level of a reference.

“Differentiation Period” as used herein is the duration of time in which growth factors, an Effective Sternness Driver Concentration, and an Effective TGF-beta Inhibitor Concentration are removed after a Stem Cell Proliferation Assay. In some instances, a gamma secretase inhibitor and/or Wnt agonist may be added without growth factors.

“Effective Concentration” may be the Effective Sternness Driver Concentration for a Sternness Driver, or the Effective Differentiation Inhibition Concentration for a Differentiation Inhibitor, or the Effective Concentration for a TGF-beta Inhibitor.

“Effective TGF-beta Concentration” is the minimum concentration of a TGF-beta Inhibitor that creates at least about 15% greater colony diameter when combined with a Sternness Driver, measured at the end of the Stem Cell Proliferation Assay, compared to a Sternness Driver alone.

“Effective Release Rate” (mass/time) as used herein is the Effective Concentration (mass/volume)*30 uL/1 hour.

“Effective Sternness Driver Concentration” is the minimum concentration of a Sternness Driver that induces at least about 1.5-fold increase in number of supporting cells in a Stem Cell Proliferation Assay compared to the number of supporting cells in a Stem Cell Proliferation Assay performed without the Sternness Driver and with all other components present at the same concentrations.

“Eliminate” means to decrease to a level that is undetectable.

“Engraft” or “engraftment” refers to the process of stem or progenitor cell incorporation into a tissue of interest in vivo through contact with existing cells of the tissue. “Epithelial progenitor cell” refers to a multipotent cell which has the potential to become restricted to cell lineages resulting in epithelial cells.

“Epithelial stem cell” refers to a multipotent cell which has the potential to become committed to multiple cell lineages, including cell lineages resulting in epithelial cells.

“Fragment” refers to a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridize” refers to pairing to form a double-stranded molecule between complementary nucleotide bases (e.g., adenine (A) forms a base pair with thymine (T), as does guanine (G) with cytosine (C) in DNA) under suitable conditions of stringency. (See, e.g., Wahl, G. M. and S. L. Berger (1987) Methods Enzymol. 152:399; Kimmel, A. R. (1987) Methods Enzymol. 152:507).

An “inhibitor” refers to an agent that causes a decrease in the expression or activity of a target gene or protein, respectively. An “antagonist” can be an inhibitor, but is more specifically an agent that binds to a receptor, and which in turn decreases or eliminates binding by other molecules.

As used herein, an “inhibitory nucleic acid” is a double-stranded RNA, RNA interference, miRNA, siRNA, shRNA, or antisense RNA, or a portion thereof, or a mimetic thereof, that when administered to a mammalian cell results in a decrease in the expression of a target gene. Typically, a nucleic acid inhibitor comprises at least a portion of a target nucleic acid molecule, or an ortholog thereof, or comprises at least a portion of the complementary strand of a target nucleic acid molecule. Typically, expression of a target gene is reduced by 10%, 25%, 50%, 75%, or even 90-100%.

“In Vitro activity” refers to the level of expression or activity of an agent such as Lgr5, Sox2, and Sox9 in an in vitro population of cells. It may be measured, for example, via an “In Vitro Activity Assay” in cells derived from a reporter animal (e.g., a mouse). For example, for measuring Lgr5, Sox2, and Sox9 activity, respectively, cells from an Lgr5, Sox2, or Sox9 reporter animal may be dissociated to single cells, stained with propidium iodide (PI), and analyzed using a flow cytometer for expression of the reporter. Inner ear epithelial cells from wild-type (non-Lgr5, Sox2, or Sox9 reporter animals)that pass the same culturing and analyzing procedures can be used as a negative control. Typically, two population of cells are shown in the bivariate plot with one variable that includes at least one reporter, both reporter-positive and reporter-negative populations. Lgr5-positive cells are identified by gating GFP positive cell population. The percentage of Lgr5-positive cells are measured by gating GFP positive cell population against both GFP negative population and the negative control. The number of Lgr5-positive cells is calculated by multiplying the total number of cells by the percentage of Lgr5-positive cells. For cells derived from non-Lgr5-GFP mice, Lgr5 activity can be measured using an anti-Lgr5 antibody or quantitative-PCR on the Lgr5 gene. Sox2-positive cells are identified by gating GFP positive cell population. The percentage of Sox2-positive cells are measured by gating GFP positive cell population against both GFP negative population and the negative control. The number of Sox2-positive cells is calculated by multiplying the total number of cells by the percentage of Sox2-positive cells. For cells derived from non-Sox2-GFP mice, Sox2 activity can be measured using an anti-Sox2 antibody or quantitative-PCR on the Sox2 gene.

“In Vivo activity” as used herein is the level of expression or activity of an agent such as Lgr5, Sox9, or Sox2 in a subject. It may be measured, for example, via an “In Vivo Activity Assay” by removing an animal's inner ear and measuring Lgr5, Sox9, or Sox2 protein or Lgr5, Sox9, or Sox2 mRNA. Lgr5, Sox9, or Sox2 protein production can be measured using an anti-Lgr5 antibody, anti-Sox9 antibody, or an anti-Sox2 antibody, respectively, to measure fluorescence intensity as determined by imaging vestibular samples, where fluorescence intensity is used as a measure the target protein's presence. Western blots can be used with an anti-Lgr5 antibody, anti-Sox9 antibody, or an anti-Sox2 antibody where cells can be harvested from the treated organ to determine increases in Lgr5, Sox9, or Sox2 proteins, respectively. Quantitative-PCR or RNA in situ hybridization can be used to measure relative changes in Lgr5 Sox9, or Sox2 mRNA production, respectively, where cells can be harvested from the inner ear to determine changes in Lgr5, Sox9, or Sox2 mRNA. Alternatively, Lgr5, Sox9, or Sox2 expression can be measured using an Lgr5, Sox9, or Sox2 promoter driven GFP reporter transgenic system, where the presence or intensity GFP fluoresce can be directly detected using flow cytometry, imaging, or indirectly using an anti-GFP antibody.

“Increases” also means increases by at least 1-fold, for example, 1, 2, 3, 4, 5, 6, 7, 8, 9, 10, 15, 20, 30, 40, 50, 60, 70, 80, 90, 100, 200, 500, 1000-fold or more, for example, as compared to the level of a as compared to the level of a reference standard.

“Increasing” refers to increasing by at least 5%, for example, 5, 6, 7, 8, 9, 10, 15, 20, 25, 30, 35, 40, 45, 50, 55, 60, 65, 70, 75, 80, 85, 90, 95, 99, 100% or more, for example, as compared to the level of a reference.

“Intraauricular administration” refers to administration of a composition to the middle or inner ear of a subject by directly injecting the composition.

“Intravestibular” administration refers to direct injection of a composition across the tympanic membrane and across the round window membrane into the cochlea.

“Intravestibular” administration refers to direct injection of a composition across the tympanic membrane and across the round window membrane into the vestibular organs.

“Isolated” refers to a material that is free to varying degrees from components which normally accompany it as found in its native state. “Isolate” denotes a degree of separation from original source or surroundings.

“Lgr5” is an acronym for the Leucine-rich repeat-containing G-protein coupled receptor 5, also known as G-protein coupled receptor 49 (GPR49) or G-protein coupled receptor 67 (GPR67). It is a protein that in humans is encoded by the Lgr5 gene.

“Lgr5 activity” is defined as the level of activity of Lgr5 in a population of cells. In an in vitro cell population, Lgr5 activity may be measured in an In Vitro Activity Assay for Lgr5. In an in vivo cell population, Lgr5 activity may be measured in an In Vivo Activity Assay for Lgr5.

“Sox2” is an acronym for the SRY (sex determining region Y)-box 2, also known as ANOP2 and MCOPS3. It is a protein that in humans is encoded by the SOX2 gene.

“Sox2 activity” is defined as the level of activity of Sox2 in a population of cells. In an in vitro cell population, Sox2 activity may be measured in an In Vitro Activity Assay for Sox2. In an in vivo cell population, Sox2 activity may be measured in an In Vivo Activity Assay for Sox2.

“Sox9” is an acronym for the SRY (sex determining region Y)-box 9, also known as CMPD1, SRXY10, SRXX2, SRA1, and CMD1. It is a protein that in humans is encoded by the SOX9 gene.

“Sox9 activity” is defined as the level of activity of Sox9 in a population of cells. In an in vitro cell population, Sox9 activity may be measured in an In Vitro Activity Assay for Sox9. In an in vivo cell population, Sox9 activity may be measured in an In Vivo Activity assay for Sox9.

“Supporting cell” as used herein is an epithelial cell within the vestibular organs that is not a hair cell.

“Lgr5-positive cell” as used herein is a cell that expresses Lgr5. “Lgr5⁻ cell” as used herein is a cell that is not supporting. Lgr5-positive cells can be supporting cells.

“Sox2-positive cell” as used herein is a cell that expresses Sox2. “Sox2⁻ cell” as used herein is a cell that is not supporting. Sox2-positive cells can be supporting cells.

“Sox9-positive cell” as used herein is a cell that expresses Sox9. “Sox9⁻ cell” as used herein is a cell that is not supporting. Sox9-positive cells can be supporting cells.

“Lineage Tracing” as used herein is using a mouse line that enables fate tracing of any cell that expresses a target gene at the time of reporter induction. This can include hair cell or supporting cells genes (Sox2, Sox9, Lgr5, MyosinVIIa, Pou4f3, etc.). For example, lineage tracing may use an Lgr5-EGFP-IRES-creERT2 mouse crossed with a reporter mouse, which upon induction, allows one to trace the fate of cells that expressed Lgr5 at the time of induction. By further example, Lgr5 cells can be isolated into single cells and cultured in a Stem Cell Proliferation Assay to generate colonies, then subsequently differentiated in a Differentiation Assay and analyzed for cell fate by staining for hair cell and/or supporting cell proteins and determining the reporter colocalization with either hair cell or supporting cell staining to determine the Lgr5 cells' fate. In addition, lineage tracing can be performed in vestibular explants to track supporting cell or hair cell fate within the intact organ after treatment. For example, Lgr5 cell fate can be determined by isolating the vestibular organs from a Lgr5-EGFP-IRES-creERT2 mouse crossed with a reporter mouse, and inducing the reporter in Lgr5 cells before or during treatment. The organ can then be analyzed for cell fate by staining for hair cell and/or supporting cell proteins and determining the reporter colocalization with either hair cell or supporting cell staining to determine the Lgr5 cells' fate. In addition, lineage tracing can be performed in vivo track supporting cell or hair cell fate within the intact organ after treatment. For example, Lgr5 cell fate can be determined inducing a reporter in an Lgr5-EGFP-IRES-creERT2 mouse crossed with a reporter mouse, treating the animal, then isolating the vestibular organs. The organ can then be analyzed for cell fate by staining for hair cell and/or supporting cell proteins and determining the reporter colocalization with either hair cell or supporting cell staining to determine the Lgr5 cells' fate. Lineage tracing may be performed using alternative reporters of interest as is standard in the art.

“Mammal” refers to any mammal including but not limited to human, mouse, rat, sheep, monkey, goat, rabbit, hamster, horse, cow or pig.

“Mean Release Time” as used herein is the time in which one-half of an agent is released into phosphate buffered saline from a carrier in a Release Assay.

“Native Morphology” as used herein is means that tissue organization largely reflects the organization in a healthy tissue.

“Non-human mammal”, as used herein, refers to any mammal that is not a human.

As used in relevant context herein, the term “number” of cells can be 0, 1, or more cells.

“Vestibular Organs” as used herein refers to the utricle, saccule, and crista ampullaris of the semicircular canals.

“Organoid” or “epithelial organoid” refers to a cell cluster or aggregate that resembles an organ, or part of an organ, and possesses cell types relevant to that particular organ.

“Population” of cells refers to any number of cells greater than 1, but is preferably at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells.

“Progenitor cell” as used herein refers to a cell that, like a stem cell, has the tendency to differentiate into a specific type of cell, but is already more specific than a stem cell and is pushed to differentiate into its “target” cell.

In certain embodiments, the “purity” of any given compound in a composition may be specifically defined. For instance, certain compositions may comprise a compound that is at least 80, 85, 90, 91, 92, 93, 94, 95, 96, 97, 98, 99, or 100% pure, including all decimals in between, as measured, for example and by no means limiting, by high performance liquid chromatography (HPLC), a well-known form of column chromatography used frequently in biochemistry and analytical chemistry to separate, identify, and quantify compounds.

“Reference” means a standard or control condition (e.g., untreated with a test agent or combination of test agents).

“Release Assay” as used herein is a test in which the rate of release of an agent from a Biocompatible Matrix through dialysis membrane to a saline environment. An exemplary Release Assay may be performed by placing 30 microliters of a composition in 1 ml Phosphate Buffered Saline inside saline dialysis bag with a suitable cutoff, and placing the dialysis bag within 10 mL of Phosphate Buffered Saline at 37° C. The dialysis membrane size may be chosen based on agent size in order to allow the agent being assessed to exit the membrane. For small molecule release, a 3.5-5 kDa cutoff may be used. The agent may be a Stemness Driver, TGF-beta Inhibitor, or other agent. The Release Rate for a composition may change over time and may be measured in 1 hour increments.

“Representative Microscopy Sample” as used herein describes a sufficient number of fields of view within a cell culture system, a portion of extracted tissue, or an entire extracted organ that the average feature size or number being measured can reasonably be said to represent the average feature size or number if all relevant fields were measured. For example, in order to assess the hair cell counts at a frequency range on the Vestibular Organs, ImageJ software (NIH) can used to measure the total length of vestibular whole mounts and the length of individual counted segments. The total number of inner hair cells, outer hair cells, and supporting cells can be counted in the entire or fraction of any of the four vestibular segments of 1200-1400 μm (apical, mid-apical, mid-basal, and basal) at least 3 fields of view at 100 μm field size would be reasonably considered a Representative Microscopy Sample. A Representative Microscopy sample can include measurements within a field of view, which can be measured as cells per a given distance. A Representative Microscopy sample can be used to assess morphology, such as cell-cell contacts, vestibular architecture, and cellular components (e.g., bundles, synapses).

“Rosette Patterning” is a characteristic cell arrangement in the vestibular epithelia in which <5% hair cells are adjacent to other hair cells and are surrounded by supporting cells.

The term “sample” refers to a volume or mass obtained, provided, and/or subjected to analysis. In some embodiments, a sample is or comprises a tissue sample, cell sample, a fluid sample, and the like. In some embodiments, a sample is taken from (or is) a subject (e.g., a human or animal subject). In some embodiments, a tissue sample is or comprises brain, hair (including roots), buccal swabs, blood, saliva, semen, muscle, or from any internal organs, or cancer, precancerous, or tumor cells associated with any one of these. A fluid may be, but is not limited to, urine, blood, ascites, pleural fluid, spinal fluid, and the like. A body tissue can include, but is not limited to, brain, skin, muscle, endometrial, uterine, and cervical tissue or cancer, precancerous, or tumor cells associated with any one of these. In an embodiment, a body tissue is brain tissue or a brain tumor or cancer. Those of ordinary skill in the art will appreciate that, in some embodiments, a “sample” is a “primary sample” in that it is obtained from a source (e.g., a subject); in some embodiments, a “sample” is the result of processing of a primary sample, for example to remove certain potentially contaminating components and/or to isolate or purify certain components of interest “Self-renewal” refers to the process by which a stem cell divides to generate one (asymmetric division) or two (symmetric division) daughter cells with development potentials that are indistinguishable from those of the mother cell. Self-renewal involves both proliferation and the maintenance of an undifferentiated state.

“siRNA” refers to a double stranded RNA. Optimally, an siRNA is 18, 19, 20, 21, 22, 23 or 24 nucleotides in length and has a 2 base overhang at its 3′ end. These dsRNAs can be introduced to an individual cell or culture system. Such siRNAs are used to downregulate mRNA levels or promoter activity.

“Stem cell” refers to a multipotent cell having the capacity to self-renew and to differentiate into multiple cell lineages.

“Stem Cell Differentiation Assay” as used herein is an assay to determine the differentiation capacity of stem cells. In an exemplary Stem Cell Differentiation Assay, the number of cells for an initial cell population is harvested from a Atoh1-GFP mouse between the age of 3 to 7 days, by isolating the Vestibular Organs sensory epithelium, dissociating the epithelium into single cells, and passing the cells through a 40 um cell strainer. Approximately 5000 cells are entrapped in 40 μl of culture substrate (for example: Matrigel (Corning, Growth Factor Reduced)) and placed at the center of wells in a 24-well plate with 500 μl of an appropriate culture media, growth factors and agent being tested. Appropriate culture media and growth factors include Advanced DMEM/F12 with media Supplements (1×N2, 1×B27, 2 mM Glutamax, 10 mM HEPES, 1 mM N-acetylcysteine, and 100 U/ml Penicillin/100 μg/ml Streptomycin) and growth factors (50 ng/ml EGF, 50 ng/ml bFGF, and 50 ng/ml IGF-1) as well as the agent(s) being assessed are added into each well. Cells are cultured for 10 days in a standard cell culture incubator at 37° C. and 5% CO₂, with media change every 2 days. These cells are then cultured by removing the Stem Cell Proliferation Assay agents and replacing with Basal culture media and molecules to drive differentiation. An appropriate Basal culture media is Advanced DMEM/F12 supplemented with 1×N2, 1×B27, 2 mM Glutamax, 10 mM HEPES, 1 mM N-acetylcysteine, and 100 U/ml Penicillin/100 μg/ml Streptomycin and appropriate molecules to drive differentiation are 3 μM CHIR99021 and 5 μM DAPT for 10 days, with media change every 2 days. The number of hair cells in a population may be measured by using flow cytometry for GFP. Hair cell differentiation level can further be assessed using qPCR to measure hair cell marker (e.g., Myo7a) expression level normalized using suitable and unregulated references or housekeeping genes (e.g., Hprt). Hair cell differentiation level can also be assessed by immunostaining for hair cell markers (e.g., Myosin7a, vGlut3, Espin, PMCAs, Ribeye, conjugated-phalloidin, Atoh1, Pou4f3, etc.). Hair cell differentiation level can also be assessed by Western Blot for Myosin7a, vGlut3, Espin, PMCAs, Oncomodulin, Ribeye, Atoh1, Pou4f3.

“Stem Cell Assay” as used herein is an assay in which a cell or a cell population are tested for a series of criteria to determine whether the cell or cell population are stem cells or enriched in stem cells or stem cell markers. In a stem cell assay, the cell/cell population are tested for stem cell characteristics such as expression of Stem Cell Markers, and further optionally are tested for stem cell function, including the capacity of self-renewal and differentiation.

“Stem Cell Proliferator” as used herein is a compound that induces an increase in a population of cells which have the capacity for self-renewal and differentiation.

“Stem Cell Proliferation Assay” as used herein is an assay to determine the capacity for agent(s) to induce the creation of stem cells from a starting cell population. In an exemplary Stem Cell Proliferation Assay, the number of cells for an initial cell population is harvested from a Sox9-reporter mouse or a Sox2-GFP mouse such as a B6;129S-Sox2^{tm2Hoch}/(Jackson Lab Stock No: 017592) between the age of 0 to 7 days, by isolating the Vestibular Organs dissociating the organs into single cells. Approximately 5000 cells are entrapped in 40 μl of culture substrate (for example: Matrigel (Corning, Growth Factor Reduced)) and placed at the center of wells in a 24-well plate with 500 μl of an appropriate culture media, growth factors and agent being tested. Appropriate culture media and growth factors include Advanced DMEM/F12 with media Supplements (1×N2, 1×B27, 2 mM Glutamax, 10 mM HEPES, 1 mM N-acetylcysteine, and 100 U/ml Penicillin/100 μg/ml Streptomycin) and growth factors (50 ng/ml EGF, 50 ng/ml bFGF, and 50 ng/ml IGF-1) as well as the agent(s) being assessed are added into each well. Cells are cultured for 10 days in a standard cell culture incubator at 37° C. and 5% CO₂, with media change every 2 days. The number of supporting cells is quantified by counting the number of cells identified as supporting cells in a Sox9 or Sox2 In Vitro gene expression Assay (which includes PCR based methods and immunostaining). Additionally, Sox9 and/or Soc2 immunostaining is in some embodiments used to assess and quantify stem cell number. The fraction of cells that are supporting cells is quantified by dividing the number of cells identified as supporting cells in a cell population by the total number of cells present in the cell population. The average stem cell/supporting cell activity of a population is quantified by measuring the average mRNA expression level of Sox2 or Sox9 of the population normalized using suitable and unregulated references or housekeeping genes (e.g., Hprt). The number of hair cells in a population may be measured by staining with hair cell marker (e.g., MyosinVIIa), or using an endogenous reporter of hair cell genes (e.g., Pou4f3-GFP, Atoh1-nGFP) and analyzing using flow cytometry. The fraction of cells that are hair cells is quantified by dividing the number of cells identified as hair cells in a cell population by the total number of cells present in the cell population. Sox9, Sox2, and Lgr5 activity can be measured by qPCR.

“Stem Cell Markers” as used herein can be defined as gene products (e.g. protein, RNA, etc.) that specifically expressed in stem cells. One type of stem cell marker is gene products that are directly and specifically support the maintenance of stem cell identity. Examples include Lgr5 and Sox2, Sox9, and Bmi1. Additional stem cell markers can be identified using assays that were described in the literatures. To determine whether a gene is required for maintenance of stem cell identity, gain-of-function and loss-of-function studies can be used. In gain-of-function studies, over expression of specific gene product (the stem cell marker) would help maintain the stem cell identity. While in loss-of-function studies, removal of the stem cell marker would cause loss of the stem cell identity or induced the differentiation of stem cells. Another type of stem cell marker is gene that only expressed in stem cells but does not necessary to have specific function to maintain the identity of stem cells. This type of markers can be identified by comparing the gene expression signature of sorted stem cells and non-stem cells by assays such as micro-array and qPCR. This type of stem cell marker can be found in the literature. (e.g. Liu Q. et al., Int J Biochem Cell Biol. 2015 March; 60:99-111. http://www.ncbi.nlm.nih.gov/pubmed/25582750). Potential stem cell markers include Ccdc121, Gdf10, Opcm1, Phex, etc. The expression of stem cell markers such as Lgr5, Sox2, or Sox9 in a given cell or cell population can be measure using assays such as qPCR, immunohistochemistry, western blot, and RNA hybridization. The expression of stem cell markers can also be measured using transgenic cells express reporters which can indicate the expression of the given stem cell markers, e.g. Lgr5-GFP or Sox2-GFP, Sox9 reporter. Flow cytometry analysis can then be used to measure the activity of reporter expression. Fluorescence microscopy can also be used to directly visualize the expression of reporters. The expression of stem cell markers may further be determined using microarray analysis for global gene expression profile analysis. The gene expression profile of a given cell population or purified cell population can be compared with the gene expression profile of the stem cell to determine similarity between the 2 cell populations. Stem cell function can be measured by colony forming assay or sphere forming assay, self-renewal assay and differentiation assay. In colony (or sphere) forming assay, when cultured in appropriate culture media, the stem cell should be able to form colonies, on cell culture surface (e.g. cell culture dish) or embedded in cell culture substrate (e.g. Matrigel) or be able to form spheres when cultured in suspension. In colony/sphere forming assay, single stem cells are seeded at low cell density in appropriate culture media and allowed to proliferate for a given period of time (7-10 days). Colony formed are then counted and scored for stem cell marker expression as an indicator of stemness of the original cell. Optionally, the colonies that formed are then picked and passaged to test its self-renewal and differentiation potential. In self-renewal assay, when cultured in appropriate culture media, the cells should maintain stem cell marker (e.g. Lgr5) expression over at least one (e.g. 1, 2, 3, 4, 5, 10, 20, etc.) cell divisions. In a Stem Cell Differentiation Assay, when cultured in appropriate differentiation media, the cells should be able to generate hair cell which can be identified by hair cell marker expression measured by qPCR, immunostaining, western blot, RNA hybridization or flow cytometry.

“Stemness Driver” as used herein is a composition that induces proliferation of supporting cells, upregulates Lgr5 in cells, or maintains Lgr5 expression in cells, while maintaining the potential for self-renewal and the potential to differentiate into hair cells. Generally, stemness drivers upregulate at least one biomarker of post-natal stem cells. Stemness Drivers include but are not limited to Wnt agonist, GSK3-alpha inhibitor, or GSK3-beta inhibitors.

“Subject” includes humans and mammals (e.g., mice, rats, pigs, cats, dogs, and horses). In many embodiments, subjects are be mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

“Supporting Cell” as used herein in connection with a vestibular epithelium comprises epithelial cells within the Vestibular Organs that are not hair cells.

By “statistically significant”, it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Substantially” or “essentially” means nearly totally or completely, for instance, 95% or greater of some given quantity.

“Synergy” or “synergistic effect” is an effect which is greater than the sum of each of the effects taken separately; a greater than additive effect.

“TGF-β inhibitor” as used herein is a composition that reduces activity of TGF-β, including TGF-beta type I receptor inhibitors TGF-beta R1 kinase inhibitors, and inhibitors of any one or more of Alk4, Alk5, Alk7, Smad2, Smad3, Smad3, and Smad4.

“Tissue” is an ensemble of similar cells from the same origin that together carry out a specific function including, for example, tissue of vestibular, such as the Vestibular Organs.

“Transtympanic” administration refers to direct injection of a composition across the tympanic membrane into the middle ear.

“Treating” as used herein in connection with a cell population means delivering a substance to the population to effect an outcome. In the case of in vitro populations, the substance may be directly (or even indirectly) delivered to the population. In the case of in vivo populations, the substance may be delivered by administration to the host subject.

“Vestibular hair cell” as used herein includes Type I and/or Type II hair cells.

“Wnt activation” as used herein is an activation of the Wnt signaling pathway.

“Pharmaceutically-acceptable salt” includes both acid and base addition salts.

“Pharmaceutically-acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, l-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid,/toluenesulfonic acid, trifluoroacetic acid, undecylenic acid, and the like.

“Pharmaceutically-acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. For example, inorganic salts include, but are not limited to, ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Example organic bases used in certain embodiments include isopropylamine, diethylamine, ethanolamine, trimethylamine, dicyclohexylamine, choline and caffeine.

A description of exemplary embodiments of the disclosure follows.

The present disclosure relates to methods and compositions for activating the Wnt pathway and/or inhibiting TGF-beta activity.

In some aspects the present disclosure provides a method for controlled proliferation of stem cells by inducing stemness. In some embodiments, proliferation of stem cells is induced by contacting the stem cells with a Wnt agonist, GSK3-alpha inhibitor, or GSK3-beta inhibitor in combination with a TGF-beta inhibitor.

In some aspects the present disclosure relates to methods to prevent, reduce or treat the incidence and/or severity of disorders or diseases associated with absence or lack of certain tissue cells. In one aspect the present disclosure relates to methods to prevent, reduce or treat the incidence and/or severity of vestibular disorders involving vestibular hair cells, their progenitors, and optionally, and vestibulocochlear nerve. Of particular interest are those conditions that lead to permanent hearing loss where reduced number of hair cells may be responsible and/or decreased balance. Also of interest are those arising as an unwanted side-effect of ototoxic therapeutic drugs including cisplatin and its analogs, aminoglycoside antibiotics, salicylate and its analogs, or loop diuretics. In certain embodiments, the present disclosure relates to inducing, promoting, or enhancing the growth, proliferation or regeneration of vestibular tissue, particularly vestibular supporting cells and hair cells.

Among other things, the compositions and methods presented here are useful for the preparation of pharmaceutical formulations for the prophylaxis and/or treatment of acute and chronic ear disease, dizziness and balance problems, trauma during implantation of the inner ear prosthesis (insertion trauma), dizziness due to diseases of the inner ear area, dizziness related and/or as a symptom of Meniere's disease, vertigo related and/or as a symptom of Meniere's disease, benign paroxysmal positional vertigo (BPPV), labyrinthitis or vestibular neuritis, and ototoxicity.

When vestibular supporting cell populations are treated with a composition disclosed herein (e.g., a composition comprising (i) a Wnt agonist, a GSK3-alpha inhibitor, or a GSK3-beta inhibitor and (ii) a TGF-beta inhibitor), whether the population is in vivo or in vitro, the treated supporting cells exhibit stem-like behavior in that the treated supporting cells have the capacity to proliferate and differentiate and, more specifically, differentiate into vestibular hair cells. Preferably, the composition induces and maintains the supporting cells to produce daughter stem cells that can divide for many generations and maintain the ability to have a high proportion of the resulting cells differentiate into hair cells. In certain embodiments, the proliferating stem cells express stem cell markers which may include Lgr5, Sox2, Sox9, Opem1, Phex, 1in28, Lgr6, cyclin D1, Msx1, Myb, Kit, Gdnf3, Zic3, Dppa3, Dppa4, Dppa5, Nanog, Esrrb, Rex1, Dnmt3a, Dnmt3b, Dnmt31, Utf1, Tcl1, Oct4, Klf4, Pax6, Six2, Zic1, Zic2, Otx2, Bmi1, CDX2, STAT3, Smad1, Smad2, Smad2/3, Smad4, Smad5, and/or Smad7.

In some embodiments, the method of the present disclosure may be used to maintain, or even transiently increase sternness (i.e., self-renewal) of a pre-existing supporting cell population prior to significant hair cell formation. Morphological analyses with immunostaining (including cell counts) and lineage tracing across a Representative Microscopy Samples may be used to confirm expansion of one or more of these cell-types. In some embodiments, the pre-existing supporting cells comprise supporting cells. Morphological analyses with immunostaining (including cell counts) and qPCR and RNA hybridization may be used to confirm Lgr5 upregulation amongst the cell population.

Advantageously, the methods of the present disclosure achieve these goals without the use of genetic manipulation. Germ-line manipulation used in many academic studies is not a therapeutically desirable approach to treating hearing loss. In general, the therapy preferably involves the administration of a small molecule, peptide, antibody, or other non-nucleic acid molecule or nucleic acid delivery vector unaccompanied by gene therapy. In certain embodiments, the therapy involves the administration of a small organic molecule. Preferably, hearing protection or restoration is achieved through the use of a (non-genetic) therapeutic that is injected in the middle ear and diffuses into the vestibular organs.

The vestibular organs rely heavily on all present cell types, and the organization of these cells is important to their function. As supporting cells play an important role in neurotransmitter cycling and trophic support of hair cells. Thus, maintaining a rosette patterning within the Vestibular Organs may be important for function. Vestibular mechanics of the basement membrane activate hair cell transduction. Due to the high sensitivity of vestibular mechanics, it is also desirable to avoid masses of cells. In all, maintaining proper distribution and relation of hair cells and supporting cells along the basement membrane, even after proliferation, is likely a desired feature for hearing as supporting cell function and proper mechanics is necessary for normal hearing.

In one embodiment of the present disclosure, the cell density of hair cells in a vestibular cell population is expanded in a manner that maintains, or even establishes, the rosette pattern characteristic of vestibular epithelia.

In accordance with one aspect of the present disclosure, the cell density of hair cells may be increased in a population of vestibular cells comprising both hair cells and supporting cells. The vestibular cell population may be an in vivo population (i.e., comprised by the vestibular epithelium of a subject) or the vestibular cell population may be an in vitro (ex vivo) population. If the population is an in vitro population, the increase in cell density may be determined by reference to a Representative Microscopy Sample of the population taken prior and subsequent to any treatment. If the population is an in vivo population, the increase in cell density may be determined indirectly by determining an effect upon the hearing of the subject with an increase in hair cell density correlating to an improvement in hearing.

In one embodiment, supporting cells placed in a Stem Cell Proliferation Assay in the absence of neuronal cells form ribbon synapses.

In a native vestibular organs, patterning of hair cells and supporting cells occurs in a manner parallel to the basement membrane. In one embodiment of the present disclosure, the proliferation of supporting cells in a vestibular cell population is expanded in a manner parallel to the basement membrane

In one embodiment, the number of supporting cells in an initial vestibular cell population is selectively expanded by treating the initial vestibular cell population with a composition of the present disclosure (e.g., a composition containing an Effective Concentration of a Stemness Driver, e.g., a Wnt agonist, a GSK3-alpha inhibitor, or a GSK3-beta inhibitor, and, an Effective Concentration of a modulator of pathways that regulate cell cycle or plasticity, e.g., a TGF-beta inhibitor) to form an intermediate vestibular cell population and wherein the ratio of supporting cells to hair cells in the intermediate vestibular cell population exceeds the ratio of supporting cells to hair cells in the initial vestibular cell population. The expanded vestibular cell population may be, for example, an in vivo population, an in vitro population or even an in vitro explant. In one such embodiment, the ratio of supporting cells to hair cells in the intermediate vestibular cell population exceeds the ratio of supporting cells to hair cells in the initial vestibular cell population. For example, in one such embodiment the ratio of supporting cells to hair cells in the intermediate vestibular cell population exceeds the ratio of supporting cells to hair cells in the initial vestibular cell population by a factor of 1.1. By way of further example, in one such embodiment the ratio of supporting cells to hair cells in the intermediate vestibular cell population exceeds the ratio of supporting cells to hair cells in the initial vestibular cell population by a factor of 1.5. By way of further example, in one such embodiment the ratio of supporting cells to hair cells in the intermediate vestibular cell population exceeds the ratio of supporting cells to hair cells in the initial vestibular cell population by a factor of 2. By way of further example, in one such embodiment the ratio of supporting cells to hair cells in the intermediate vestibular cell population exceeds the ratio of supporting cells to hair cells in the initial vestibular cell population by a factor of 3. In each of the foregoing embodiments, the capacity of a composition of the present disclosure to expand a vestibular cell population as described in this paragraph may be determined by means of a Stem Cell Proliferation Assay.

In one embodiment, the number of stem cells in a vestibular cell population is expanded to form an intermediate vestibular cell population by treating a vestibular cell population with a composition of the present disclosure (e.g., a composition containing an Effective Concentration of a Stemness Driver, e.g., a Wnt agonist, a GSK3-alpha inhibitor, or a GSK3-beta inhibitor, and, an Effective Concentration of a modulator of pathways that regulate cell cycle or plasticity, e.g., a TGF-beta inhibitor), wherein the cell density of stem cells in the intermediate vestibular cell population exceeds the cell density of stem cells in the initial vestibular cell population. The treated vestibular cell population may be, for example, an in vivo population, an in vitro population or even an in vitro explant. In one such embodiment, the cell density of stem cells in the treated vestibular cell population exceeds the cell density of stem cells in the initial vestibular cell population by a factor of at least 1.1. For example, in one such embodiment the cell density of stem cells in the treated vestibular cell population exceeds the cell density of stem cells in the initial vestibular cell population by a factor of at least 1.25. For example, in one such embodiment the cell density of stem cells in the treated vestibular cell population exceeds the cell density of stem cells in the initial vestibular cell population by a factor of at least 1.5. By way of further example, in one such embodiment the cell density of stem cells in the treated vestibular cell population exceeds the cell density of stem cells in the initial vestibular cell population by a factor of at least 2. By way of further example, in one such embodiment the cell density of stem cells in the treated vestibular cell population exceeds the cell density of stem cells in the initial vestibular cell population by a factor of at least 3. In vitro vestibular cell populations may expand significantly more than in vivo populations; for example, in certain embodiments the cell density of stem cells in an expanded in vitro population of stem cells may be at least 4, 5, 6, 7, 8, 9, 10, 15, 20, 25, 30, 35, 40, 45, 50, 75, 100, 200, 300, 400, 500, 600, 700, 800, 900, 1000, 2000 or even 3000 times greater than the cell density of the stem cells in the initial vestibular cell population. In each of the foregoing embodiments, the capacity of a composition of the present disclosure to expand a vestibular cell population as described in this paragraph may be determined by means of a Stem Cell Proliferation Assay.

In accordance with one aspect of the present disclosure, a vestibular supporting cell population is treated with a with a composition of the present disclosure (e.g., a composition containing an Effective Concentration of a Stemness Driver, e.g., a Wnt agonist, a GSK3-alpha inhibitor, or a GSK3-beta inhibitor and an Effective Concentration of a modulator of pathways that regulate cell cycle or plasticity, e.g., a TGF-beta inhibitor) to increase the Sox9 activity of the population. For example, in one embodiment the composition has the capacity to increase and maintain the Sox9 activity of an in vitro population of vestibular supporting cells by factor of at least 1.2. By way of further example, in one such embodiment the composition has the capacity to increase the Sox9 activity of an in vitro population of vestibular supporting cells by factor of 1.5. By way of further example, in one such embodiment the composition has the capacity to increase the Sox9 activity of an in vitro population of vestibular supporting cells by factor of 2, 3, 5 10, 100, 500, 1000, 2000 or even 3000. Increases in Sox9 activity may also be observed for in vivo populations but the observed increase may be somewhat more modest. For example, in one embodiment the composition has the capacity to increase the Sox9 activity of an in vivo population of vestibular supporting cells by at least 5%. By way of further example, in one such embodiment the composition has the capacity to increase the Sox9 activity of an in vivo population of vestibular supporting cells by at least 10%. By way of further example, in one such embodiment the composition has the capacity to increase the Sox9 activity of an in vivo population of vestibular supporting cells by at least 20%. By way of further example, in one such embodiment the composition has the capacity to increase the Sox9 activity of an in vivo population of vestibular supporting cells by at least 30%. In each of the foregoing embodiments, the capacity of the composition for such an increase in Sox9 activity may be demonstrated, for example, in an In Vitro stem cell/supporting cell activity Assay and in an in vivo population may be demonstrated, for example, in an In Vivo stem cell/supporting cell activity Assay, as measured by isolating the organ and performing morphological analyses using immunostaining, endogenous fluorescent protein expression of Sox9, Sox2, and/or Lgr5, and qPCR for Sox9, Sox2, and/or Lgr5.

In accordance with one aspect of the present disclosure, a vestibular supporting cell population is treated with a with a composition of the present disclosure (e.g., a composition containing an Effective Concentration of a Stemness Driver, e.g., a Wnt agonist, a GSK3-alpha inhibitor, or a GSK3-beta inhibitor and an Effective Concentration of a modulator of pathways that regulate cell cycle or plasticity, e.g., a TGF-beta inhibitor) to increase the Sox2 activity of the population. For example, in one embodiment the composition has the capacity to increase and maintain the Sox2 activity of an in vitro population of vestibular supporting cells by factor of at least 1.2. By way of further example, in one such embodiment the composition has the capacity to increase the Sox2 activity of an in vitro population of vestibular supporting cells by factor of 1.5. By way of further example, in one such embodiment the composition has the capacity to increase the Sox2 activity of an in vitro population of vestibular supporting cells by factor of 2, 3, 5 10, 100, 500, 1000, 2000 or even 3000. Increases in Sox2 activity may also be observed for in vivo populations but the observed increase may be somewhat more modest. For example, in one embodiment the composition has the capacity to increase the Sox2 activity of an in vivo population of vestibular supporting cells by at least 5%. By way of further example, in one such embodiment the composition has the capacity to increase the Sox2 activity of an in vivo population of vestibular supporting cells by at least 10%. By way of further example, in one such embodiment the composition has the capacity to increase the Sox2 activity of an in vivo population of vestibular supporting cells by at least 20%. By way of further example, in one such embodiment the composition has the capacity to increase the Sox2 activity of an in vivo population of vestibular supporting cells by at least 30%. In each of the foregoing embodiments, the capacity of the composition for such an increase in Sox2 activity may be demonstrated, for example, in an In Vitro stem cell/supporting cell activity Assay and in an in vivo population may be demonstrated, for example, in an In Vivo stem cell/supporting cell activity Assay, as measured by isolating the organ and performing morphological analyses using immunostaining, endogenous fluorescent protein expression of Sox9, Sox2, and/or Lgr5, and qPCR for Sox9, Sox2, and/or Lgr5.

In accordance with one aspect of the present disclosure, a vestibular supporting cell population is treated with a with a composition of the present disclosure (e.g., a composition containing an Effective Concentration of a Stemness Driver, e.g., a Wnt agonist, a GSK3-alpha inhibitor, or a GSK3-beta inhibitor and an Effective Concentration of a modulator of pathways that regulate cell cycle or plasticity, e.g., a TGF-beta inhibitor) to increase the Lgr5 activity of the population. For example, in one embodiment the composition has the capacity to increase and maintain the Lgr5 activity of an in vitro population of vestibular supporting cells by factor of at least 1.2. By way of further example, in one such embodiment the composition has the capacity to increase the Lgr5 activity of an in vitro population of vestibular supporting cells by factor of 1.5. By way of further example, in one such embodiment the composition has the capacity to increase the Lgr5 activity of an in vitro population of vestibular supporting cells by factor of 2, 3, 5 10, 100, 500, 1000, 2000 or even 3000. Increases in Lgr5 activity may also be observed for in vivo populations but the observed increase may be somewhat more modest. For example, in one embodiment the composition has the capacity to increase the Lgr5 activity of an in vivo population of vestibular supporting cells by at least 5%. By way of further example, in one such embodiment the composition has the capacity to increase the Lgr5 activity of an in vivo population of vestibular supporting cells by at least 10%. By way of further example, in one such embodiment the composition has the capacity to increase the Lgr5 activity of an in vivo population of vestibular supporting cells by at least 20%. By way of further example, in one such embodiment the composition has the capacity to increase the Lgr5 activity of an in vivo population of vestibular supporting cells by at least 30%. In each of the foregoing embodiments, the capacity of the composition for such an increase in Lgr5 activity may be demonstrated, for example, in an In Vitro stem cell/supporting cell activity Assay and in an in vivo population may be demonstrated, for example, in an In Vivo stem cell/supporting cell activity Assay, as measured by isolating the organ and performing morphological analyses using immunostaining, endogenous fluorescent protein expression of Sox9, Sox2, and/or Lgr5, and qPCR for Sox9, Sox2, and/or Lgr5.

In addition to increasing the Sox9, Sox2, or Lgr5 activity of the population, the number of supporting cells in a vestibular cell population may be increased by treating a vestibular cell population containing supporting cells (whether in vivo or in vitro) with a compound of Formula I. In general, the cell density of the stem/progenitor supporting cells may expand relative to the initial cell population via one or more of several mechanisms. For example, in one such embodiment, newly generated supporting cells may be generated that have increased stem cell propensity (i.e., greater capacity to differentiate into hair cell). By way of further example, in one such embodiment no daughter supporting cells are generated by cell division, but pre-existing supporting cells are induced to differentiate into hair cells. By way of further example, in one such embodiment no daughter cells are generated by cell division, but supporting cells are activated to a greater level of Sox9 activity and the activated supporting cells are then able to differentiate into hair cells. Regardless of the mechanism, in one embodiment a composition of the present disclosure has the capacity to increase the cell density of supporting cells in an in vitro isolated cell population of vestibular supporting cells by factor of at least 5. By way of further example, in one such embodiment the compound has the capacity to increase the cell density of supporting cells in an in vitro population of vestibular supporting cells by factor of at least 10. By way of further example, in one such embodiment the compound has the capacity to increase the cell density of supporting cells in an in vitro population of vestibular supporting cells by factor of at least 100, at least 500, at least 1000 or even at least 2000. Increases in the cell density of supporting cells may also be observed for in vivo populations but the observed increase may be somewhat more modest. For example, in one embodiment the compound has the capacity to increase the cell density of supporting cells in an in vivo population of vestibular supporting cells by at least 5%. By way of further example, in one such embodiment the compound has the capacity to increase the cell density of supporting cells in an in vivo population of vestibular supporting cells by at least 10%. By way of further example, in one such embodiment the compound has the capacity to increase the cell density of supporting cells in an in vivo population of vestibular supporting cells by at least 20%. By way of further example, in one such embodiment the compound has the capacity to increase the cell density of supporting cells in an in vivo population of vestibular supporting cells by at least 30%. The capacity of the compound for such an increase in supporting cells in an in vitro population may be demonstrated, for example, in a Stem Cell Proliferation Assay or in an appropriate in vivo assay. In one embodiment, a compound of the present disclosure has the capacity to increase the number of supporting cells in the vestibular by inducing expression of Lgr5 in cells with absent or low detection levels of the protein, while maintaining Native Morphology. In one embodiment, a compound of the present disclosure has the capacity to increase the number of supporting cells in the vestibular by inducing expression of Sox9 in cells with absent or low detection levels of the protein, while maintaining Native Morphology and without producing Cell Aggregates.

In addition to increasing the cell density of supporting cells, in one embodiment the method of the present disclosure has the capacity to increase the ratio of supporting cells to hair cells in a vestibular cell population. In one embodiment, the number of supporting cells in an initial vestibular cell population is selectively expanded by treating the initial vestibular cell population with a compound of the present disclosure to form an expanded cell population and wherein the number of supporting cells in the expanded vestibular cell population at least equals the number of hair cells. The expanded vestibular cell population may be, for example, an in vivo population, an in vitro population or even an in vitro explant. In one such embodiment, the ratio of supporting cells to hair cells in the expanded vestibular cell population is at least 1:1. For example, in one such embodiment the ratio of supporting cells to hair cells in the expanded vestibular cell population is at least 1.5:1. By way of further example, in one such embodiment the ratio of supporting cells to hair cells in the expanded vestibular cell population is at least 2:1. By way of further example, in one such embodiment the ratio of supporting cells to hair cells in the expanded vestibular cell population is at least 3:1. By way of further example, in one such embodiment the ratio of supporting cells to hair cells in the expanded vestibular cell population is at least 4:1. By way of further example, in one such embodiment the ratio of supporting cells to hair cells in the expanded vestibular cell population is at least 5:1. In each of the foregoing embodiments, the capacity of a composition of the present disclosure to expand a vestibular cell population as described in this paragraph may be determined by means of a Stem Cell Proliferation Assay.

In certain embodiments, the method increases the fraction of the supporting cells to total cells on the sensory epithelium by at least 10%, 20%, 50%, 100%, 250% 500%, 1,000% or 5000%.

In certain embodiments, the method increases the supporting cells until they become at least 10, 20, 30, 50, 70, or 85% of the cells on the sensory epithelium, e.g. the Vestibular Organs.

In general, excessive proliferation of supporting cells in the vestibular organs is preferably avoided. In one embodiment, the method of the present disclosure has the capacity to expand a vestibular cell population without creating a protrusion of new cells beyond the native surface of the vestibular organs, e.g., a Cell Aggregate. In some embodiments, 30 days after placing a composition of the present disclosure (e.g., a composition containing an Effective Concentration of a Stemness Driver, e.g., a Wnt agonist, a GSK3-alpha inhibitor, or a GSK3-beta inhibitor and an Effective Concentration of a modulator of pathways that regulate cell cycle or plasticity, e.g., a TGF-beta inhibitor) on the round window membrane, the vestibular tissue has Native Morphology. In some embodiments, 30 days after placing a composition on the round window membrane, the vestibular tissue has Native Morphology and lacks Cell Aggregates. In some embodiments, 30 days after placing a composition on the round window membrane, the vestibular tissue has Native Morphology and at least 10, 20, 30, 50, 75, 90, 95, 98, or even at least 99% of the supporting cells in the Vestibular Organs are not part of Cell Aggregates.

In addition to expanding supporting cell populations, generally, and supporting cells, specifically, as described above, the method of the present disclosure has the capacity to maintain, in the daughter cells, the capacity to differentiate into hair cells. In in vivo populations, the maintenance of this capacity may be indirectly observed by an improvement in a subject's hearing. In in vitro populations, the maintenance of this capacity may be directly observed by an increase in the number of hair cells relative to a starting population or indirectly by measuring LGR5 activity, SOX2 activity, SOX9 activity or one or more of the other stem cell markers identified elsewhere herein.

In one embodiment, the capacity of the method to increase the sternness of a population of vestibular supporting cells, in general, or a population of supporting cells, in particular, may be correlated with an increase of Lgr5, Sox2, or Sox9 activity of an in vitro population of isolated supporting cells as determined by an In Vitro Activity Assay for Lgr5, Sox2, or Sox9. As previously noted, in one such embodiment, the compound has the capacity to increase the Lgr5, Sox2, or Sox9 activity of stem cells in the intermediate cell population by a factor of 5 on average relative to the respective Lgr5, Sox2, or Sox9 activity of the cells in the initial cell population. By way of further example, in one such embodiment the method has the capacity to increase the Lgr5, Sox2, or Sox9 activity of the stem cells genes in the intermediate cell population by a factor of 10 relative to the Lgr5, Sox2, or Sox9 activity of the cells in the initial cell population. By way of further example, in one such embodiment the method has the capacity to increase the Lgr5, Sox2, or Sox9 activity of the stem cells in the intermediate cell population by a factor of 100 relative to the Lgr5, Sox2, or Sox9 activity of the cells in the initial cell population. By way of further example, in one such embodiment the method has the capacity to increase the Lgr5, Sox2, or Sox9 activity of the stem cells in the intermediate cell population by a factor of 1000 relative to the Lgr5, Sox2, or Sox9 activity of the cells in the initial cell population. In each of the foregoing embodiments, the increase in the activity of stem cells in the cell population may be determined in vitro by immunostaining or endogenous fluorescent protein expression for target genes and analysis of their relative intensities via imaging analysis or flow cytometry, or using qPCR for target stem cell genes. The identity of the resulting stem cell population may optionally be further determined by stem cell assays including stem cell marker expression assay, colony forming assay, self-renewal assay and differentiation assay as defined in Stem cell assay.

In some embodiments, the method applied to an adult mammal produces a population of adult mammalian supporting cells that are in S-phase.

In one embodiment, after applying a Wnt agonist, a GSK3-alpha inhibitor, or GSK3-beta inhibitor and a modulator of pathways that regulate cell cycle or plasticity, (e.g., a TGF-beta inhibitor) to the round window of a mouse, the in vivo stem cell/supporting cell activity of a cell population in the Vestibular Organs increases 1.3×, 1.5×, up to 20× over baseline for a population that has not been exposed to the composition. In some embodiments, applying a composition to the round window of a mouse increases the average In vivo stem cell/supporting cell activity for cells in the Vestibular Organs is increased 1.3×, 1.5×, up to 20× over baseline for a population that has not been exposed to the composition.

In certain embodiments, the method increases the supporting cells until they become at least 10%, 7.5%, 10%, up to 100% of the supporting cell population by number.

In embodiments, the proliferation of the stem cells are be enhanced by adding a modulator of pathways that regulate cell cycle or plasticity, such as the TGF-beta pathways.

In some embodiments, a Sternness Driver (e.g., a Wnt agonist, a GSK3-alpha inhibitor, or a GSK3-beta inhibitor) may be used to drive the proliferation of Sox9⁺stem cells. In some cases, a Sternness Driver (e.g., a Wnt agonist, a GSK3-alpha inhibitor, or a GSK3-beta inhibitor) may also induce differentiation of Sox9⁺cells to hair cells. Examples of Sternness Drivers that may drive both proliferation and differentiation include Wnt agonist, a GSK3-alpha inhibitor, or a GSK3-beta inhibitors.

In some embodiments, a Sternness Driver (e.g., a Wnt agonist, a GSK3-alpha inhibitor, or a GSK3-beta inhibitor) may be used to drive the proliferation of Sox2⁺ stem cells. In some cases, a Sternness Driver (e.g., a Wnt agonist, a GSK3-alpha inhibitor, or GSK3-beta inhibitor) may also induce differentiation of Sox2⁺ cells to hair cells. Examples of Sternness Drivers that may drive both proliferation and differentiation include Wnt agonist, GSK3-alpha inhibitor, or GSK3-beta inhibitor.

In some embodiments, a Sternness Driver (e.g., a Wnt agonist, a GSK3-alpha inhibitor, or a GSK3-beta inhibitor) may be used to drive the proliferation of Lgr5⁺ stem cells. In some cases, a Sternness Driver (e.g., a Wnt agonist, a GSK3-alpha inhibitor, or a GSK3-beta inhibitor) may also induce differentiation of Lgr5⁺ cells to hair cells. Examples of Sternness Drivers that may drive both proliferation and differentiation include Wnt agonist, GSK3-alpha inhibitor, or GSK3-beta inhibitors.

In certain embodiments, a composition has the capacity to increase the percentage of supporting cell in a vestibular organ by 5%, 10%, 25%, 50%, or 80%. In certain embodiments, a combination of (i) a Wnt agonist, GSK3-alpha inhibitor, or GSK3-beta inhibitor, and (ii) a TGF-beta inhibitor is used, which has the capacity to increase the percentage of Sox9⁺cells in a vestibular organ by 5%, 10%, 25%, 50%, or 80%.

### Sternness Drivers

Classes of Wnt agonist for use in various embodiments of the compositions and methods disclosed herein include but are not limited to those listed in Column A of Table 1. Specific Wnt agonist for use in various embodiments of the compositions and methods disclosed herein include but are not limited to those listed in Column B of Table 1. All agents listed in Table 1 column B are understood to include derivatives or pharmaceutically-acceptable salts thereof. All classes listed in Table 1 column A are understood to include both agents comprising that class and derivatives or pharmaceutically-acceptable salts thereof.

Classes of GSK3-alpha inhibitor for use in various embodiments of the compositions and methods disclosed herein include but are not limited to those listed in Column A of Table 6. Specific GSK3-alpha inhibitor for use in various embodiments of the compositions and methods disclosed herein include but are not limited to those listed in Column B of Table 6. All agents listed in Table 6 column B are understood to include derivatives or pharmaceutically-acceptable salts thereof. All classes listed in Table 6 column A are understood to include both agents comprising that class and derivatives or pharmaceutically-acceptable salts thereof.

Classes of GSK3-beta inhibitor for use in various embodiments of the compositions and methods disclosed herein include but are not limited to those listed in Column A of Table 2. Specific GSK3-beta inhibitor for use in various embodiments of the compositions and methods disclosed herein include but are not limited to those listed in Column B of Table 2. All agents listed in Table 2 column B are understood to include derivatives or pharmaceutically-acceptable salts thereof. All classes listed in Table 2 column A are understood to include both agents comprising that class and derivatives or pharmaceutically-acceptable salts thereof.

Exemplary Wnt agonists within the present disclosure appear in Table 1.

TABLE 1
Wnt agonists

| Column A | Column B | CAS Number |
|---|---|---|
| Wnt Ligand | Wnt-1 | Protein |
| | Wnt-2/Irp | Protein |
| | (Int-I-related protein) | |
| | Wnt-2b/13 | Protein |
| | Wnt-3/Int-4 | Protein |
| | Wnt-3a | Protein |
| | Wnt-4 | Protein |
| | Wnt-5a | Protein |
| | Wnt-5b | Protein |
| | Wnt-6 | Protein |
| | Wnt-7a | Protein |
| | Wnt-7b | Protein |
| | Wnt-8a/8d | Protein |
| | Wnt-8b | Protein |
| | Wnt-9a/14 | Protein |
| | Wnt-9b/14b/15 | Protein |
| | Wnt-10a | Protein |
| | Wnt-10b/12 | Protein |
| | Wnt-11 | Protein |
| | Wnt-16 | Protein |
| Wnt Related Protein | R-Spondin 1/2/3/4 | |
| | Norrin | |
| Target | Agent | CAS Number |
| Wnt-3a/Dkk-1 | Compound 1 | 1084833-94-2 |
| Wnt-3a/Dkk-1 | Compound 25 | 1084834-05-8 |
| | BML-284 | 853220-52-7 |
| PP2A | IQ 1 | 331001-62-8 |
| beta-catenin | DCA | 56-47-3 |
| ARFGAP1 | QS 11 | 944328-88-5 |
| | WASP-1, | 352328-82-6 |
| | ZINC00087877 | |
| sFRP-1 inhibitor | WAY 316606 | 915759-45-4 |
| DKK1 inhibitor | WAY-262611 | 1123231-07-1 |
| Axin | HLY78 | 854847-61-3 |
| Axin | SKL2001 | 909089-13-0 |
| | Cpd1 | 1357473-75-6 |
| | Cpd2 | 1228659-47-9 |
| van-Gogh-like receptor proteins | Compound 109 | 1314885-81-8 |
| (Vangl) | | |
| Disrupts the Axin Complex | ISX 9 | 832115-62-5 |
| | Cmpd 71 | 1622429-71-3 |
| | Cmpd 2 | 1360540-82-4 |
| MEK | Selumetinib | 606143-52-6 |
| | (AZD6244) | |
| | Radicicol | 12772-57-5 |

Wnt-agonists also include but are not limited to those agents that increase Wnt activity by more than 5, 10, 20, 30, or 50% when an otic cell line or primary cells obtained from otic tissue is exposed to the agonist at pharmaceutically-acceptable concentrations and activity is assessed via Western blotting or other standard methods in the literature. In certain embodiments, the composition comprises a Wnt agonist, GSK3-alpha inhibitor, or GSK3-beta inhibitor increasing Wnt activity by more than 5, 10, 20, 30, or 50% using conditions described in this paragraph in combination with a TGF-beta inhibitor. “Highly potent Wnt agonist, GSK3-alpha inhibitor, or GSK3-beta inhibitor are those that increase Wnt activity by more than 50% when an otic cell line or primary cells obtained from otic tissue is exposed to the agonist at pharmaceutically-acceptable concentrations and activity is assessed via Western blotting or other standard methods in the literature.

Exemplary GSK3-beta inhibitors within the present disclosure appear in Table 2.

TABLE 2
GSK3β Inhibitors

| Column A | Column B | |
|---|---|---|
| Class | Agent | CAS Number |
| Acid | Valproic Acid, Sodium Salt | 99-66-1 |
| Acid | Bikinin | 188011-69-0 |
| Pyrroloazepine | Hymenialdisine | 82005-12-7 |
| Aloisines | Aloisine A | 496864-16-5 |
| Aloisines | Aloisine B | 496864-14-3 |
| Aloisines | TWS119 | 1507095-58-0 |
| Aminopyrimidine | CT20026 | 403808-63-9 |
| Aminopyrimidine | CHIR99021 (CT99021) | 252917-06-9 |
| Aminopyrimidine | CHIR98014 (CT98014) | 252935-94-7 |
| Aminopyrimidine | CHIR98023 (CT98023) | 252904-84-0 |
| Aminopyrimidine | CHIR98024 (CT98024) | 556813-39-9 |
| Aminopyrimidinyl | GSK-3β Inhibitor XVIII | 1139875-74-3 |
| Aminopyrimidinyl | CGP60474 | 164658-13-3 |
| Miscellaneous | AZD2858 (AR28) | 486424-20-8 |
| Miscellaneous | CID 755673 | 521937-07-5 |
| Miscellaneous | TCS 2002 | 1005201-24-0 |
| Miscellaneous | Dibromocantharelline | 101481-34-9 |
| Dihydropyridine | ML320 | 1597438-84-0 |
| Flavone | Flavopiridol | 146426-40-6 |
| Furopyrimidine | Compound 100 | 744255-19-4 |
| Hymenidin | Hymenidin | 107019-95-4 |
| Indirubins | 6-Bromoindirubin-3-acetoxime | 667463-85-6 |
| Indirubins | GSK-3 Inhibitor IX | 667463-62-9 |
| Indirubins | Indirubin-3′-monoxime | 160807-49-8 |
| Indirubins | 5-Iodo-indirubin-3′-monoxime | 331467-03-9 |
| Indirubins | Indirubin-5-sulfonic acid sodium | 331467-05-1 |
| | salt | |
| Indirubins | Indirubin | 479-41-4 |
| Indirubins | GSK-3 Inhibitor X | 740841-15-0 |
| Inorganic atom | Lithium Chloride | |
| Inorganic atom | Beryllium | |
| Inorganic atom | Zinc | |
| Inorganic atom | Tungstate | |
| Isonicotinamides | Compound 39 | 1772824-10-8 |
| Isonicotinamides | Compound 29 | 1772823-37-6 |
| Isonicotinamides | Compound 33 | 1772823-64-9 |
| Azaindolylmaleimide | Compound 29 | 436866-61-4 |
| Azaindolylmaleimide | Compound 46 | 682807-74-5 |
| Bisindolylmaleimide | Compound 5a | 436866-54-5 |
| Bisindolylmaleimide | GF109203x | 176504-36-2 |
| Bisindolylmaleimide | Ro318220 | 125314-64-9 |
| Bisindolylmaleimide | Bisindolylmaleimide X HCl | 131848-97-0 |
| Bisindolylmaleimide | Enzastaurin (LY317615) | 170364-57-5 |
| Maleimide | I5 | 264217-24-5 |
| Maleimide | SB-216763 | 280744-09-4 |
| Maleimide | SB-415286 (SB-41528) | 264218-23-7 |
| Maleimide | 3F8 | 159109-11-2 |
| Maleimide | TCS 21311 | 1260181-14-3 |
| Maleimide | GSK-3 inhibitor 1 | 603272-51-1 |
| Maleimide | LY2090314 | 603288-22-8 |
| Maleimide | 603281-31-8 | 603281-31-8 |
| Maleimide | IM-12 | 1129669-05-1 |
| Maleimide | Compound 34 | 396091-16-0 |
| Maleimide | KT 5720 | 108068-98-0 |
| Maleimide | Isogranulatimide | 244148-46-7 |
| Maleimide | GSK-3β Inhibitor XI | 626604-39-5 |
| Maleimide | BIP-135 | 941575-71-9 |
| Maleimide | CP21R7 | 125314-13-8 |
| Maleimide | Tivantinib | 905854-02-6 |
| Organometallic | Compound lambda-OS1 | 1291104-51-2, 1292843-11-8 |
| Organometallic | HB12 | 800384-87-6 |
| Organometallic | DW12 | 861251-33-4 |
| Organometallic | NP309 | 937810-13-4 |
| Organometallic | (RRu)-HB1229 | |
| Organometallic | (RRu)-NP549 | |
| Organometallic | Compound 3 | 1498285-39-4, 1498285-48-5 |
| Organometallic | Compound (R)-DW12 | 1047684-07-0 |
| Isoindolone | Staurosporine | 62996-74-1 |
| Pyrazolone | GSK-3beta Inhibitor XXVI | 871843-09-3 |
| Manzamines | Manzamine A | 104196-68-1 |
| Oxadiazol | TC-G 24 | 1257256-44-2 |
| Oxadiazol | Compound 14d | 1374671-64-3 |
| Oxadiazol | Compound 15b | 1374671-66-5 |
| Oxadiazol | Compound 20x | 1005201-80-8 |
| Oxadiazol | GSK-3 Inhibitor II | 478482-75-6 |
| Oxadiazol | GSK3 Inhibitor, 2 | 1377154-01-2 |
| Oxindole | SU9516 | 77090-84-1 |
| Oxindole | AZD1080 | 612487-72-6 |
| Paullone | Kenpaullone | 142273-20-9 |
| Paullone | Cmpd 17b | 408532-42-3 |
| Paullones | Azakenpaullone | 676596-65-9 |
| Paullones | Alsterpaullone | 237430-03-4 |
| Paullones | Alsterpaullone CN Ethyl | 852529-97-0 |
| Paullones | Cazpaullone | 914088-64-5 |
| Peptide | FRATtide | |
| Peptide | L803 | |
| Peptides | L803-mts | |
| Pyrazole | GSK-3 Inhibitor XXII | 1195901-31-5 |
| Pyrazole | Compound 4a | 1627557-91-8 |
| Pyrazole | Compound 4t | 1627558-10-4 |
| Pyrazole | Compound 4z | 1627558-16-0 |
| Pyrazole | AT 7519 | 844442-38-2 |
| Pyrazolopyridine | Pyrazolopyridine 9 | 923029-74-7 |
| Pyrazolopyridine | Pyrazolopyridine 18 | 405221-39-8 |
| Pyrazolopyridine | Pyrazolopyridine 34 | 583039-27-4 |
| Pyrazolopyridines | Compound 14 | 583038-63-5 |
| Pyrazolopyridines | Compound 23 | 583038-76-0 |
| Pyrazolopyridines | Compound 14 | 583038-63-5 |
| Pyrazolopyridazines | Compound 18 | 405223-20-3 |
| Pyrazolopyridazines | Compound 19 | 405223-71-4 |
| Pyrazoloquinoxaline | NSC 693868 (Compound 1) | 40254-90-8 |
| Pyridinone | Compound 150 | 1282042-18-5 |
| Quinazolin | GSK-3 Inhibitor XIII | 404828-08-6 |
| Quinolinecarb | VP0.7 | 331963-23-6 |
| Quinolinecarboxamide | | 1132813-46-7 |
| Quinolinecarboxamide | | 1132812-98-6 |
| Quinolinecarboxamide | | 950727-66-9 |
| Pyrazoloquinoxaline | NSC 693868 (Compound 1) | 40254-90-8 |
| Halomethylketones | Compound 17 | 62673-69-2 |
| Halomethylketones | GSK-3β Inhibitor VII | 99-73-0 |
| Halomethylketones | GSK-3β Inhibitor VI | 62673-69-2 |
| Furanosesquiterpenes | Palinurin | 254901-27-4 |
| Furanosesquiterpenes | Tricantin | 853885-55-9 |
| Thiadiazolidindiones | GSK-3β Inhibitor I | 327036-89-5 |
| Thiadiazolidindiones | NP031115 | 1400575-57-6 |
| Thiadiazolidindiones | NP031112 (Tideglusib) | 865854-05-3 |
| Triazolpyrimidine | Compound 90 | 91322-11-1 |
| Triazolpyrimidine | Compound 92 | 1043429-30-6 |
| Urea | GSK-3β Inh. VIII AR-A014418 | 487021-52-3 |
| Urea | A-1070722 | 1384424-80-9 |
| Pyrrolopyridinyl | Compound 27 | 2025388-25-2 |
| Pyrrolopyridinyl | Compound 12 | 2025388-10-5 |
| Publication | NP-103 | No Structure |
| Publication | CG-301338 | No Structure |
| Publication | SAR 502250 | No Structure |
| Publication | XD-4241 | No Structure |
| Publication | CEP-16805 | No Structure |
| Publication | AZ13282107 | No Structure |
| Publication | SAR 502250 (Sanofi) | 1073653-58-3 |
| Publication | AR79 | |
| Publication | AZ13282107 | |
| Patent | GI179186X | |
| Patent | CT118637 | |
| Patent | CP-70949 | |
| Patent | GW784752X | |
| Patent | GW784775X | |
| Publication | CT73911 | |
| Publication | LY2064827 | |
| Publication | 705701 | |
| Publication | 708244 | |
| Publication | 709125 | |
| Patent | WO 2008077138 A1 | |
| Patent | WO 2003037891 A1 | |
| Patent | U.S. Pat. No. 8,207,216 B2 | |
| Patent | U.S. Pat. No. 8,071,591 B2 | |
| Patent | CN 1319968 C | |
| Patent | U.S. Pat. No. 7,514,445 B2 | |
| Patent | CN 101341138 B | |
| Patent | EP 1961748 A2 | |
| Patent | WO 2010104205 A1 | |
| Patent | US 20100292205 A1 | |
| Patent | WO 2014003098 A1 | |
| Patent | WO 2011089416 A1 | |
| Patent | EP 1739087 A1 | |
| Patent | WO 2001085685 A1 | |
| Patent | US 20070088080 A1 | |
| Patent | WO 2006018633 A1 | |
| Patent | WO 2009017453 A1 | |
| Patent | WO 2014050779 A1 | |
| Patent | WO2006100490A1/EP 1863904 | |
| | A1 | |
| Patent | WO 2014013255 A1 | |
| Patent | WO2009017455 A1 | |
| Patent | EP 2765188 A1 | |
| Patent | WO 2014083132 A1 | |
| Patent | U.S. Pat. No. 8,771,754 B2 | |
| Patent | WO 2013124413 A1 | |
| Patent | WO 2014059383 A1 | |
| Patent | WO 2010075551 A1 | |
| Patent | U.S. Pat. No. 8,686,042 B2 | |
| Patent | WO 2007102770 A1 | |

J. Med. Chem. 2016, 59, 9018-9034

Exemplary Notch agonists within the present disclosure appear in Table 3.

TABLE 3
Notch Agonist

| | | CAS |
|---|---|---|
| Column A | Column B | Number |
| Natural receptor Ligands | Jagged 1 | Protein |
| | Jagged 2 | Protein |
| | Delta-like 1 | Protein |
| | Delta-like 2 | Protein |
| | Delta-like 3 | Protein |
| | Delta-like 4 | Protein |
| | DSL peptide | Protein |
| | Delta 1 | Protein |
| | Delta D | Protein |
| Receptor antibodies | Notch 1 antibody | Protein |
| Inhibition of Suppressor of | | |
| Deltex-mediated receptor | | |
| ubiquitination/ | | |
| degradation | | |
| Downregulation of | Notchless | Protein |
| negative modulators | | |
| of Notch activity | | |
| | Numb | Protein |
| Portion of Jag-1 residue | CDDYYYGFGCNKFCRPR | Peptide |
| 188-204 | | |

Classes of Notch agonists for use in various embodiments of the compositions and methods disclosed herein include but are not limited to those listed in Column A of Table 3. Specific Notch agonists for use in various embodiments of the compositions and methods disclosed herein include but are not limited to those listed in Column B of Table 3. All agents listed in Table 3 column B are understood to include derivatives or pharmaceutically-acceptable salts thereof. All classes listed in Table 3 column A are understood to include both agents comprising that class and derivatives or pharmaceutically-acceptable salts thereof.

Exemplary HDAC Inhibitors within the present disclosure appear in Table 4.

TABLE 4
HDAC Inhibitors

| Column A | Column B | |
|---|---|---|
| Class | Agent | CAS Number |
| Aliphatic Acid | Valproic Acid | 99-66-1 |
| Aliphatic Acid | Phenyl butyrate | 1821-12-1 |
| Aliphatic Acid | Butyrate | 107-92-6 |
| Aliphatic Acid | 2-hexyl-4-pentynoic acid | 96017-59-3 |
| Aliphatic Acid | S-2-hexyl-4-pentynoic acid | 185463-37-0 |
| Aliphatic Acid | R-2-hexyl-4-pentynoic acid | 185463-38-1 |
| Aliphatic Acid | 2-pentyl-4-pentynoic acid | 176638-49-6 |
| Aliphatic Acid | R-2-pentyl-4-pentynoic acid | 675831-45-5 |
| Aliphatic Acid | S-2-pentyl-4-pentynoic acid | 675831-46-6 |
| Aliphatic Acid | 2-propylpent-4-ynoic acid | 24102-11-2 |
| Aliphatic Acid | 2-ethyl-4-Pentynoic acid | 245079-04-3 |
| Aliphatic Acid | 3-propyl-heptanoic acid | 96185-13-6 |
| Aliphatic Acid | 2,2,3,3-Tetramethylcyclopropanecarboxylic | 15641-58-4 |
| | acid | |
| Aliphatic Acid | 1-Methyl-1- | 1123-25-7 |
| | cyclohexanecarboxylic acid | |
| Aliphatic Acid | 4-oxo-6-[4-(1-piperidinyl)phenyl]- | 1632052-48-2 |
| | (5E)-5-Hexenoic acid, | |
| Aliphatic Acid | 3-[4-(4-phenyl-1-piperazinyl)phenyl]- | 1632052-55-1 |
| | (2E)-2-Propenoic acid | |
| Aliphatic Acid | 4-oxo-6-[4-(4-phenyl-1- | 1632052-51-7 |
| | piperazinyl)phenyl]-(5E)-5- | |
| | Hexenoic acid | |
| Aliphatic Acid Ester | AN-9 | 122110-53-6 |
| Amine | 932718-22-4 | 932718-22-4 |
| Benzamide | Entinostat (MS-275) | 209783-80-2 |
| Benzamide | Mocetinostat (MGCD0103) | 726169-73-9 |
| Benzamide | Tacedinaline | 112522-64-2 |
| Benzamide | BML-210 | 537034-17-6 |
| Benzamide | NKL 22 | 537034-15-4 |
| Benzamide | RGFP109 | 1215493-56-3 |
| Benzamide | RGFP136 | 1215493-97-2 |
| Benzamide | RGFP966 | 1357389-11-7 |
| Benzamide | 4SC-202 | 1186222-89-8 |
| Benzamide | HDAC Inhibitor IV | 537034-15-4 |
| Benzamide | Chidamide | 743438-44-0 |
| Benzamide | TC-H 106, HDAC Inhibitor VII | 937039-45-7 |
| Cyclic peptide | Romidepsin | 128517-07-7 |
| Cyclic peptide | Trapoxin A | 133155-89-2 |
| Cyclic peptide | HC Toxin | 83209-65-8 |
| Cyclic peptide | Apicidin | 183506-66-3 |
| Cyclic Peptide | Thailandepsin A | 1269219-30-8 |
| Cyclic peptide | Dihydrochlamydocin | 52574-64-8 |
| Epoxide | (−)-Depudecin | 139508-73-9 |
| Epoxide | Parthenolide | 20554-84-1 |
| Hydroxamate | Trichostatin A (TSA) | |
| Hydroxamate | Trichostatin A (TSA) | 58880-19-6 |
| Hydroxamate | SAHA (Zolinza, vorinostat) | 149647-78-9 |
| Hydroxamate | 4-iodo-SAHA | 1219807-87-0 |
| Hydroxamate | SBHA | 38937-66-5 |
| Hydroxamate | CBHA | 174664-65-4 |
| Hydroxamate | LAQ-824 | 591207-53-3 |
| Hydroxamate | PDX-101 (belinostat) | 866323-14-0 |
| Hydroxamate | LBH-589 (panobinostat) | 404950-80-7 |
| Hydroxamate | ITF2357 (Givinostat) | 497833-27-9 |
| Hydroxamate | PCI-34051 | 950762-95-5 |
| Hydroxamate | PCI-24781 (Abexinostat) | 783355-60-2 |
| Hydroxamate | Tubastatin A | 1252003-15-8 |
| Hydroxamate | CUDC-101 | 1012054-59-9 |
| Hydroxamate | Oxamflatin | 151720-43-3 |
| Hydroxamate | ITF2357 | 497833-27-9 |
| Hydroxamate | Bufexamac | 2438-72-4 |
| Hydroxamate | APHA Compound 8 | 676599-90-9 |
| Hydroxamate | HDAC Inhibitor XXIV | 854779-95-6 |
| Hydroxamate | Tubacin | 537049-40-4 |
| Hydroxamate | Butyrylhydroxamic acid | 4312-91-8 |
| Hydroxamate | MC 1568 | 852475-26-4 |
| Hydroxamate | SB939 (Pracinostat) | 929016-96-6 |
| Hydroxamate | 4SC-201 (Resminostat) | 864814-88-0 |
| Hydroxamate | Tefinostat (CHR-2845) | 914382-60-8 |
| Hydroxamate | CHR-3996 | 1256448-47-1 |
| Hydroxamate | NSC 57457 | 6953-61-3 |
| Hydroxamate | CG200745 | 936221-33-9 |
| Hydroxamate | ACY1215 | 1316214-52-4 |
| Hydroxamate | Nexturastat A | 1403783-31-2 |
| Hydroxamate | Droxinostat | 99873-43-5 |
| Hydroxamate | Scriptaid | 287383-59-9 |
| Hydroxamate | BRD9757 | 1423058-85-8 |
| Hydroxamate | HPOB | 1429651-50-2 |
| Hydroxamate | CAY10603 | 1045792-66-2 |
| Hydroxamate | HDAC6 Inhibitor III | 1450618-49-1 |
| Hydroxamate | M 344 | 251456-60-7 |
| Hydroxamate | 4-(dimethylamino)-N-[6- | 193551-00-7 |
| | (hydroxyamino)-6-oxohexyl]- | |
| | benzamide | |
| Hydroxamate | (S)-HDAC-42 | 935881-37-1 |
| Hydroxamate | HNHA | 926908-04-5 |
| Hydroxamate | Pyroxamide | 382180-17-8 |
| Hydroxamate | HDAC Inhibitor VI | 926908-04-5 |
| Hydroxamate | HDAC Inhibitor II | 174664-65-4 |
| Hydroxamate | LMK235 | 1418033-25-6 |
| Hydroxamate | HDAC-IN-1 | 1239610-44-6 |
| Hydroxamate | VAHA | 106132-78-9 |
| Ketone-CF3 | Compound 6e | 946500-31-8 |
| Ketone-CF3 | Compound 6H | 946500-39-6 |
| Ketone-CF3 | Compound 27 | 946499-86-1 |
| Ketone | Compound 43 | 891259-76-0 |
| Ketone-a-ketoamides | 436150-82-2 | 436150-82-2 |
| Polyketide | Ratjadone A | 163564-92-9 |
| Silylalcohol | 1587636-32-5 | 1587636-32-5 |
| Sulphonyl Urea | 960130-17-0 | 960130-17-0 |
| Sulphonamide | 1587636-33-6 | 1587636-33-6 |
| Sulphonamide | 329967-25-1 | 329967-25-1 |
| Thiol | 1428536-05-3 | 1428536-05-3 |
| Thiol | 908860-21-9 | 908860-21-9 |
| Thiol | 828920-13-4 | 828920-13-4 |
| Thiol | 1368806-68-1 | 1368806-68-1 |
| Thiol | 827036-76-0 | 827036-76-0 |
| Thioester | TCS HDAC6 20b | 956154-63-5 |
| Thioester | PTACH | 848354-66-5 |
| Thioester | KD 5170 | 940943-37-3 |
| Thioester | HDAC Inhibitor XXII | 848354-66-5 |
| Thioketone | SIRT1/2 Inhibitor VII | 143034-06-4 |
| Tropones | 46189-88-2 | 46189-88-2 |
| Tropones | 1411673-95-4 | 1411673-95-4 |
| Non classical | TMP269 | 1314890-29-3 |
| Non classical | Tasquinimod | 254964-60-8 |

Classes of HDAC inhibitors for use in various embodiments of the compositions and methods disclosed herein include but are not limited to those listed in Column A of Table 4. Specific HDAC inhibitors for use in various embodiments of the compositions and methods disclosed herein include but are not limited to those listed in Column B of Table 4. All agents listed in Table 4 column B are understood to include derivatives or pharmaceutically-acceptable salts thereof. All classes listed in Table 4, Column A are understood to include both agents comprising that class and derivatives or pharmaceutically-acceptable salts thereof.

In certain embodiments, the one or more additional agents comprises a TGF-beta type I receptor inhibitor. Exemplary TGF-beta Inhibitors appear in Table 5. TGF-beta type I receptor inhibitors include but are not limited to 2-(3-(6-Methylpyridin-2-yl)-1H-pyrazol-4-yl)-1,5 napththyridine, [3-(Pyridin-2-yl)-4-(4-quinoyl)]-1H-pyrazole, and 3-(6-Methylpyridin-2-yl)-4-(4-quinolyl)-1-phenylthiocarbamoyl-1H-pyrazole, which can be purchased from Calbiochem (San Diego, Calif.). Other small molecule inhibitors include, but are not limited to, SB-431542 (see e.g., Halder et al., 2005; Neoplasia 7(5):509-521), SM16 (see e.g., Fu, K et al., 2008; Arteriosclerosis, Thrombosis and Vascular Biology 28(4):665), and SB-505124 (see e.g., Dacosta Byfield, S., et al., 2004; Molecular Pharmacology 65:744-52), among others.

TABLE 5
TGF-beta Inhibitors

| Class | Agent | CAS Number | Alternative Name |
|---|---|---|---|
| Tgf-beta-R1 inhibitor | LY-364947 | 396129-53-6 | 616451, TGF-β RI Kinase Inhibitor I, [3- |
| | | | (Pyridin-2-yl)-4-(4-quinonyl)]-1H-pyrazole, |
| | | | ALK5 Inhibitor I, LY-364947, HTS-466284 |
| Tgf-beta-R1 inhibitor | Repsox | 446859-33-2 | 616452, TGF-β RI Kinase Inhibitor II, 2-(3- |
| | | | (6-Methylpyridin-2-yl)-1H-pyrazol-4-yl)- |
| | | | 1,5-naphthyridine |
| Tgf-beta-R1 inhibitor | SB-505124 | 356559-13-2 | 616453, TGF-β RI Kinase Inhibitor III, |
| | | | CAS 356559-13-2 2-(5-Benzo[1,3]dioxol- |
| | | | 4-yl-2-tert-butyl-1H-imidazol-4-yl)-6- |
| | | | methylpyridine, HCl, ALK5 Inhibitor III, |
| Tgf-beta-R1 inhibitor | A-83-01 | 909910-43-6 | 616454, TGF-β RI Kinase Inhibitor IV-3- |
| | | | (6-Methylpyridin-2-yl)-4-(4-quinolyl)-1- |
| | | | phenylthiocarbamoyl-1H-pyrazole, A-83- |
| | | | 01, ALK5 Inhibitor IV |
| Tgf-beta-R1 inhibitor | SD-208 | 627536-09-8 | 616456, TGF-β RI Kinase Inhibitor V, 2-(5- |
| | | | Chloro-2-fluorophenyl)pteridin-4- |
| | | | yl)pyridin-4-yl amine, SD-208, ALK5 |
| | | | Inhibitor V |
| Tgf-beta-R1 inhibitor | SB-431542 | 301836-41-9 | 616461, TGF-β RI Kinase Inhibitor VI, 4- |
| | | | [4-(3,4-Methylenedioxyphenyl)-5-(2- |
| | | | pyridyl)-1H-imidazol-2-yl]benzamide, |
| | | | Dihydrate, 4-[4-(1,3-Benzodioxol-5-yl)-5- |
| | | | (2-pyridyl)-1H-imidazol-2-yl]benzamide, |
| | | | Dihydrate |
| Tgf-beta-R1 inhibitor | TGF-β RI Kinase | 666729-57-3 | 616458, TGF-β RI Kinase Inhibitor VII, 1- |
| | Inhibitor VII | | (2-((6,7-Dimethoxy-4-quinolyl)oxy)-(4,5- |
| | | | dimethylphenyl)-1-ethanone, ALK5 |
| | | | Inhibitor VII |
| Tgf-beta-R1 inhibitor | SB-525334 | 356559-20-1 | 616459, TGF-β RI Kinase Inhibitor VIII- |
| | | | SB-525334, 6-(2-tert-Butyl-5-(6-methyl- |
| | | | pyridin-2-yl)-1H-imidazol-4-yl)- |
| | | | quinoxaline, ALK5 Inhibitor VIII |
| Tgf-beta-R1 inhibitor | TGF-β RI Kinase | 1117684-36-2 | 616463, TGF-β RI Kinase Inhibitor IX, 4- |
| | Inhibitor IX | | ((4-((2,6-Dimethylpyridin-3-yl)oxy)pyridin- |
| | | | 2-yl)amino)benzenesulfonamide, ALK5 |
| | | | Inhibitor IX |
| Tgf-beta-R1 inhibitor | GW788388 | 452342-67-5 | 4-(4-(3-(pyridin-2-yl)-1H-pyrazol-4- |
| | | | yl)pyridin-2-yl)-N-(tetrahydro-2H-pyran-4- |
| | | | yl)benzamide |
| Tgf-beta-R1 inhibitor | LY2109761 | 700874-71-1 | 7-(2-morpholinoethoxy)-4-(2-(pyridin-2- |
| | | | yl)-5,6-dihydro-4H-pyrrolo[1,2-b]pyrazol- |
| | | | 3-yl)quinoline |
| Tgf-beta-R1 inhibitor | Galunisertib | 700874-72-2 | 4-(2-(6-methylpyridin-2-yl)-5,6-dihydro- |
| | (LY2157299) | | 4H-pyrrolo[1,2-b]pyrazol-3-yl)quinoline-6- |
| | | | carboxamide |
| Tgf-beta-R1 inhibitor | EW-7197 | 1352608-82-2 | N-(2-fluorophenyl)-5-(6-methyl-2- |
| | | | pyridinyl)-4-[1,2,4]triazolo[1,5-a]pyridin-6- |
| | | | yl-1H-imidazole-2-methanamine |
| Tgf-beta production | Pirfenidone | 53179-13-8 | 5-methyl-1-phenyl-2(1H)-Pyridinone, |
| inhibitor | | | |
| Tgf-beta-R1 inhibitor | K02288 | 1431985-92-0 | 3-[(6-Amino-5-(3,4,5-trimethoxyphenyl)-3- |
| | | | pyridinyl]phenol |
| Tgf-beta-R1 inhibitor | D 4476 | 301836-43-1 | 4-[4-(2,3-Dihydro-1,4-benzodioxin-6-yl)-5- |
| | | | (2-pyridinyl)-1H-imidazol-2-yl]benzamide |
| Tgf-beta-R1 inhibitor | R 268712 | 879487-87-3 | 4-[2-Fluoro-5-[3-(6-methyl-2-pyridinyl)- |
| | | | 1H-pyrazol-4-yl]phenyl]-1H-pyrazole-1- |
| | | | ethanol |
| Other | ITD 1 | 1099644-42-4 | 4-[1,1′-Biphenyl]-4-yl-1,4,5,6,7,8- |
| | | | hexahydro-2,7,7-trimethyl-5-oxo-3- |
| | | | quinolinecarboxylic acid ethyl ester |
| Smad3 inhibitor | SIS3 | 1009104-85-1 | 1,2,3,4-Tetrahydro-6,7-dimethoxy-2-[(2E)- |
| | | | 3-(1-phenyl-1H-pyrrolo[2,3-b]pyridin-3- |
| | | | yl)-1-oxo-2-propenyl]-isoquinoline |
| | | | hydrochloride |
| Tgf-beta-R1 inhibitor | A77-01 | 909910-42-5 | 4-[5-(6-methylpyridin-2-yl)-1H-pyrazol-4- |
| | | | yl]quinoline |
| Tgf-beta-R1 inhibitor | SM16 | 614749-78-9 | 4-(5-(benzo[d][1,3]dioxol-5-yl)-4-(6- |
| | | | methylpyridin-2-yl)-1H-imidazol-2- |
| | | | yl)bicyclo[2.2.2]octane-1-carboxamide |
| Tgf-beta-R1 inhibitor | LY-550410 | 737791-20-7 | CAS 737791-20-7 4-[5,6-dihydro-2-(2- |
| | | | pyridinyl)-4H-pyrrolo[1,2-b]pyrazol-3-yl]- |
| | | | Quinoline |
| Tgf-beta-R1 inhibitor | LY-580276 | 476475-07-7 | CAS 476475-07-7 3-(4-fluorophenyl)-5,6- |
| | | | dihydro-2-(6-methyl-2-pyridinyl)-4H- |
| | | | Pyrrolo[1,2-b]pyrazole |
| Tgf-beta-R1 inhibitor | EW-7203 | 1383123-98-5 | 1383123-98-5 3-[[[4-(6-methyl-2- |
| | | | pyridinyl)-5-[1,2,4]triazolo[1,5-a]pyridin-6- |
| | | | yl-2-thiazolyl]amino]methyl]-Benzonitrile, |
| Tgf-beta-R1 inhibitor | EW-7195 | 1352609-28-9 | 3-[[[5-(6-methyl-2-pyridinyl)-4-[1,2,4]triazolo[1, |
| | | | 5-a]pyridin-6-yl-1H-imidazol-2- |
| | | | yl]methyl]amino]-Benzonitrile |
| Tgf-beta-R1 inhibitor | GW6604 | 452342-37-9 | Pyridine, 2-phenyl-4-[3-(2-pyridinyl)-1H- |
| | | | pyrazol-4-yl]- |
| Tgf-beta-R1 inhibitor | Cmpd 3d | 733806-89-8 | 4-Quinazolinamine, 2-(6-methyl-2- |
| | | | pyridinyl)-N-4-pyridinyl- |
| Tgf-beta-R1 inhibitor | LY-566578 | 607738-00-1 | Pyridine, 2-[4-(4-fluorophenyl)-1H-pyrazol- |
| | | | 3-yl]-6-methyl- |
| Tgf-beta-R1 inhibitor | Cmpd 5 | 607738-02-3 | Phenol, 4-[3-(6-methyl-2-pyridinyl)-1H- |
| | | | pyrazol-4-yl] |
| Tgf-beta-R1 inhibitor | Cmpd 3 | 676331-30-9 | Quinoline, 7-ethoxy-4-[3-(2-pyridinyl)-1H- |
| | | | pyrazol-4-yl]- |
| Tgf-beta-R1 inhibitor | Cmpd 8b | 705263-50-9 | 1H-Benzimidazole, 6-[5,6-dihydro-2-(2- |
| | | | pyridinyl)-4H-pyrrolo[1,2-b]pyrazol-3-yl]- |
| Tgf-beta-R1 inhibitor | Cmpd 4b | 1308760-90-8 | N-(3-cyanophenyl)-3-(6-methyl-2- |
| | | | pyridinyl)-4-(6-quinolinyl)-1H-Pyrazole-1- |
| | | | acetamide |
| Tgf-beta-R1 inhibitor | Cmpd 21b | 1607465-38-2? | 1H-Pyrazole-1-acetamide, N-(3- |
| | | | cyanophenyl)-3-(6-methyl-2-pyridinyl)-4- |
| | | | [1,2,4]triazolo[1,5-a]pyridin-6-yl |
| Tgf-beta-R1 inhibitor | PF-03671148 | 1378524-25-4 | 3-methyl-6-[1-(6-methyl-2-pyridinyl)-1H- |
| | | | pyrazol-5-yl]-4(3H)-Quinazolinone, |
| Tgf-beta-R1 inhibitor | SB-203580 | 152121-47-6 | Pyridine, 4-[4-(4-fluorophenyl)-2-[4- |
| | | | (methylsulfinyl)phenyl]-1H-imidazol-5-yl]- |
| Tgf-beta-R1 inhibitor | SB-202190 | 152121-30-7 | 4-[4-(4-Fluorophenyl)-5-(4-pyridinyl)-1H- |
| | | | imidazol-2-yl]phenol |
| Tgf-beta-R1 inhibitor | IN-1130 | 868612-83-3 | 3-[[5-(6-methyl-2-pyridinyl)-4-(6- |
| | | | quinoxalinyl)-1H-imidazol-2-yl]methyl]- |
| | | | Benzamide, |
| Tgf-beta-R1 inhibitor | IN-1233 | 1093952-95-4 | 3-[[5-(6-methyl-2-pyridinyl)-4-(6- |
| | | | quinolinyl)-1H-imidazol-2-yl]methyl]- |
| | | | Benzamide, |
| Tgf-beta-R1 inhibitor | Cmpd 16i | 864375-44-0 | [[4-(6-benzothiazolyl)-5-(4-methyl-2- |
| | | | thiazolyl)-1H-imidazol-2-yl]methyl]-2- |
| | | | methylpropyl ester Carbamic acid |
| Tgf-beta-R1 inhibitor | LDN-214117 | 1627503-67-6 | 1-[4-[6-methyl-5-(3,4,5-trimethoxyphenyl)- |
| | | | 3-pyridinyl]phenyl]-Piperazine |
| Tgf-beta-R1 inhibitor | LDN-193189 | 1627503-67-6 | CAS 1062368-24-4, 4-[6-[4-(1-piperazinyl)phenyl]pyrazolo[1, |
| | | | 5-a]pyrimidin-3-yl]- |
| | | | Quinoline |
| Tgf-beta-R1 inhibitor | Cmpd 12b | 1415663-82-9 | 2-N-[(3-fluorophenyl)methyl]-4-(6-methyl- |
| | | | 2-pyridinyl)-5-[1,2,4]triazolo[1,5-a]pyridin- |
| | | | 6-yl Thiazolamine |
| Tgf-beta-R1 inhibitor | Cmpd 6d | 1630024-29-1 | 5-[[2-cyclopropyl-6-(4-fluorophenyl)imidazo[2, |
| | | | 1-b]-1,3,4-thiadiazol-5-yl]methylene]- |
| | | | 4-oxo-2-thioxo-3- |
| | | | Thiazolidineacetic acid |
| Tgf-beta-R1 inhibitor | SD-093 | | Structure unknown |
| Tgf-beta-R1 inhibitor | Ki-26894 | | Structure unknown |
| Tgf-beta-R1 inhibitor | NPC-30345 | | Structure unknown |
| Tgf-beta-R1 inhibitor | SX-007 | | Structure unknown |
| Tgf-beta-R1 inhibitor | SKI-2162 | | Structure unknown |
| Other | Asiaticoside | 16830-15-2 | |
| Tgf-beta antibody | ID11 | | |
| Tgf-beta antibody | 2G7 | | |
| Tgf-beta antibody | GC-1008 | | Fresolimumab |
| Tgf-beta antibody | CAT-152 | | Lerdelimimab |
| Tgf-beta antibody | CAT-192 | | Metelimumab |
| TGf-beta Receptor | PF-03446962 | | |
| antibody | | | |
| Tgf-beta antibody | SR-2F | | |
| Tgf-beta antibody | 2G7 | | |
| Tgf-beta antibody | LY2382770 | | |
| Tgf-beta antibody | IMC-TR1 | | |
| Tgf-beta antibody | STX-100 | | |
| TGF-beta antagonist | TGF-PRII:Fc | | |
| Recombinant protein | betaglycan/TGF- | | |
| | PRIII | | |
| Oligonucleotide | AP12009 | | Trabedersen, antisense molecule |
| inhibitor | | | |
| Oligonucleotide | AP11014 | | |
| inhibitor | | | |
| Oligonucleotide | AP15012 | | |
| inhibitor | | | |
| Is this TGF b | LY-573636 | 519055-62-0 | N-[(5-bromo-2-thienyl)sulfonyl]-2,4- |
| inhibitor/YES | | | dichloro-Benzamide |
| | pyrrole-imidazole | | Gene silencing |
| | polyamide | | |
| | U.S. Pat. No. | | Pyrrole derivatives as pharmaceutical |
| | 7,087,626 | | agents |
| | U.S. Pat. No. | | Quinazoline derivatives as medicaments |
| | 6,476,031 | | |
| | U.S. Pat. No. | | Antibodies to TGF-β |
| | 7,723,486, and | | |
| | EP 0945464 | | |
| Peptide | Tryptopeptin A | 1644153-72-9 | |
| Peptide | Trx-xFoxHlb | | Smad-interacting peptide aptamers |
| Peptide | Trx-Lefl | | |
| Peptide | Distertide (pl44) | | |
| Peptide | pl7 | | |
| Peptide | LSKL | | |
| dihydropyrrlipyrazole- | See US Patent U.S. Pat. No. | | |
| based scaffold | 8,298,825 B1 | | |
| imidazole-based | See US Patent U.S. Pat. No. | | |
| scaffold | 8,298,825 B1 | | |
| pyrazolopyridine- | See US Patent U.S. Pat. No. | | |
| based scaffold | 8,298,825 B1 | | |
| pyrazole-based | | | See US Patent U.S. Pat. No. 8,298,825 B1 |
| scaffold | | | |
| imidazopyridine-based | See US Patent U.S. Pat. No. | | |
| scaffold | 8,298,825 B1 | | |
| triazole-based scaffold | | | See US Patent U.S. Pat. No. 8,298,825 B1 |
| pyridopyrimidine- | See US Patent U.S. Pat. No. | | |
| based scaffold | 8,298,825 B1 | | |
| pyrrolopyrazole-based | See US Patent U.S. Pat. No. | | |
| scaffold | 8,298,825 B1 | | |
| isothiazole-based | See US Patent U.S. Pat. No. | | |
| scaffold | 8,298,825 B1 | | |
| oxazole-based scaffold | See US Patent U.S. Pat. No. | | |
| | 8,298,825 B1 | | |

In certain embodiments, the TGF-beta inhibitor of the present disclosure is selected from: Galunisertib (LY2157299) {4-(2-(6-methylpyridin-2-yl)-5,6-dihydro-4H-pyrrolo[1,2-b]pyrazol-3-yl)quinoline-6-carboxamide}, EW-7197 {N-(2-fluorophenyl)-5-(6-methyl-2-pyridinyl)-4-[1,2,4]triazolo[1,5-a]pyridin-6-yl-1H-imidazole-2-methanamine}, IN-1130 {3-[[5-(6-methyl-2-pyridinyl)-4-(6-quinoxalinyl)-1H-imidazol-2-yl]methyl]-Benzamide}, EW-7203 {3-[[[4-(6-methyl-2-pyridinyl)-5-[1,2,4]triazolo[1,5-a]pyridin-6-yl-2-thiazolyl]amino] methyl]-Benzonitrile}, EW-7195 {3-[[[5-(6-methyl-2-pyridinyl)-4-[1,2,4]triazolo[1,5-a] pyridin-6-yl-1H-imidazol-2-yl]methyl]amino]-Benzonitrile}, Repsox {2-(3-(6-Methylpyridin-2-yl)-1H-pyrazol-4-yl)-1,5-naphthyridine}, SM16 {4-(5-(benzo[d][1,3]dioxol-5-yl)-4-(6-methylpyridin-2-yl)-1H-imidazol-2-yl)bicyclo[2.2.2]octane-1-carboxamide}, R 268712 {4-[2-Fluoro-5-[3-(6-methyl-2-pyridinyl)-1H-pyrazol-4-yl]phenyl]-1H-pyrazole-1-ethanol}, GW788388 {4-(4-(3-(pyridin-2-yl)-1H-pyrazol-4-yl)pyridin-2-yl)-N-(tetrahydro-2H-pyran-4-yl)benzamide}, and PF-03671148 {3-methyl-6-[1-(6-methyl-2-pyridinyl)-1H-pyrazol-5-yl]-4(3H)-quinazolinone}, SB-431542, A-83-01 {3-(6-Methylpyridin-2-yl)-4-(4-quinolyl)-1-phenylthiocarbamoyl-1H-pyrazole}, A77-01 {4-[5-(6-methylpyridin-2-yl)-1H-pyrazol-4-yl]quinolone}, SB-525334 {6-(2-tert-Butyl-5-(6-methyl-pyridin-2-yl)-1H-imidazol-4-yl)-quinoxaline}, Cmpd 16i {[[4-(6-benzothiazolyl)-5-(4-methyl-2-thiazolyl)-1H-imidazol-2-yl]methyl]-2-methylpropyl ester Carbamic acid}, Cmpd 12b {2-N-[(3-fluorophenyl)methyl]-4-(6-methyl-2-pyridinyl)-5-[1,2,4]triazolo[1,5-a]pyridin-6-yl thiazolamine}, Cmpd 6d {5-[[2-cyclopropyl-6-(4-fluorophenyl)imidazo[2,1-b]-1,3,4-thiadiazol-5-yl]methylene]-4-oxo-2-thioxo-3-Thiazolidineacetic acid}, and Pirfenidone {5-methyl-1-phenyl-2(1H)-Pyridinone}.

In certain embodiments, the TGF-beta inhibitor is selected from: Galunisertib (LY2157299), EW-719, IN-1130, EW-7203, EW-7195, Repsox, SM16, R 268712, GW788388, SB-431542, A-83-01, and PF-03671148.

In certain embodiments, the TGF-beta inhibitor is selected from: Galunisertib (LY2157299), EW-7197, IN-1130, EW-7203, EW-7195, SB-431542, A-83-01, and Repsox.

Classes of TGF-beta inhibitors for use in various embodiments of the compositions and methods disclosed herein include but are not limited to those listed in Column A of Table 5. Specific TGF-beta inhibitors for use in various embodiments of the compositions and methods disclosed herein include but are not limited to those listed in Column B of Table 5. All agents listed in Table 5 column B are understood to include derivatives or pharmaceutically-acceptable salts thereof. All classes listed in Table 5 column A are understood to include both agents comprising that class and derivatives or pharmaceutically-acceptable salts thereof.

Exemplary GSK3-alpha inhibitors within the present disclosure appear in Table 6.

TABLE 6
GSK3-alpha Inhibitors

| | | | Potency in | Potency | |
|---|---|---|---|---|---|
| | | | nM | in nM | Ratio of |
| Column A | Column B | | GSK3- | GSK3- | Alpha to |
| Class | Agent | CAS Number | alpha | beta | Beta |
| Pyrazole | GSK-3b XXII | 1195901-31-5 | 2.3 | 2.0 | 0.87 |
| Pyrazole | AT 7519 | 844442-38-2 | | 89 | |
| Pyrazole | Compound 4a | 1627557-91-8 | | 8 | |
| Pyrazole | Compound 4t | 1627558-10-4 | | <5 | |
| Pyrazole | Compound 4z | 1627558-16-0 | | 5 | |
| Pyrazolopyridines | Compound 14 | 583038-63-5 | 1 | | |
| Pyrazolopyridines | Compound 23 | 583038-76-0 | 1 | | |
| Pyrazolopyridines | Pyrazolopyridine 34 | 583039-27-4 | 7 | | |
| Pyrazolopyridazines | Compound 18 | 405223-20-3 | 0.95 | | |
| Pyrazolopyridazines | Compound 19 | 405223-71-4 | 0.19 | | |
| Oxadiazoles | Compound 15b | 1374671-66-5 | 2 (230) | 185 (>1K) | 92 (>4.3) |
| Oxadiazoles | Compound 14d | 1374671-64-3 | 6 | 316 | 52 |
| Oxadiazoles | Compound 27 | 1820758-44-8 | 42 | 140 | 3.3 |
| Oxindole | AZD1080 | 612487-72-6 | 6.9 | 31 | 4.5 |
| Isonicotinamides | Compound 39 | 1772824-10-8 | 0.34 | 1.9 | 5.6 |
| Isonicotinamides | Compound 29 | 1772823-37-6 | 1.7 | 5.2 | 3.0 |
| Isonicotinamides | Compound 33 | 1772823-64-9 | 2 | 5.9 | 2.9 |
| Maleimide | Tivantinib | 905854-02-6 | 659 | 1865 | 2.8 |
| Maleimide | I5 | 264217-24-5 | 76 | 160 | 2.1 |
| Triazolpyrimidine | Compound 90 | 91322-11-1 | 330 | 628 | 1.9 |
| Triazolpyrimidine | Compound 92 | 1043429-30-6 | 9 | 13 | 1.4 |
| Organometallic | Compound lambda- | 1291104-51-2 | 0.9 | 6 | 6.8 |
| | OS1 | 1292843-11-8 | | | |
| Organometallic | Compound 3 | 1498285-39-4 | 3 | 10 | 3.3 |
| | | 1498285-48-5 | | | |
| Organometallic | Compound (R)-DW12 | 1047684-07-0 | 0.5 | 1 | 2 |
| Pyrazolo- | BRD4003 chiral | 1597439-60-5 | 4800 | 10,200 | 2.1 |
| tetrahydroquinolinone | | | | | |
| Pyrazolo- | BRD4003 chiral | 1597439-59-2 | 161 | 232 | 1.4 |
| tetrahydroquinolinone | | | | | |
| Pyrazolo- | Compound 8 | 1597439-01-4 | 18 | 87 | 4.8 |
| tetrahydroquinolinone | | | | | |
| Pyrazolo- | Compound 9 | 1597439-02-5 | 62 | 156 | 2.5 |
| tetrahydroquinolinone | | 2056261-29-9 | | | |
| Pyrazolo- | Compound 11 | 1597439-12-7 | 32 | 102 | 3.2 |
| tetrahydroquinolinone | | | | | |
| Pyrazolo- | BRD1172 | 1597438-86-2 | 3 | 10 | 3.3 |
| tetrahydroquinolinone | | | | | |
| Pyrazolo- | Compound 16 | 1597440-17-9 | 8 | 26 | 3.2 |
| tetrahydroquinolinone | | | | | |
| Pyrazolo- | BRD1652 | 1597438-93-1 | 0.4 | 4 | 10 |
| tetrahydroquinolinone | | | | | |
| Urea | AR-A014418 | 487021-52-3 | 28 | 116 | 4.1 |
| CREB knockdown | | | | | |

ACS Chem. Biol. 2016, 11, 1952-1963
PLoS One (2016), 11(4), e0153075

### Additional Therapeutic Agents

In certain embodiments, the administering step comprises administering or causing to be administered to the stem cell population one or more additional agents (e.g., in addition to a Wnt agonist, GSK3-alpha inhibitor, or GSK3-beta inhibitor and a TGF-beta inhibitor.

In certain embodiments, the one or more additional agents comprises a TGF-beta type I receptor inhibitor. TGF-beta type I receptor inhibitors include but are not limited to 2-(3-(6-Methylpyridin-2-yl)-1H-pyrazol-4-yl)-1,5 napththyridine, [3-(Pyridin-2-yl)-4-(4-quinoyl)]-1H-pyrazole, and 3-(6-Methylpyridin-2-yl)-4-(4-quinolyl)-1-phenylthiocarbamoyl-1H-pyrazole, which can be purchased from Calbiochem (San Diego, Calif.). Other small molecule inhibitors include, but are not limited to, SB-431542 (see e.g., Halder et al., 2005; Neoplasia 7(5):509-521), SM16 (see e.g., Fu, K et al., 2008; Arteriosclerosis, Thrombosis and Vascular Biology 28(4):665), and SB-505124 (see e.g., Dacosta Byfield, S., et al., 2004; Molecular Pharmacology 65:744-52), among others.

In one embodiment, the ALK5 inhibitor 2-(3-(6-Methylpyridin-2-yl)-1H-pyrazol-4-yl)-1,5 napththyridine is used with the methods described herein. This inhibitor is also referred to herein as ALK5 inhibitor II and is available commercially from Calbiochem (Cat. No. 616452; San Diego, Calif.). In one embodiment, the inhibitor is SB-431542, an ALK-4, -5, -7inhibitor, commercially available from Sigma (product no. 54317; Saint Louis, Mo.). SB-431542 is also referred to by the following chemical names: 4-[4-(1,3-Benzodioxol-5-yl)-5-(2-pyridinyl)-1H-imidazol-2-yl]-benzamide, 4-[4-(3,4-methylenedioxyphenyl)-5-(2-pyridyl)-1H-imidazol-2-yl]-benzamide, or 4-(5-benzol[1,3]dioxol-5-yl-4-pyridin-2-yl-1H-imidazol-2-yl)-benzamide hydrate.

Small molecules inhibitors of TGF-β signaling can be classified based on the basic scaffold of the molecule. For example, TGF-β signaling inhibitors can be based on the dihydropyrrlipyrazole-based scaffold, imidazole-based scaffold, pyrazolopyridine-based scaffold, pyrazole-based scaffold, imidazopyridine-based scaffold, triazole-based scaffold, pyridopyrimidine-based scaffold, pyrrolopyrazole-based scaffold, isothiazole-based scaffold and oxazole-based scaffold.

Inhibitors of TGF-β signaling are described, for example, in Callahan, J. F. et al., J. Med. Chem. 45, 999-1001 (2002); Sawyer, J. S. et al., J. Med. Chem. 46, 3953-3956 (20031; Gellibert, F. et al., J. Med. Chem. 47, 4494-4506 (2004); Tojo, M. et al., Cancer Sci. 96: 791-800 (2005); Valdimarsdottir, G. et al., APMIS 113, 773-389 (2005); Petersen et al. Kidney International 73, 705-715 (2008); Yingling, J. M. et al., Nature Rev. Drug Disc. 3, 1011-1022 (2004); Byfield, S. D. et al., Mol. Pharmacol., 65, 744-752 (2004); Dumont, N, et al., Cancer Cell 3, 531-536 (2003); WO Publication No. 2002/094833; WO Publication No. 2004/026865; WO Publication No. 2004/067530; WO Publication No. 209/032667; WO Publication No. 2004/013135; WO Publication No. 2003/097639; WO Publication No. 2007/048857; WO Publication No. 2007/018818; WO Publication No. 2006/018967; WO Publication No. 2005/039570; WO Publication No. 2000/031135; WO Publication No. 1999/058128; U.S. Pat. No. 6,509,318; U.S. Pat. No. 6,090,383; U.S. Pat. No. 6,419,928; U.S. Pat. No. 9,927,738; U.S. Pat. No. 7,223,766; U.S. Pat. No. 6,476,031; U.S. Pat. No. 6,419,928; U.S. Pat. No. 7,030,125; U.S. Pat. No. 6,943,191; U.S. Publication No. 2005/0245520; U.S. Publication No. 2004/0147574; U.S. Publication No. 2007/0066632; U.S. Publication No. 2003/0028905; U.S. Publication No. 2005/0032835; U.S. Publication No. 2008/0108656; U.S. Publication No. 2004/015781; U.S. Publication No. 2004/0204431; U.S. Publication No. 2006/0003929; U.S. Publication No. 2007/0155722; U.S. Publication No. 2004/0138188 and U.S. Publication No. 2009/0036382, the contents of each which are herein incorporated by reference in their entirety.

Oligonucleotide based modulators of TGF-β signaling, such as siRNAs and antisense oligonucleotides, are described in U.S. Pat. No. 5,731,424; U.S. Pat. No. 6,124,449; U.S. Publication Nos. 2008/0015161; 2006/0229266; 2004/0006030; 2005/0227936 and 2005/0287128, each of which are herein incorporated by reference in their entirety. Other antisense nucleic acids and siRNAs can be obtained by methods known to one of ordinary skill in the art.

Exemplary inhibitors of TGF-β signaling include, but are not limited to, AP-12009 (TGF-β Receptor type II antisense oligonucleotide), Lerdelimumab (CAT 152, antibody against TGF-β Receptor type II) GC-1008 (antibody to all isoforms of human TGF-β), ID11 (antibody to all isoforms of murine TGF-β), soluble TGF-β, soluble TGF-β Receptor type II, dihydropyrroloimidazole analogs (e.g., SKF-104365), triarylimidazole analogs (e.g., SB-202620 (4-(4-(4-fluorophenyl)-5-(pyridin-4-yl)-1H-imidazol-2-yl)benzoic acid) and SB-203580 (4-(4-Fluorophenyl)-2-(4-methylsulfinyl phenyl)-5-(4-pyridyl)-1H-imidazole)), RL-0061425, 1,5-naphthyridine aminothiazole and pyrazole derivatives (e.g., 4-(6-methyl-pyridin-2-yl)-5-(1,5-naphthyridin-2-yl)-1,3-thiazole-2-amine and 2-[3-(6-methyl-pyridin-2-yl)-1H-pyrazole-4-yl]-1,5-naphthyridine), SB-431542 (4-(5-Benzol[1,3]dioxol-5-yl-4-pyridin-2-yl-1H-imidazol-2-yl)-benzamide), GW788388 (4-(4-(3-(pyridin-2-yl)-1H-pyrazol-4-yl)pyridin-2-yl)-N-(tetrahydro-2H-pyran-4-yl)benzamide), A-83-01 (3-(6-Methyl-2-pyridinyl)-N-phenyl-4-(4-quinolinyl)-1H-pyrazole-1-carbothioamide), Decorin, Lefty 1, Lefty 2, Follistatin, Noggin, Chordin, Cerberus, Gremlin, Inhibin, BIO (6-bromo-indirubin-3′-oxime), Smad proteins (e.g., Smad2, Smad3), and Cystatin C.

Inhibitors of TGF-β signaling also include molecules which inhibit TGF-β Receptor type I. Inhibitors of TGF-β Receptor type I are described in Byfield, S. D., and Roberts, A. B., Trends Cell Biol. 14, 107-111 (2004); Sawyer J. S. et al., Bioorg. Med. Chem. Lett. 14, 3581-3584 (2004); Sawyer, J. S. et al., J. Med. Chem. 46, 3953-3956 (2003); Byfield, S. D. et al., Mol. Pharmacol. 65, 744-752 (2004); Gellibert, F. et al., J. Med. Chem. 47, 4494-4506 (2004); Yingling, J. M. et al., Nature Rev. Drug Disc. 3, 1011-1022 (2004); Dumont, N, et al., Cancer Cell 3, 531-536 (2003); Tojo, M. et al., Cancer Sci. 96: 791-800 (2005); WO Publication No. 2004/026871; WO Publication No. 2004/021989; WO Publication No. 2004/026307; WO Publication No. 2000/012497; U.S. Pat. No. 5,731,424; U.S. Pat. No. 5,731,144; U.S. Pat. No. 7,151,169; U.S. Publication No. 2004/00038856 and U.S. Publication No. 2005/0245508, contents of all of which are herein incorporated in their entireties.

In certain embodiments, the stem cell population is of an in vivo subject, and the method is a treatment for hearing loss and/or vestibular dysfunction (e.g., wherein the generation of inner ear hair cells from the expanded population of stem cells results in partial or full recovery of hearing loss and/or improved vestibular function). In certain embodiments, the stem cell population is of an in vivo subject, and the method further comprises delivering a drug to the subject (e.g., for treatment of a disease and/or disorder unrelated to hearing loss and/or vestibular dysfunction) at a higher concentration than a known safe maximum dosage of the drug for the subject (e.g., the known safe maximum dosage if delivered in the absence of the generation of inner ear hair cells resulting from the method) (e.g., due to a reduction or elimination of a dose-limiting ototoxicity of the drug).

In certain embodiments, the method further comprises performing high throughput screening using the generated inner ear hair cells. In certain embodiments, the method comprises using the generated inner ear hair cells to screen molecules for toxicity against inner ear hair cells. In certain embodiments, the method comprises using the generated inner ear hair cells to screen molecules for ability to improve survival of inner ear hair cells (e.g., inner ear hair cells exposed to said molecules).

In some aspects, the disclosure is directed to a method of producing an expanded population of stem cells, the method comprising: administering or causing to be administered to a stem cell population (e.g., of an in vitro, ex vivo, or in vivo sample/subject) both of (i) and (ii): (i) a Wnt agonist, a GSK3-alpha inhibitor, or a GSK3-beta inhibitor, and (ii) a TGF-beta inhibitor, thereby proliferating stem cells in the stem cell population and resulting in an expanded population of stem cells. In certain embodiments, the stem cell population comprises supporting cells. In certain embodiments, the stem cell population comprises post-natal stem cells. In certain embodiments, the stem cell population comprises epithelial stem cells. In certain embodiments, stem cells include progenitor cells.

In certain embodiments, the administering step is carried out by performing one or more injections into the ear (e.g., transtympanically into the middle ear and/or inner ear).

In certain embodiments, the administering step comprises administering or causing to be administered to the stem cell population a Wnt agonist, a GSK3-alpha inhibitor, or a GSK3-beta inhibitor comprising a synthetic molecule.

In certain embodiments, the administering step is carried out by performing one or more injections into the ear (e.g., transtympanically into the middle ear and/or inner ear).

In certain embodiments, the administering step comprises administering the notch agonist and/or HDAC inhibitor in a pulsatile manner and administering the GSK3-beta inhibitor and/or Wnt agonist in a sustained manner.

In certain embodiments, the administering step comprises administering the notch agonist and/or HDAC inhibitor in a pulsatile manner and administering the GSK3-alpha inhibitor and/or Wnt agonist and/or a GSK3-beta inhibitor in a sustained manner.

In certain embodiments, the stem cells are inner ear stem cells and/or supporting cells.

In certain embodiments, the method further comprises performing high throughput screening using the generated expanded population of stem cells. In certain embodiments, the method further comprises using the generated stem cells to screen molecules for toxicity against stem cells and/or their progeny. In certain embodiments, the method comprises using the generated stem cells to screen molecules for ability to improve survival of stem cells and/or their progeny.

In some aspects, the disclosure is directed to a method of treating a subject who has, or is at risk of developing, hearing loss and/or vestibular dysfunction, the method comprising: identifying a subject who has experienced, or is at risk for developing, hearing loss and/or vestibular dysfunction, administering or causing to be administered (i) a Wnt agonist, a GSK3-alpha inhibitor, or a GSK3-beta inhibitor and (ii) a TGF-beta inhibitor.

In certain embodiments, the stem cell population comprises supporting cells. In certain embodiments, the stem cell population comprises post-natal cells. In certain embodiments, the stem cell population comprises epithelial stem cells. In certain embodiments, stem cells include progenitor cells.

In certain embodiments, the step of administering is carried out by performing one or more injections into the ear (e.g., transtympanically into the middle ear and/or inner ear).

In some aspects, the disclosure is directed to a method of generating inner ear hair cells, the method comprising: proliferating stem cells in an initial stem cell population (e.g., of an in vitro, ex vivo, or in vivo sample/subject), resulting in an expanded population of stem cells (e.g., such that the expanded population is a factor of at least 1.25, 1.5, 1.75, 2, 3, 5, 10, or 20 greater than the initial stem cell population); and facilitating generation of inner ear hair cells from the expanded population of stem cells.

In some aspects, the disclosure is directed to a method of generating inner ear hair cells, the method comprising administering a composition comprising(i) a Wnt agonist, a GSK3-alpha inhibitor, or a GSK3-beta inhibitor and (ii) a TGF-beta inhibitor (e.g., in a pharmaceutically-acceptable form (e.g., salt)) to a cell population in an inner ear of a subject, thereby facilitating generation of inner ear hair cells.

In some aspects, the disclosure is directed to a method of generating inner ear hair cells, the method comprising: proliferating post-natal supporting cells in an initial population (e.g., of an in vitro, ex vivo, or in vivo sample/subject), resulting in an expanded population of supporting cells (e.g., such that the expanded population is a factor of at least 1.25, 1.5, 1.75, 2, 3, 5, 10, or 20 greater than the initial stem cell population), said expanded population of supporting cells resulting in generation of inner ear hair cells. In certain embodiments, stem cells include progenitor cells. In some embodiments, the proliferation is induced by a Wnt agonist, GSK3-alpha inhibitor, or GSK3-beta inhibitor. In some embodiment, the proliferation is induced by a Wnt agonist, GSK3-alpha inhibitor, or GSK3-beta inhibitor and a TGF-beta inhibitor.

In some aspects, the disclosure is directed to a method of treating a disease or disorder, the method comprising: proliferating post-natal supporting epithelial cells in an initial population of a subject (in vivo), resulting in an expanded population of supporting epithelial cells (e.g., such that the expanded population is a factor of at least 1.25, 1.5, 1.75, 2, 3, 5, 10, or 20 greater than the initial post-natal supporting epithelial cell population). In some embodiments, the proliferation is induced by a Wnt agonist, GSK3-alpha inhibitor, or GSK3-beta inhibitor. In some embodiment, the proliferation is induced by a Wnt agonist, GSK3-alpha inhibitor, or GSK3-beta inhibitor and a TGF-beta inhibitor.

In some embodiments, supporting cells are differentiated into hair cells.

### Compositions and Administration

Certain embodiments relate to pharmaceutical, prophylactic, or therapeutic compositions, comprising a pharmaceutically-acceptable carrier and (i) a Wnt agonist, a GSK3-alpha inhibitor, or a GSK3-beta inhibitor and (ii) a TGF-β inhibitor, or a pharmaceutically-acceptable salt thereof. In some embodiments, as noted above, a composition is adapted for administration to the inner ear and/or middle ear, for example, local administration to the round window membrane or intratympanic or transtympanic administration, for example, to vestibular tissue.

Certain compositions further comprise an additional agent selected from a Notch activator, HDAC inhibitor, a BMP4 antagonist, Noggin (Inhibits BMP4), Sox2, Vitamin D (calcitriol), Vitamin B (nicotinomide), Vitamin A, Vitamin C (pVc). Lgr4, p38/MAPK inhibition, ROCK inhibition, and/or Alk4/7 inhibition.

Some compositions further comprise an epidermal growth factor (EGF), a fibroblast growth factor (FGF), an insulin-like growth factor (IGF), or a combination thereof.

The phrase “pharmaceutically-acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically-acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically-acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

Certain compositions comprise at least one biocompatible matrix. The term “biocompatible matrix” as used herein is a polymeric carrier that is acceptable for administration to humans for the release of therapeutic agents. In some instances, a biocompatible matrix may be a biocompatible gel or foam.

Certain compositions comprise at least on poloxamer. Poloxamers are triblock copolymers formed of (i.e., hydrophilic poly(oxyethylene) blocks and hydrophobic poly(oxypropylene) blocks) configured as a triblock of poly(oxyethylene)-poly(oxypropylene)-poly(oxyethylene). Poloxamers are one class of block copolymer surfactants having a propylene oxide block hydrophobe and an ethylene oxide hydrophile. Poloxamers are commercially available (e.g., Pluronic® polyols are available from BASF Corporation). Alternatively, poloxamers can be synthesized by known techniques.

Exemplary poloxamers include Poloxamer 124, Poloxamer 188, Poloxamer 237, Poloxamer 338, and Poloxamer 407. In some embodiments, the poloxamer comprises mixtures of two or more of Poloxamer 124, Poloxamer 188, Poloxamer 237, Poloxamer 338 or Poloxamer 407. In some embodiments, the mixture of two or more poloxamers comprise Poloxamer 407 and Poloxamer 124. In certain embodiments the poloxamer comprises at least one of Poloxamer 188 and Poloxamer 407 or mixtures thereof. In some embodiments, the poloxamer is Poloxamer 407.

In some embodiments, the poloxamer is in a concentration between about 5 wt % and about 25 wt % relative to the composition, or about 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, or 25 wt % relative to the composition. In certain embodiments, the poloxamer is in a concentration between about 10 wt % and about 23 wt % relative to the composition. In some embodiments the poloxamer is in a concentration between about 15 wt % and about 20 wt % relative to the composition. In particular embodiments, the poloxamer is in a concentration is approximately 17 wt % relative to the composition.

In some embodiments, wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Certain compositions comprise at least one antioxidant. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In specific embodiments, the viscosity of the composition at about body temperature is substantially different (e.g., lesser, greater) than the viscosity of the composition at room temperature.

In some embodiments, the composition comprises a buffer. For example, in certain instances, the buffer is physiological saline or phosphate-buffered saline (PBS).

In some embodiments, the composition is at or near physiological pH. For instance, in some embodiments, the composition has a pH of between about 6 and about 8, including all integers, decimals, and ranges in between, for example, about 6 to about 6.5 to about 7 to about 7.5 to about 8. In specific embodiments, the composition has a pH of about 7.4 (±0.2).

In certain embodiments, the Wnt agonist, GSK3-alpha inhibitor, or GSK3-beta inhibitor is present in a pharmaceutical composition at an effective or otherwise defined concentration or concentration range. For example, in certain embodiments, the Wnt agonist, GSK3-alpha inhibitor, or GSK3-beta inhibitor is present in a composition at a concentration of about 0.01 uM to 1000 mM, about 0.1 uM to 1000 mM, about 1 uM to 100 mM, about 10 uM to 10 mM, about 1 uM to 10 uM, about 10 uM to 100 uM, about 100 uM to 1000 uM, about 1 mM to 10 mM, or about 10 mM to 100 mM; or at a concentration ratio of about 0.01 to 1,000,000 fold relative to its Effective Sternness Driver Concentration, or about 0.1 to 100,000 fold relative to the Effective Sternness Driver Concentration, or about 1 to 10,000 fold relative to the Effective Sternness Driver Concentration, or about 100 to 5000 fold relative to the Effective Sternness Driver Concentration, or about 50 to 2000 fold relative to the Effective Sternness Driver Concentration, or about 100 to 1000 fold relative to the Effective Sternness Driver Concentration, or at about 1000 fold relative to the Effective Sternness Driver Concentration; or at a concentration of about 0.01 nM to 1000 uM, about 0.1 nM to 1000 uM, about 1 nM to 100 uM, about 10 nM to 10 uM, about 1 nM to 10 nM, about 10 nM to 100 nM, about 100 nM to 1000 nM, about 1 uM to 10 uM, or about 10 uM to 100 uM, including all integers and ranges in between. In some embodiments, the Effective Sternness Driver Concentration is measured in an Lgr5 proliferation assay, as described herein.

In some embodiments, the Wnt agonist, GSK3-alpha inhibitor, or GSK3-beta inhibitor is CHIR99021, which is in the composition at a concentration of about 1 uM to 1000 mM, about 10 uM to 100 mM, about 100 uM to 100 mM, about 1 mM to 10 mM, or about 1, 2, 3, 4, 5, 6, 7, 8, 9, or 10 mM; or at a concentration of about 1 nM to 1000 uM, about 10 nM to 100 uM, about 100 nM to 100 uM, about 1 uM to 10 uM, or about 1, 2, 3, 4, 5, 6, 7, 8, 9, or 10 uM, including all integers and ranges in between.

In some embodiments, the Wnt agonist, GSK3-alpha inhibitor, or GSK3-beta inhibitor is LY2090314, which is in the composition at a concentration of about 0.01 uM to 1000 mM, about 0.1 uM to 10 mM, about 1 uM to 1 mM, about 10 uM, about 20 uM, about 30 uM, about 40 uM, or about 50 uM; or at a concentration of about 0.01 nM to 1000 uM, about 0.1 nM to 10 uM, about 1 nM to 1 uM, about 1 nM to 100 nM, or about 10 nM, including all integers and ranges in between.

In certain embodiments, the Wnt agonist, GSK3-alpha inhibitor, or GSK3-beta inhibitor is AZD1080, which is in the composition at a concentration of about 0.1 uM to 1000 mM, about 1 uM to 1000 mM, about 10 uM to 100 mM, about 100 uM to 10 mM, about 1 mM to 10 mM, or about 1, 2, 3, 4, 5, 6, 7, 8, 9, or 10 mM; or at a concentration of about 1 nM to 1000 uM, about 10 nM to 1000 uM, about 100 nM to 100 uM, about 1 uM to 10 uM, or about 1, 2, 3, 4, 5, 6, 7, 8, 9, or 10 uM, including all integers and ranges in between.

In certain embodiments, the Wnt agonist, GSK3-alpha inhibitor, or GSK3-beta inhibitor is GSK3 inhibitor XXII, which is in the composition at a concentration of about 0.1 uM to 1000 mM, about 1 uM to 100 mM, about 10 uM to 10 mM, about 100 uM to 10 mM, about 100 uM to 1 mM, or about 1, 2, 3, 4, 5, 6, 7, 8, 9, or 10 mM; or at a concentration of about 0.1 nM to 1000 uM, about 1 nM to 100 uM, about 10 nM to 10 uM, about 100 nM to 1 uM, or about 0.5 uM, including all integers and ranges in between.

In certain embodiments, the TGF-beta inhibitor is present in a pharmaceutical composition at an effective or otherwise defined concentration or concentration range. For instance, in some embodiments, the TGF-beta inhibitor is in the composition at a concentration of about 0.01 uM to 1000 mM, about 0.1 uM to 1000 mM, about 1 uM to 100 mM, about 0.1 uM to 1 uM, about 1 uM to 10 uM, about 10 uM to 100 uM, about 100 uM to 1 mM, about 1 mM to 10 mM, or about 100 mM to 1000 mM, or about 10 mM to 100 mM, or about 100 mM to 1000 mM; or at a concentration ratio of about 0.1 to 1,000,000 fold relative to its Effective TGF-beta Concentration, or about 1 to 100,000 fold relative to the Effective TGF-beta Concentration, or about 10 to 10,000 fold relative to the Effective TGF-beta Concentration, or about 100 to 1000 fold relative to the Effective TGF-beta Concentration, or about 1000 fold relative to the Effective TGF-beta Concentration; or at a concentration of about 0.01 nM to 1000 uM, or about0.1 nM to 1000 uM, about 1 nM to 100 uM, about 10 nM to 10 uM, about 1 nM to 10 nM, about 10 nM to 100 nM, about 100 nM to 1000 nM, about 1 uM to 10 uM, about 10 uM to 100 uM, or about 100 uM to 1000 uM, including all integers and ranges in between. In some embodiments, the Effective TGF-beta Concentration is measured in an Lgr5 proliferation assay, as described herein.

In some embodiments, the TGF-beta inhibitor is 616452 (Repsox) at a concentration of about 1 uM to 1000 mM, or about 10 uM to 1000 mM, or about 100 uM to 10 mM, or about 2 mM; or at a concentration of about 1 nM to 1000 uM, about 10 nM to 100 uM, about 100 nM to 10 uM, or about 2 uM, including all integers and ranges in between.

In certain embodiments, the BMP4 antagonist is present in a pharmaceutical composition at an effective or otherwise defined concentration or concentration range. For instance, in some embodiments, the BMP4 antagonist is in the composition at a concentration of about 0.01 uM to 1000 mM, 0.1 uM to 1000 mM, about 1 uM to 100 mM, about 10 uM to 10 mM, about 0.1 uM to 1 uM, about 1 uM to 10 uM, about 10 uM to 100 uM, about 100 uM to 1 mM, about 1 mM to 10 mM, about 10 mM to 100 mM, about 100 mM to 1000 mM; or at a concentration ratio of about 0.1 to 1,000,000 fold relative to its Effective BMP4 Antagonist Concentration, or about 1 to 100,000 fold relative to the Effective BMP4 Antagonist Concentration, or about 10 to 10,000 fold relative to the Effective BMP4 Antagonist Concentration, or about 100 to 1000 fold relative to the Effective BMP4 Antagonist Concentration, or about 1000 fold relative to the Effective BMP4 Antagonist Concentration; or at a concentration of about 0.01 nM to 100 uM, about 1 nM to 100 uM, about 10 nM to 10 uM, about 1 nM to 10 nM, about 10 nM to 100 nM, about 100 nM to 1000 nM, about 1 uM to 10 uM, about 10 uM to 100 uM, or about 100 uM to 1000 uM, including all integers and ranges in between. In some embodiments, the Effective BMP4 Antagonist Concentration is measured in an Lgr5 proliferation assay, as described herein.

In some embodiments, the BMP4 antagonist is DMH1 at a concentration of about 1 uM to 1000 mM, about 10 uM to 100 mM, about 100 uM to 10 mM, or about 1 mM; or at a concentration of about 1 nM to 1000 uM, or about 10 nM to 100 uM, about 100 nM to 10 uM, or about 1 uM, including all integers and ranges in between.

In certain embodiments, the BMP4 antagonist is Noggin at a concentration of about 1 ug/ml to 10,000 ug/ml, about 10 ug/ml to 1000 ug/ml, or about 100 ug/ml; or at a concentration of about 1 ng/ml to 10,000 ng/ml, about 10 ng/ml to 1000 ng/ml, or about 100 ng/ml, including all integers and ranges in between.

In certain embodiments, the HDAC inhibitor is present in a pharmaceutical composition at an effective or otherwise defined concentration or concentration range. For example, in certain embodiments the HDAC inhibitor is at a concentration of about 0.01 uM to 100,000 mM, about 1 uM to 10,000 mM, about 10 uM to 10,000 mM, about 100 uM to 1000 mM, about 1 uM to 10 uM, about 10 uM to 100 uM, about 100 uM to 1000 uM, about 1000 uM to 10 mM, about 10 mM to 100 mM, about 100 mM to 1000 mM, or about 1000 mM to 10,000 mM; or at a concentration ratio of about 0.1 to 1,000,000 fold relative to its Effective Concentration, or about 1 to 100,000 fold relative to the Effective Concentration, or about 10 to 10,000 fold relative to the Effective Concentration, or about 100 to 1000 fold relative to the Effective Concentration, or about 1000 fold relative to the Effective Concentration; or at a concentration of about 0.01 nM to 100,000 uM, about 1 nM to 10,000 uM, about 10 nM to 10,000 uM, about 100 nM to 1000 uM, about 1 nM to 10 nM, about 10 nM to 100 nM, about 100 nM to 1000 nM, about 1 uM to 10 uM, about 10 uM to 100 uM, about 100 uM to 1000 uM, or about 1000 uM to 10,000 uM, including all integers and ranges in between. In some embodiments, the Effective Concentration is measured in an Lgr5 proliferation assay, as described herein.

In some embodiments, the HDAC inhibitor is valproic acid at a concentration of about 10 uM to 100,000 mM, about 1 mM to 10,000 mM, about 10 mM to 10,000 mM, about 100 mM to 10,000 mM, about 200 mM to 2000 mM, about 1000 mM, or about 600 mM; or at a concentration of about 10 nM to 100,000 uM, 1 uM to 10,000 uM, about 10 uM to 10,000 uM, about 100 uM to 10,000 uM, about 200 uM to 2000 uM, or about 1000 uM, including all integers and ranges in between.

Compounds or compositions described herein can be formulated in any manner suitable for a desired delivery route, e.g., transtympanic injection, transtympanic wicks and catheters, cochlear implants, and injectable depots. In some instances, compositions or formulations include one or more physiologically-acceptable components, including derivatives or prodrugs, solvates, stereoisomers, racemates, or tautomers thereof with any physiologically acceptable carriers, diluents, and/or excipients.

As noted above, certain compositions are adapted for, and certain methods employ, administration to the middle ear or inner ear, for example, by local administration to the round window membrane. The membrane of the round window is the biological barrier to the inner ear space and represents the major obstacle for the local treatment of hearing impairment. The administered drug must overcome this membrane to reach the inner ear space. The drug can operatively (e.g., injection through the tympanic membrane) be placed locally to the round window membrane and can then penetrate through the round window membrane. Substances that penetrate the round window typically distribute in the perilymph and thus reach the hair cells and supporting cells.

The pharmaceutical formulations may also contain a membrane penetration enhancer, which supports the passage of the agents mentioned herein through the round window membrane. Accordingly, liquid, gel or foam formulations may be used. It is also possible to apply the active ingredient orally or to employ a combination of delivery approaches.

Certain compositions are adapted for, and certain methods employ, administration to the middle ear or inner ear, for example, by intratympanic or transtympanic administration. Intratympanic (IT) delivery of drugs to the ear is increasingly used for both clinical and research purposes. Some groups have applied drugs in a sustained manner using microcatheters and microwicks, while the majority have applied them as single or as repeated IT injections (up to 8 injections over periods of up to 2 weeks8).

Intratympanically applied drugs are thought to enter the fluids of the inner ear primarily by crossing the round window (RW) and oval window (OW) membranes. Calculations show that a major factor controlling both the amount of drug entering the ear and the distribution of drug throughout the ear is the duration the drug remains in the middle ear space. Single, ‘one-shot’ applications or applications of aqueous solutions for few hours' duration result in steep drug gradients for the applied substance. Since inner ear fluids are connected, a drug delivered to the inner ear will contact the vestibular organs and cochlea. The vestibular organs reside in close proximity to the oval window.

Other injection approaches include by osmotic pump, or, by combination with implanted biomaterial, and more preferably, by injection or infusion. Biomaterials that can aid in controlling release kinetics and distribution of drug include hydrogel materials, degradable materials. One class of materials that is most preferably used includes in situ gelling materials. All potential materials and methodologies mentioned in these references are included herein by reference. Other materials include collagen or other natural materials including fibrin, gelatin, and decellularized tissues. Gelfoam may also be suitable.

Delivery may also be enhanced via alternate means including but not limited to agents added to the delivered composition such as penetration enhancers, or could be through devices via ultrasound, electroporation, or high speed jet.

Methods described herein can also be used for inner ear cell types that may be produced using a variety of methods know to those skilled in the art including those cell types described in PCT Application No. W02012103012 A1.

With regard to human and veterinary treatment, the amount of a particular agent(s) that is administered may be dependent on a variety of factors, including the disorder being treated and the severity of the disorder; activity of the specific agent(s) employed; the age, body weight, general health, sex and diet of the patient; the time of administration, route of administration, and rate of excretion of the specific agent(s) employed; the duration of the treatment; drugs used in combination or coincidental with the specific agent(s) employed; the judgment of the prescribing physician or veterinarian; and like factors known in the medical and veterinary arts.

The agents described herein may be administered in a therapeutically effective amount to a subject in need of treatment. Administration of compositions described herein can be via any of suitable route of administration, particularly by intratympanically. Other routes include ingestion, or alternatively parenterally, for example intravenously, intra-arterially, intraperitoneally, intrathecally, intraventricularly, intraurethrally, intrasternally, intracranially, intramuscularly, intranasally, subcutaneously, sublingually, transdermally, or by inhalation or insufflations, or topical by ear instillation for absorption through the skin of the ear canal and membranes of the eardrum. Such administration may be as a single or multiple oral dose, defined number of ear drops, or a bolus injection, multiple injections, or as a short- or long-duration infusion. Implantable devices (e.g., implantable infusion pumps) may also be employed for the periodic parenteral delivery over time of equivalent or varying dosages of the particular formulation. For such parenteral administration, compositions are preferably formulated as a sterile solution in water or another suitable solvent or mixture of solvents. The solution may contain other substances such as salts, sugars (particularly glucose or mannitol), to make the solution isotonic with blood, buffering agents such as acetic, citric, and/or phosphoric acids and their sodium salts, and preservatives.

Compositions described herein can be administered by a number of methods sufficient to deliver the composition to the inner ear. Delivering a composition to the inner ear includes administering the composition to the middle ear, such that the composition may diffuse across the round window to the inner ear and administering a composition to the inner ear by direct injection through the round window membrane. Such methods include, but are not limited to auricular administration, by transtympanic wicks or catheters, or parenteral administration, for example, by intraauricular, transtympanic, or intravestibular injection.

In particular embodiments, the compositions and formulations of the disclosure are locally administered, meaning that they are not administered systemically.

In one embodiment, a syringe and needle apparatus is used to administer compounds or compositions to a subject using auricular administration. A suitably sized needle is used to pierce the tympanic membrane and a wick or catheter comprising the composition is inserted through the pierced tympanic membrane and into the middle ear of the subject. The device may be inserted such that it is in contact with the round window or immediately adjacent to the round window. Exemplary devices used for auricular administration include, but are not limited to, transtympanic wicks, transtympanic catheters, round window microcatheters (small catheters that deliver medicine to the round window), and Silverstein Microwicks™ (small tube with a “wick” through the tube to the round window, allowing regulation by subject or medical professional).

In some embodiments, a syringe and needle apparatus is used to administer compounds or compositions to a subject using transtympanic injection, injection behind the tympanic membrane into the middle and/or inner ear. The formulation may be administered directly onto the round window membrane via transtympanic injection or may be administered directly to the vestibula via intravestibular injection or directly to the vestibular organs via intravestibular injection.

In some embodiments, the delivery device is an apparatus designed for administration of compounds or compositions to the middle and/or inner ear. By way of example only: GYRUS Medical GmbH offers micro-otoscopes for visualization of and drug delivery to the round window niche; Arenberg has described a medical treatment device to deliver fluids to inner ear structures in U.S. Pat. Nos. 5,421,818; 5,474,529; and 5,476,446, each of which is incorporated by reference herein for such disclosure. U.S. patent application Ser. No. 08/874,208, which is incorporated herein by reference for such disclosure, describes a surgical method for implanting a fluid transfer conduit to deliver compositions to the inner ear. U.S. Patent Application Publication 2007/0167918, which is incorporated herein by reference for such disclosure, further describes a combined otic aspirator and medication dispenser for transtympanic fluid sampling and medicament application.

In some embodiments, a composition disclosed herein is administered to a subject in need thereof once. In some embodiments, a composition disclosed herein is administered to a subject in need thereof more than once. In some embodiments, a first administration of a composition disclosed herein is followed by a second, third, fourth, or fifth administration of a composition disclosed herein.

The number of times a composition is administered to an subject in need thereof depends on the discretion of a medical professional, the disorder, the severity of the disorder, and the subject's response to the formulation. In some embodiments, a composition disclosed herein is administered once to a subject in need thereof with a mild acute condition. In some embodiments, a composition disclosed herein is administered more than once to a subject in need thereof with a moderate or severe acute condition. In the case wherein the subject's condition does not improve, upon the doctor's discretion the composition may be administered chronically, that is, for an extended period of time, including throughout the duration of the subject's life in order to ameliorate or otherwise control or limit the symptoms of the subject's disease or condition.

In the case wherein the subject's status does improve, upon the doctor's discretion the composition may administered continuously; alternatively, the dose of drug being administered may be temporarily reduced or temporarily suspended for a certain length of time (i.e., a “drug holiday”). The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday may be from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once the subject's hearing and/or balance has improved, a maintenance dose can be administered, if necessary. Subsequently, the dosage or the frequency of administration, or both, is optionally reduced, as a function of the symptoms, to a level at which the improved disease, disorder or condition is retained. In certain embodiments, subjects require intermittent treatment on a long-term basis upon any recurrence of symptoms.

### EXAMPLES

### Example 1

Isolation of stem cells from the inner ear: All animal studies were conducted under an approved institutional protocol according to National Institutes of Health guidelines. For experiments with neonatal mice (postnatal days 1-3), the vestibular organs were dissected in HBSS. The vestibular organs were then treated with TrypLE (Life Technologies) for 15-20 minutes at 3T C. Single cells obtained by mechanical trituration were filtered (40 μm) and suspended in Matrigel (Corning) for 3D culture.

### Expansion of Sox2 and Sox9 Positive Cells

Cells were cultured in a 1:1 mixture of DMEM and F12, supplemented with Glutamax (GIBCO), N2, B27 (Invitrogen), EGF (50 ng/mL; Chemicon), bFGF (50 ng/mL; Chemicon), IGF1 (50 ng/mL; Chemicon) and a composition comprising the following agents: CHIR99021 (a Wnt activator), 616452 (a TGF-beta inhibitor), Noggin (a BMP4 inhibitor), VPA (HDAC inhibitor), and combinations there. Media were changed every other day.

Differentiation of Sox2 and Sox9 Progenitor Cells Stem cell colonies were differentiated in a 1:1 mixture of DMEM and F12, supplemented with Glutamax (GIBCO), N2, B27 (Invitrogen), with the addition of molecules that drive differentiation, or after removal of growth factors without the addition of molecules that drive differentiation. Small molecules were added to the culture to test their effect on differentiation.

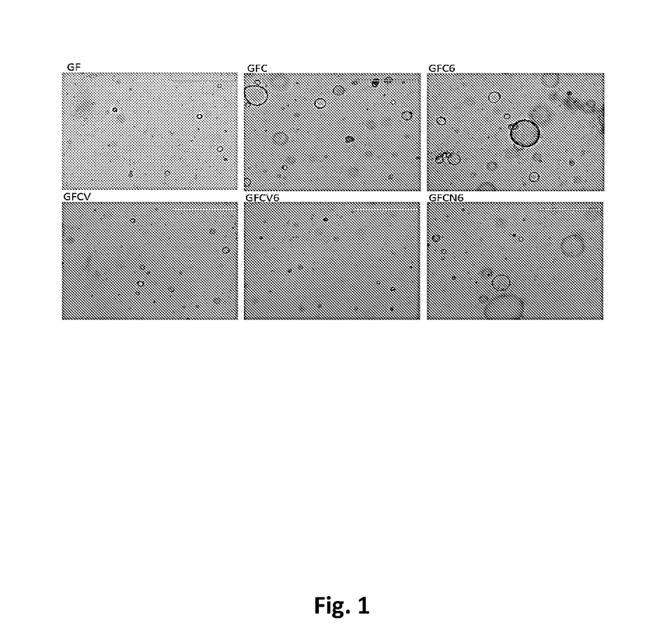

FIG. 1 shows transmitted light images of colonies in three-dimensional (3D) culture with various culture media conditions, as indicated.

### Analysis

Total cells were quantified after 10 days (D10) in culture in multiple conditions.

The treatment conditions were as follows:

(i) GF; (ii) GF+C (GFC); (iii) GF+C+6 (GFC6); (iv) GF+C+V (GFCV); (v) GF+C+V+6 (GFCV6); and (vi) GF+C+N+6 (GFCN6); wherein

a) GF=EGF, FGF, and IGF

b) C=CHIR99021

c) V=VPA

d) 6=616452

e) N=Noggin

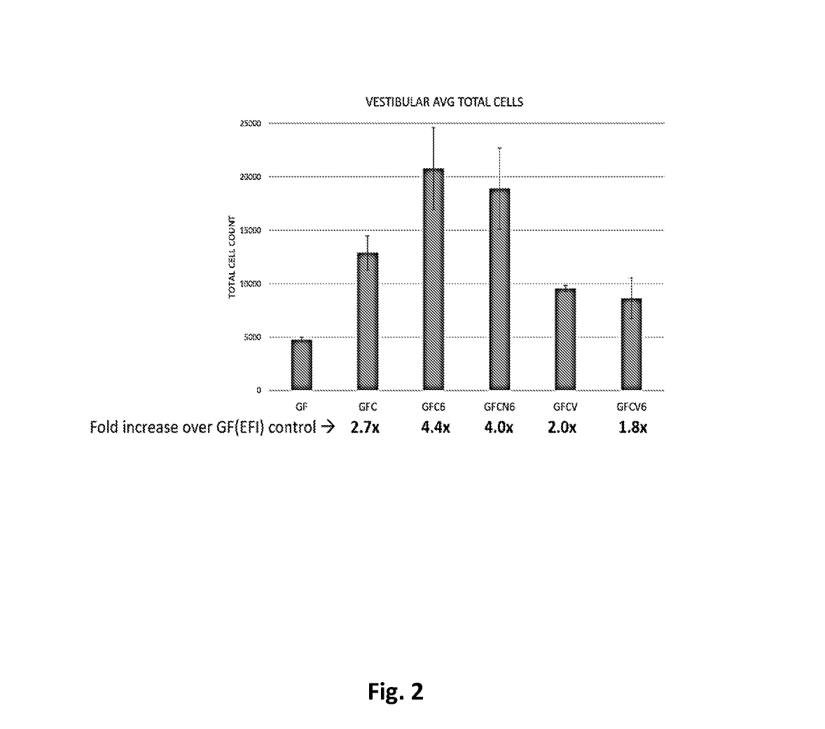

On D10, cell colonies were dissociated into single cells using TrypLE (Gibco). The cells were then stained with propidium iodide (PI) and analyzed using a flow cytometer expression. The number of cells were quantified. As shown in FIG. 2, Wnt agonism promoted supporting cell/stem cell growth, Wnt agonism+TGF-Beta inhibition improved stem cell growth, and HDAC inhibition inhibited growth.

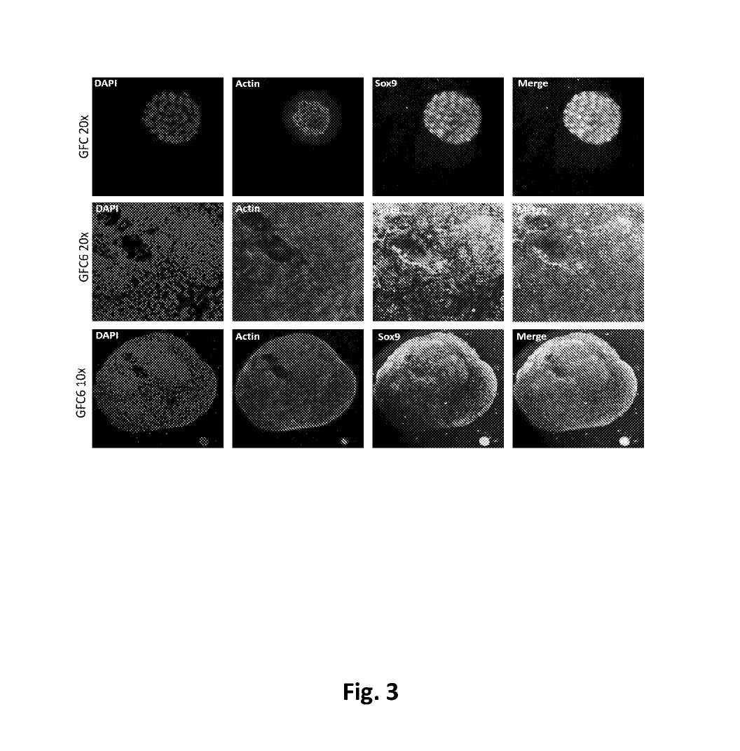

Also on D10, cells were stained with Sox2 (data not shown), as well as Sox9, Rhodamine Phalloidin (stains F-actin), and DAPI (nuclear stain) (see FIG. 3) to assess the identity of the cell populations. Colonies were visualized using confocal microscopy. Sox2 and Sox9 are stem cell markers in the balance organs.

FIG. 3 shows representative confocal images depicting clonal supporting cell/stem cell colonies with the characteristic actin lattices indicative of supporting cells (red), and the stem cell/supporting cell marker Sox9 (green).

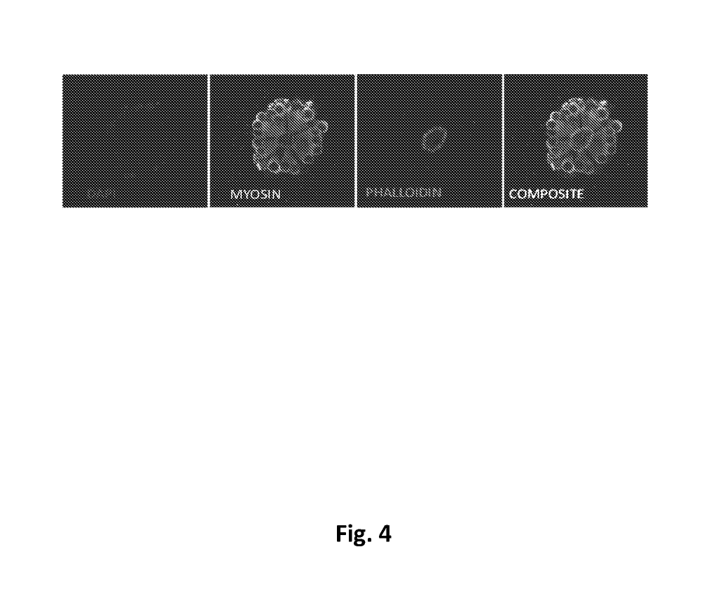

FIG. 4 show shows that progenitor colonies could be converted into high purity hair cell populations using gamma secretase inhibition with LY411575. Indicative of hair cells, the cells express Myo VIIa (green) and have actin hair bundles (red).

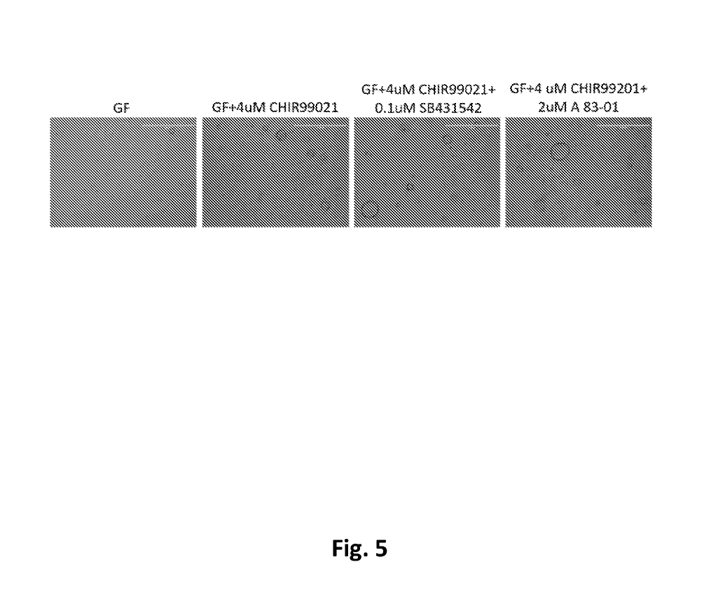

FIG. 5 shows that in a background of GF, (i) Wnt agonism (CHIR99021) promoted supporting cell/stem cell growth and colony formation, and (ii) Wnt agonism+TGF-beta inhibition using two alternative TGF-beta inhibitors (SB-431542 & A 83-01) generated larger supporting cell/stem cell colonies compared to Wnt agonism alone.

From the foregoing description, it will be apparent that variations and modifications may be made to the invention described herein to adopt it to various usages and conditions. Methods and materials are described herein for use in the present invention; other, suitable methods and materials known in the art can also be used. The materials, methods, and examples are illustrative only and not intended to be limiting. Such embodiments are also within the scope of the following claims. The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof. The teachings of all patents, published applications and references cited herein are incorporated by reference in their entirety. While this invention has been particularly shown and described with references to example embodiments thereof, it will be understood by those skilled in the art that various changes in form and details may be made therein without departing from the scope of the invention encompassed by the appended claims.

1. A method for expanding a population of vestibular cells in a vestibular tissue comprising contacting the vestibular tissue with (i) a Wnt agonist, a GSK3-alpha inhibitor, or a GSK3-beta inhibitor and (ii) a TGF-β Inhibitor to form an expanded population of cells in the vestibular tissue.

2. The method of claim 1 wherein the Wnt agonist, GSK3-alpha inhibitor, or GSK3-beta inhibitor and TGF-β inhibitor are capable in a stem cell proliferation assay of increasing the number of supporting cells in a stem cell proliferation assay cell population by a factor of at least 10 or at least 50.

3. The method of claim 2 wherein the Wnt agonist, GSK3-alpha inhibitor, or GSK3-beta inhibitor and/or TGF-β inhibitor are capable in a stem cell differentiation assay of forming hair cells from a cell population comprising vestibular supporting cells.

4. The method of claim 1 wherein the TGF-β Inhibitor is selected from 616452 (Repsox), Galunisertib (LY2157299), EW-719, IN-1130, EW-7203, EW-7195, Repsox, SM16, R 268712, GW788388, SB-431542, A-83-01, and PF-03671148.

5. The method of claim 1 wherein the vestibular tissue maintains Native Morphology.

6. The method of claim 1, wherein the vestibular tissue is in a subject.

7. The method of claim 6, wherein the contacting the vestibular tissue with the composition is achieved by administering the composition transtympanically to the subject.

8. The method of claim 6, wherein contacting the vestibular tissue with the composition results in improved vestibular functioning of the subject.

9. A method of facilitating the generation of tissue cells, the method comprising administering or causing to be administered to a stem cell population (i) a Wnt agonist, a GSK3-alpha inhibitor, or a GSK3-beta inhibitor and (ii) a TGF-β Inhibitor.

10. The method of claim 9 wherein the tissue cells are vestibular.

11. The method of claim 9 wherein the tissue cells are vestibular hair cells that are Type I and/or Type II hair cells.

12. A method of treating a subject who has, or is at risk of developing, a disease associated with absence or lack of certain tissue cells, the method comprising administering or causing to be administered to said subject (i) a Wnt agonist, a GSK3-alpha inhibitor, or a GSK3-beta inhibitor and (ii) a TGF-β Inhibitor.

13. The method of claim 12, wherein the tissue cells are vestibular cells.

14. The method of claim 13, wherein the tissue cells are vestibular hair cells comprise Type I vestibular hair cell and/or Type II vestibular hair cells.

15. A method of treating a subject who has, or is at risk of developing a vestibular condition, the method comprising administering to the subject (i) a Wnt agonist, a GSK3-alpha inhibitor, or a GSK3-beta inhibitor and (ii) a TGF-β Inhibitor.

16. The method of claim 15 wherein the compound is dispersed in a biocompatible matrix.

17. The method of claim 16 wherein the biocompatible matrix is a biocompatible gel or foam.

18. The method of claim 15, wherein the compound is administered transtympanically to a vestibular tissue of the subject.

19. The method of claim 1 wherein the Wnt agonist, GSK3-alpha inhibitor, or GSK3-beta inhibitor is selected from CHIR99021, LY2090314, AZD1080, and GSK3 inhibitor XXII.

20. The method of claim 1 wherein the TGF-β Inhibitor is selected from 616452 (Repsox), Galunisertib (LY2157299), EW-719, IN-1130, EW-7203, EW-7195, SM16, R 268712, GW788388, SB-431542, A-83-01, and PF-03671148.

21. The method of claim 1 further comprising an additional agent selected from a Notch activator, HDAC inhibitor, a BMP4 antagonist, upregulator of Sox2, Vitamin D (calcitriol), Vitamin B (nicotinomide), Vitamin A, Vitamin C (pVc), Lgr4, p38/MAPK inhibitor, ROCK inhibitor, TGF-beta RI kinase inhibitor, and/or an inhibitor of Alk2, Alk4, Alk5, and/or Alk7.

22. The method of claim 1, further comprising an epidermal growth factor (EGF), fibroblast growth factor (FGF), insulin-like growth factor (IGF), or a combination thereof.

23. A pharmaceutical composition, comprising a pharmaceutically-acceptable carrier and (i) a Wnt agonist, a GSK3-alpha inhibitor, or a GSK3-beta inhibitor and (ii) a TGF-β inhibitor, wherein the composition is adapted for administration to the middle ear and/or inner ear.

24. The pharmaceutical composition of claim 23, wherein (i) and (ii) are dispersed in a biocompatible matrix.

25. The pharmaceutical composition of claim 24, wherein the biocompatible matrix is a biocompatible gel or foam.

26. The pharmaceutical composition of claim 23, wherein the composition is adapted for local administration to the round window membrane

27. The pharmaceutical composition of claim 23, wherein the composition is adapted for transtympanic administration, optionally to vestibular tissue.

28. The pharmaceutical composition of claim 23, wherein the Wnt agonist, GSK3-alpha inhibitor, or GSK3-beta inhibitor is selected from CHIR99021, LY2090314, AZD1080, and GSK3 inhibitor XXII.

29. The pharmaceutical composition of claim 23, wherein the TGF-β inhibitor is selected from 616452 (Repsox), Galunisertib (LY2157299), EW-719, IN-1130, EW-7203, EW-7195, SM16, R 268712, GW788388, SB-431542, A-83-01, and PF-03671148.

30. The pharmaceutical composition of claim 23, further comprising an additional agent selected from a Notch activator, HDAC inhibitor, a BMP4 antagonist, upregulator of Sox2, Vitamin D (calcitriol), Vitamin B (nicotinomide), Vitamin A, Vitamin C (pVc), Lgr4, p38/MAPK inhibitor, ROCK inhibitor, TGF-beta RI kinase inhibitor, and/or an inhibitor of Alk2, Alk4, Alk5, and/or Alk7.

31. The pharmaceutical composition of claim 23, further comprising an epidermal growth factor (EGF), fibroblast growth factor (FGF), insulin-like growth factor (IGF), or a combination thereof.

32. The pharmaceutical composition of claim 23, comprising a poloxamer.

33. The pharmaceutical composition of claim 31, wherein the poloxamer comprises at least one of Poloxamer 188 and Poloxamer 407 or mixtures thereof.

34. The pharmaceutical composition of claim 31, wherein the poloxamer is in a concentration between about 5 wt % and about 25 wt % relative to the composition.

35. The pharmaceutical composition of claim 33, wherein the poloxamer is in a concentration between about 10 wt % and about 23 wt % relative to the composition.

36. The pharmaceutical composition of claim 34, wherein the poloxamer is in a concentration between about 15 wt % and about 20 wt % relative to the composition.

37. The pharmaceutical composition of claim 35, wherein the poloxamer is in a concentration is approximately 17 wt % relative to the composition.

38. The pharmaceutical composition of claim 23, wherein the Wnt agonist, GSK3-alpha inhibitor, or GSK3-beta inhibitor is at a concentration of about 0.01 uM to 1000 mM, about 0.1 uM to 1000 mM, about 1 uM to 100 mM, about 10 uM to 10 mM, about 1 uM to 10 uM, about 10 uM to 100 uM, about 100 uM to 1000 uM, about 1 mM to 10 mM, or about 10 mM to 100 mM; or at a concentration ratio of about 0.01 to 1,000,000 fold relative to its Effective Stemness Driver Concentration, or about 0.1 to 100,000 fold relative to its Effective Stemness Driver Concentration, or about 1 to 10,000 fold relative to its Effective Stemness Driver Concentration, or about 100 to 5000 fold relative to Effective Stemness Driver Concentration, or about 50 to 2000 fold relative to its Effective Stemness Driver Concentration, or about 100 to 1000 fold relative to its Effective Stemness Driver Concentration, or at about 1000 fold relative to its Effective Stemness Driver Concentration; or at a concentration of about 0.01 nM to 1000 uM, about 0.1 nM to 1000 uM, about 1 nM to 100 uM, about 10 nM to 10 uM, about 1 nM to 10 nM, about 10 nM to 100 nM, about 100 nM to 1000 nM, about 1 uM to 10 uM, or about 10 uM to 100 uM, optionally wherein the Effective Stemness Driver Concentration is measured in an Lgr5 proliferation assay.

39. The pharmaceutical composition of claim 23, wherein the Wnt agonist, GSK3-alpha inhibitor, or GSK3-beta inhibitor is CHIR99021, which is at a concentration of about 1 uM to 1000 mM, about 10 uM to 100 mM, about 100 uM to 100 mM, about 1 mM to 10 mM, or about 1, 2, 3, 4, 5, 6, 7, 8, 9, or 10 mM; or at a concentration of about 1 nM to 1000 uM, about 10 nM to 100 uM, about 100 nM to 100 uM, about 1 uM to 10 uM, or about 1, 2, 3, 4, 5, 6, 7, 8, 9, or 10 uM.

40. The pharmaceutical composition of claim 23, wherein the Wnt agonist, GSK3-alpha inhibitor, or GSK3-beta inhibitor is LY2090314, which is at a concentration of about 0.01 uM to 1000 mM, about 0.1 uM to 10 mM, about 1 uM to 1 mM, about 10 uM, about 20 uM, about 30 uM, about 40 uM, or about 50 uM; or at a concentration of about 0.01 nM to 1000 uM, about 0.1 nM to 10 uM, about 1 nM to 1 uM, about 1 nM to 100 nM, or about 10 nM.

41. The pharmaceutical composition of claim 23, wherein the Wnt agonist, GSK3-alpha inhibitor, or GSK3-beta inhibitor is AZD1080, which is at a concentration of about 0.1 uM to 1000 mM, about 1 uM to 1000 mM, about 10 uM to 100 mM, about 100 uM to 10 mM, about 1 mM to 10 mM, or about 1, 2, 3, 4, 5, 6, 7, 8, 9, or 10 mM; or at a concentration of about 1 nM to 1000 uM, about 10 nM to 1000 uM, about 100 nM to 100 uM, about 1 uM to 10 uM, or about 1, 2, 3, 4, 5, 6, 7, 9, or 10 uM.

42. The pharmaceutical composition of claim 23, wherein the Wnt agonist, GSK3-alpha inhibitor, or GSK3-beta inhibitor is GSK3 inhibitor XXII, which is at a concentration of about 0.1 uM to 1000 mM, about 1 uM to 100 mM, about 10 uM to 10 mM, about 100 uM to 10 mM, about 100 uM to 1 mM, or about 1, 2, 3, 4, 5, 6, 7, 8, 9, or 10 mM; or at a concentration of about 0.1 nM to 1000 uM, about 1 nM to 100 uM, about 10 nM to 10 uM, about 100 nM to 1 uM, or about 0.5 uM.

43. The pharmaceutical composition of claim 23, wherein the TGF-beta inhibitor is at a concentration of about 0.01 uM to 1000 mM, about 0.1 uM to 1000 mM, about 1 uM to 100 mM, about 0.1 uM to 1 uM, about 1 uM to 10 uM, about 10 uM to 100 uM, about 100 uM to 1 mM, about 1 mM to 10 mM, or about 100 mM to 1000 mM, or about 10 mM to 100 mM, or about 100 mM to 1000 mM; or at a concentration ratio of about 0.1 to 1,000,000 fold relative to its Effective TGF-beta Concentration, or about 1 to 100,000 fold relative to its Effective TGF-beta Concentration, or about 10 to 10,000 fold relative to its Effective TGF-beta Concentration, or about 100 to 1000 fold relative to its Effective TGF-beta Concentration, or about 1000 fold relative to its Effective TGF-beta Concentration; or at a concentration of about 0.01 nM to 1000 uM, or about0.1 nM to 1000 uM, about 1 nM to 100 uM, about 10 nM to 10 uM, about 1 nM to 10 nM, about 10 nM to 100 nM, about 100 nM to 1000 nM, about 1 uM to 10 uM, about 10 uM to 100 uM, or about 100 uM to 1000 uM, optionally wherein the Effective TGF-beta Concentration is measured in an Lgr5 proliferation assay.

44. The pharmaceutical composition of claim 23, wherein the TGF-beta inhibitor is 616452 (Repsox) at a concentration of about 1 uM to 1000 mM, or about 10 uM to 1000 mM, or about 100 uM to 10 mM, or about 2 mM; or at a concentration of about 1 nM to 1000 uM, about 10 nM to 100 uM, about 100 nM to 10 uM, or about 2 uM.

45. The pharmaceutical composition of claim 30, wherein the BMP4 antagonist is at a concentration of about 0.01 uM to 1000 mM, 0.1 uM to 1000 mM, about 1 uM to 100 mM, about 10 uM to 10 mM, about 0.1 uM to 1 uM, about 1 uM to 10 uM, about 10 uM to 100 uM, about 100 uM to 1 mM, about 1 mM to 10 mM, about 10 mM to 100 mM, about 100 mM to 1000 mM; or at a concentration ratio of about 0.1 to 1,000,000 fold relative to its Effective BMP4 Antagonist Concentration, or about 1 to 100,000 fold relative to its Effective BMP4 Antagonist Concentration, or about 10 to 10,000 fold relative to its Effective BMP4 Antagonist Concentration, or about 100 to 1000 fold relative to its Effective BMP4 Antagonist Concentration, or about 1000 fold relative to its Effective BMP4 Antagonist Concentration; or at a concentration of about 0.01 nM to 100 uM, about 1 nM to 100 uM, about 10 nM to 10 uM, about 1 nM to 10 nM, about 10 nM to 100 nM, about 100 nM to 1000 nM, about 1 uM to 10 uM, about 10 uM to 100 uM, or about 100 uM to 1000 uM, optionally wherein the Effective BMP4 Antagonist Concentration is measured in an Lgr5 proliferation assay.

46. The pharmaceutical composition of claim 30, wherein the BMP4 antagonist is DMH1 at a concentration of about 1 uM to 1000 mM, about 10 uM to 100 mM, about 100 uM to 10 mM, or about 1 mM; or at a concentration of about 1 nM to 1000 uM, or about 10 nM to 100 uM, about 100 nM to 10 uM, or about 1 uM.

47. The pharmaceutical composition of claim 30, wherein the BMP4 antagonist is Noggin at a concentration of about 1 ug/ml to 10,000 ug/ml, about 10 ug/ml to 1000 ug/ml, or about 100 ug/ml; or at a concentration of about 1 ng/ml to 10,000 ng/ml, about 10 ng/ml to 1000 ng/ml, or about 100 ng/ml.

48. The pharmaceutical composition of claim 30, wherein the HDAC inhibitor is at a concentration of about 0.01 uM to 100,000 mM, about 1 uM to 10,000 mM, about 10 uM to 10,000 mM, about 100 uM to 1000 mM, about 1 uM to 10 uM, about 10 uM to 100 uM, about 100 uM to 1000 uM, about 1000 uM to 10 mM, about 10 mM to 100 mM, about 100 mM to 1000 mM, or about 1000 mM to 10,000 mM; or at a concentration ratio of about 0.1 to 1,000,000 fold relative to its Effective Concentration, or about 1 to 100,000 fold relative to its Effective Concentration, or about 10 to 10,000 fold relative to its Effective Concentration, or about 100 to 1000 fold relative to its Effective Concentration, or about 1000 fold relative to its Effective Concentration; or at a concentration of about 0.01 nM to 100,000 uM, about 1 nM to 10,000 uM, about 10 nM to 10,000 uM, about 100 nM to 1000 uM, about 1 nM to 10 nM, about 10 nM to 100 nM, about 100 nM to 1000 nM, about 1 uM to 10 uM, about 10 uM to 100 uM, about 100 uM to 1000 uM, or about 1000 uM to 10,000 uM, optionally wherein the Effective Concentration is measured in an Lgr5 proliferation assay.

49. The pharmaceutical composition of claim 30, wherein the HDAC inhibitor is valproic acid at a concentration of about 10 uM to 100,000 mM, about 1 mM to 10,000 mM, about 10 mM to 10,000 mM, about 100 mM to 10,000 mM, about 200 mM to 2000 mM, about 1000 mM, or about 600 mM; or at a concentration of about 10 nM to 100,000 uM, 1 uM to 10,000 uM, about 10 uM to 10,000 uM, about 100 uM to 10,000 uM, about 200 uM to 2000 uM, or about 1000 uM.

50. The pharmaceutical composition of claim 23, for use in expanding a population of vestibular cells in a vestibular tissue.

51. The pharmaceutical composition of claim 23, for use in treating a subject how has, or is at risk of developing, a disease associated with absence or lack of vestibular cells, optionally Type I vestibular hair cell and/or Type II vestibular hair cells.

52. The pharmaceutical composition of claim 23, for use in treating a subject who has, or is at risk of developing, a vestibular condition.


SEQUENCE LISTING (1 sequence)

SEQ ID NO: 1 - protein, 17 aa, Homo sapiens
CDDYYYGFGCNKFCRPR